SEQUENCE LISTING (30 sequences)

SEQ ID NO: 1 - protein, 409 aa, Rhodosporidium toruloides
MATHTFASPPTRFTVDIPQSELDELDFRLDKTRWPATEIVPEDGADDPTAFGLGAGPTLPLMKELAKGWREFDWKKAQDHLNTFEHYTVEIEDLSIHFLHHRSTRPNAVPLILCHGWPGHFGEFLNVIPLLTEPSDPSAQAFHVVAPSMPGYAWSSPPPSSKWSMPDTARVFDKLMTGLGYEKYMAQGGDWGSIAARCLGSLHKDHCKAVHLNFLPVFPPVPMWLINPHTLLAWAPRFLVPEKQAARMKRGLAYLEKGSAYYVMQQLTPRTPAYGLTDSPVGLLAWIGEKFEPTIQEASKQAQPTLTRDELYFTCSLYWFTRSIGTSFLPYSLNPHFTTFLTDSRYHLPNFALSLYPGEIYCPAERDAKRTGNLKWIKDAPDGGHFAALEKPDVFVEHLREAFGVMWEK

SEQ ID NO: 2 - protein, 409 aa, Rhodosporidium toruloides
MATHTFASPPTRFTVDIPQSELDELHSRLDKTRWPATEIVPEDGTDDPTAFGLGAGPTLPLMKELAKGWREFDWKKAQDHLNTFEHYMVEIEDLSIHFLHHRSTRPNAVPLILCHGWPGHFGEFLNVIPLLTEPSDPSAQAFHVVAPSMPGYAWSLPPPSSKWNMPDTARVFDKLMTGLGYEKYMAQGGDWGSIAARCLGSLHKDHCKAVHLNFLPVFPPVPMWLINPHTLLAWAPRFLVPEKQAARMKRGLAYLEKGSAYYVMQQLTPRTPAYGLTDSPVGLLAWIGEKFEPTIQEASKQAQPTLTRDELYFTCSLYWFTRSIGTSFLPYSLNPHFTTFLTDSKYHLPNFALSLYPGEIYCPAERDAKRTGNLKWIKDAPEGGHFAALEKPDVFVEHLREAFGVMWEK

SEQ ID NO: 3 - protein, 411 aa, Rhodosporidium paludigenum
MAAHSFTAPPAPYNIDFAPQVNDLHRRLDAARWPEHDVVPDDVDHGEHGAFGLGAGPSLALMKELAQEWRGQDQKQLQDHLNSYKNYRVEIEGLNIHFLHYPSSRADAFPLILCHGWPGGYHEFLHVLERLTEPEDQGSRAFHVVVPSMPGYAFSSPPKTAKWGMEDTARVFDKLMTGLGYAKYAAQGGDWGSITARCLGSLHKENCVAVHLNFCPVPPPFPLNMFNPRTLLDWMPRFVLPDQRRAKIERGVAYIERGSAYYAMQNLTPRTPAYGLNDSPIGLLAWIGEKMIPGIDKAVKHPNATLNREALFTTLSIYWFTGSIGSSFLPYALNPHFSTFLVSPRHQLPNFALSNFPDELFTPEERDARRTGNLRWYKDAEDGGHFAALEKPEVFAEHVREAMGVLLSNQA

SEQ ID NO: 4 - protein, 410 aa, Rhodotorula araucariae
MSEHSFEAPPQPFTVDFAPHIEDLHRRLDNARWPTQEIVPVDVSETEHHNAFGLGMGPQLNLMKELANGWRAFDQSALQDHLNSFNNWKVEIEGLSIHFLHHRSTRAGALPLILCHGWPGGYHEFLHVVQLLTEPEGADAQAFHLVVPSMPGYAFSSPPPTAKWGMEDTARVFDKLMTGLGYNKYVAQGGDWGSITARCLGALHKDHCIAVHLNFCPVPPPFPFDQFNPRTLLNWMPRFVISDQQRAKLERGLAYLEKGSAYYVMQQLTPRTPAYALNDSPIGLLAWIGEKMIPGINEASAQPNPTLNRDALYTTLSLYWFTNSIGTSFLPYSLNPHFSTFLTSPRYRLPNFALSSFPGELFSPTPRDAARTGVMRWYKEADDGGHFAALEKPDVFSQHVREAVKAMLSS

SEQ ID NO: 5 - protein, 409 aa, Cryptococcus curvatus
MATHTFASPPTRFTVDIPQSEVDQLHSRLDKTRWPGTEIVPEDGADDPTAFGLGAGPTLPLMKELAKGWRDFDWKKAQDHLNTFEHYTVEIEDLSIHFLHHRSTRPNAVPLILCHGWPGHFGEFLHVIPLLTEPSDPSAQAFHVVAPSMPGYAWSSPPPTSKWNMPDTARVSDKLMNGLGYEKYMAQGGDWGSIAARCLGALHKDHCKAVHLNFLPVFPPVPMWLINPHTLLAWAPRFLVPEKQAARMKRGLAYLEKGSAYYVMQQLTPRTSAYGLTDSPVGLLAWIGEKFEPTIQEASKQAQPTLTRDELYFTCSLYWFTRSIGTSFLPYSLNPHFTTFLTDSKYYLPNFALSLYPGEIYCPAERDAKRTGNLKWIKDAPEGGHFAALEKPDVFVDHLREAFGVMWEK

SEQ ID NO: 6 - protein, 401 aa, Filobasidiella (Cryptococcus) neoformans var neofs
MSYSDLPHKPTIPVEPFKLSVPHEDLNGLLTLLKSTRIAKESYENVSAHENKFGITRKWLVNMKDEWIKQDWRKQEERINSLPAFKAKVKNSDGSVFSIHFTALFSKKKVAIPIILSHGWPGSFYEFVPMMEMVKKKYSPEDLPFHLIVPSLPGWLFSTPPPNDREFNVTDVGYLFNGLMEGLGFGDGYVAQGGDIGSYVTNELGAKYPACKIIHVNYSNPPPRPLPSPGSPGQEASPPSAEDLLELLQKFGYALEHSTRPATVGLVVGSNPLSLLAWVGEKFLEWTDESPSEETILTMTSLYWFTDCFTTSIYTYRYGLGAKRHESAKQASYQKCPLGYSQFPKEIVEIPAEWVKAQSNMVWSKKHESGGHFAALEKPELLWADIEEFVHSQWENYKGNY

SEQ ID NO: 7 - protein, 394 aa, Rhodotorula mucilaginosa
MPARSLTLRPFSPSFTAPELDGLARSLESSRLPAETYASRQAKYGIKHAWMKNALQRWKDGFDWKKHEQDINEVDHYMVQVQSDGIQHDLHVIYHESKDPNAIPLLLLHGWPGSAFEFIEAIKILRKSTSPAFHLIAPMEPGYGWSTPPPLDRGFNMNDCTALMNDLMVGLGYGDGYAAQGGDIGSGLARLLAVNYDACKCININYMPAVAPPEDAPERHQIKPHEEDALRRADEFQKTGRGYANMHATRPGTVGIVVGSSPVALLAWIAEKYLAWTDEDPPLDTILAICTIWWIRDSYPSSIWAYADFLETGISALHNDPKYKLDKKPFGFSSFKEEISATPEAWAGRNGNLQFYRYHDKGGHFAALEQPEAFAQDMQDCFGKIWPLSQEQKS

SEQ ID NO: 8 - DNA, 1230 bp, Rhodosporidium toruloides
atggcgacacacacattcgcttcgcctcccacgcgcttcaccgtcgacatcccacagtcagaactcgacgaactcgacttccgactcgacaagacccgctggccggcgacagagatcgttccagaggatggggcggacgacccgacggcgtttgggctcggagcagggccgacgctgccgctcatgaaggaactggcgaagggttggcgcgagttcgactggaagaaggcgcaggaccacctcaacacctttgagcactacacggtcgaaatcgaggacctctccatccacttcctccaccaccgctcgactcgcccgaatgctgttccgctcatcctctgccacggctggccaggccacttcggcgagttcctgaacgtcataccgctcttgacggagccgtcggacccgtccgcccaggcgttccacgtcgtcgcgccttcgatgcccggttatgcttggtcttcgcctcctccgtcctccaagtggagcatgcctgataccgcgagggtcttcgacaagctcatgaccgggcttggctacgagaagtacatggcgcagggcggagactggggcagcatcgctgctcgctgccttggatcgcttcacaaagaccactgcaaagccgtccacctcaacttcctccccgtcttcccacccgtcccgatgtggcttatcaacccgcacacgctccttgcctgggcaccgcgcttcctcgtgccggagaagcaggctgcgcgtatgaagcgcgggttggcgtaccttgagaagggctccgcctactacgtcatgcagcagttgacgcctcgcacgcctgcgtacggcctgaccgacagtcccgtcggcttgctggcctggatcggcgagaagttcgagccgaccattcaggaggcgagcaagcaagcccagccgaccctgactcgcgacgagctctacttcacctgctcgctctactggttcacccgctcaatcggcacctccttccttccctactcgctcaacccgcacttcaccaccttcctgaccgacagcaggtaccacctgcccaactttgccctgtccctctacccgggcgagatctactgcccggcagaacgggacgccaagcgtaccggcaacctcaagtggatcaaggacgcgcccgatggaggacactttgctgcgctcgagaagcccgatgtgtttgtcgagcatctcagggaggcgtttggcgtcatgtgggagaagtag

SEQ ID NO: 9 - DNA, 1230 bp, Rhodosporidium toruloides
atggcgacacacacattcgcttcgcctcccacccgcttcaccgtcgacatcccacagtcggaactcgacgaacttcactcgcgactcgacaagacccgctggccggcgacagagatcgttccagaggatgggacggacgatccgacggcgtttgggctcggagcagggccgacgctgccgctcatgaaggaattggcgaagggttggcgcgagttcgactggaaaaaggcgcaggaccacctcaacaccttcgagcactacatggtcgaaattgaggacctctcgatccacttcctccaccatcgctcgactcgcccgaacgctgttcccctcatcctctgccacggctggccaggccactttggcgagttcctgaacgttatcccgctcttgacggagccgtcggacccctccgctcaggcgttccacgtcgtcgccccttcgatgcctggctatgcttggtctttgcctcctccgtcctccaagtggaacatgcctgacaccgcgagggtcttcgacaagctcatgaccgggcttggctacgagaagtacatggcgcagggcggagactggggaagcatcgccgctcgctgccttggatcgctgcacaaggaccattgcaaagccgtccacctcaacttcctccccgtcttcccacccgtcccgatgtggcttatcaacccgcacacgctccttgcctgggcaccgcgcttcctcgtgccggagaagcaggctgcgcgtatgaagcgcgggttggcgtaccttgagaagggctccgcctactacgtcatgcagcagttgacgcctcgcacgcctgcgtacggcctgaccgacagtcccgtcggcttgctggcctggatcggcgagaagttcgagccgaccattcaggaggcgagcaagcaagcccagccgaccctgactcgcgacgagctctacttcacctgctcgctctactggttcacccgctcaatcggcacctccttccttccctactcgctcaacccgcacttcaccaccttcctgaccgacagcaagtaccacctgcccaactttgccctctcgctttacccaggcgagatctactgccccgccgagcgggacgccaagcgcaccggcaacctcaagtggatcaaggacgcgcctgagggaggacactttgctgcgctcgaaaagccggatgtgtttgtcgagcacctcagggaggcgtttggcgtcatgtgggagaagtag

SEQ ID NO: 10 - DNA, 1236 bp, Rhodosporidium paludigenum
atggctgcccattcctttactgcacctcctgcaccctacaacatcgactttgcgccccaggtaaatgacctgcaccgccgtctcgatgctgcccgctggccggaacacgacgtggtgcccgacgatgtggatcacggagagcacggcgcattcggactcggcgctggtcccagcctcgccctcatgaaggagctcgcgcaggaatggaggggccaggaccagaagcagctgcaggaccacctcaactcctacaagaactatcgcgtcgagatcgagggtctcaacatccacttcctgcactacccgtcgtctcgcgccgatgcgttcccgctcatcctgtgccacggctggcctggcggctaccacgagttcctgcacgtcctagagcgcctcacggagcccgaggatcaggggtcgcgggccttccatgtcgtcgtgccttccatgccgggttacgccttctcctcgccgcccaagacggcaaaatggggcatggaggacacggctcgcgtgttcgacaagctcatgacggggctaggttacgccaagtatgcggcccaaggcggtgactgggggtctatcacggcgcgctgcctaggttcgctgcacaaggagaactgcgtcgctgtccacctcaacttctgcccggttcccccgccgttcccgctcaacatgttcaacccgcgcacacttctggactggatgcctcgctttgtcctgcctgatcaacggcgggccaagattgagcgcggcgtggcctatatcgagcgcggctctgcctactacgccatgcaaaacttgacgccgcgcacgcctgcgtacggcttgaacgatagtccgattggtttgctcgcgtggattggcgagaagatgattccgggcattgacaaggctgtcaagcatccgaacgcaaccctcaatcgcgaagctcttttcacgacactctcgatctactggttcacgggctcgattggctcctccttcctgccatacgctctcaacccgcacttctctaccttcctcgtctcgccgcggcaccaactgccgaactttgctctgtccaactttcccgacgagctgttcacgcccgaagaacgcgatgctcgccgaaccggaaacttgcggtggtacaaggatgcagaggatggagggcacttcgcggcgctggagaagcccgaggtcttcgccgagcacgtaagggaggcgatgggggtcttgctgtcgaaccaggcctga

SEQ ID NO: 11 - DNA, 1233 bp, Rhodotorula araucariae
atgagcgagcacagcttcgaggccccgccacagccgtttacggtcgactttgctccccacatcgaggatctccaccgccgtctcgacaatgcgcgctggccgacgcaagagattgtccccgtcgacgtgtccgagacggagcatcacaacgcgttcggactcgggatgggcccgcagctcaaccttatgaaggagctcgccaacggctggcgcgcgttcgaccagtcggcgctccaggaccacctcaacagcttcaacaactggaaggtcgagatcgagggattgtcgatccacttcctccaccatcgctcgacgcgcgccggcgctctcccgctcatcctgtgccatggctggcccggcgggtaccacgagttcctccacgtcgtccagctcctcaccgaaccagagggggcggatgcgcaggcgtttcacctcgtcgtcccctcgatgcccgggtacgccttctcgtctccgccgccgacggccaagtggggcatggaagacactgcaagggtttttgacaagctcatgaccggtttggggtacaacaagtatgtcgcgcagggcggtgactgggggtccatcacggcgcgatgcctcggcgcgctgcacaaggaccactgcattgctgtccacctcaacttctgccccgtcccgccgccgttcccattcgaccagttcaacccgcgcacgctgctcaactggatgccgcgcttcgtgatctcggaccagcagcgtgcgaagctcgagcgtgggctggcgtacctcgagaaggggtctgcttactatgtcatgcagcagctcacaccgcgtaccccggcctacgctctcaatgacagcccgattggcctgctcgcctggattggcgaaaagatgatcccaggcatcaacgaggcgagcgcgcagccgaacccgacgctcaatcgcgatgcgttgtacaccacgctctcgctgtactggttcaccaactccatcggcacctctttcctcccctactcgcttaacccgcacttcagcacgttcctcacctcgccccgctatcgcctgccgaactttgcgctgtcttccttcccgggcgagctgttctcgccgacgccgcgcgatgctgcgaggacgggcgtgatgcgctggtacaaggaggcggacgatggcgggcactttgcggcgctcgagaagcccgatgtgttcagccagcatgtcagggaggcagtcaaggccatgctgtcgtcgtga

SEQ ID NO: 12 - DNA, 1230 bp, Cryptococcus curvatus
atggcgacacacacattcgcttcgcctcccacccgcttcaccgtcgacattccgcagtcggaagttgaccaacttcactcgcgactcgataagactcgctggccagggacagagatcgttccagaggatggggcggacgacccgacggcgtttggactcggagcaggaccaacgctgccgctcatgaaggaactggcgaagggttggcgcgacttcgactggaagaaggcgcaggaccacctcaacaccttcgagcactacacggtggagatcgaggacctctcgatccacttcctccaccatcgctcgactcgtccgaatgctgttcccctcatcctctgccacggctggccaggacacttcggcgagtttctacacgttataccactcttgacggagccgtcggacccgtccgctcaggcgtttcacgtcgtcgcgccttcgatgcctggttatgcttggtcttcgcctcctcctacctccaagtggaatatgcctgacaccgcaagggtgtccgacaagcttatgaacggactcggctacgagaagtacatggcgcagggcggagactggggcagcatcgccgctcgctgcctcggagcgctgcacaaagatcactgcaaagccgtccacctcaacttcctccccgtcttcccgcccgtccctatgtggctcatcaacccacacacactcctcgcatgggctccgcgcttccttgtgccggagaagcaggctgcgcgcatgaagcgcgggttggcgtacctcgaaaagggctccgcctactacgtcatgcagcaattgacgcctcgcacgtctgcgtacggccttactgacagtcccgtcggcttgctggcctggatcggcgagaagtttgagccgaccattcaggaagcgagcaagcaagcccagccgaccctcactcgcgacgagctctacttcacctgctccttgtactggttcacccgctcaatcggcacctccttcctgccctactcgctcaacccgcacttcacgaccttcctgaccgacagcaagtattacctgcccaattttgccctctccctgtacccgggcgagatctactgccctgccgagcgggacgccaagcgcactggcaacctcaagtggatcaaggacgcgcctgagggaggacactttgcggcgctcgagaagcccgacgtgtttgtcgaccatctcagggaggcatttggcgttatgtgggagaagtag

SEQ ID NO: 13 - DNA, 1206 bp, Filobasidiella  neoformans var neoformans
atgtcgtattcagaccttccccacaagccgaccattcctgtcgagcccttcaaactatccgtaccccatgaggacctcaatgggctcctcactctactcaaatctaccagaattgccaaagaaagctatgagaatgtctctgcgcacgagaacaaatttggaataacgaggaaatggctggtgaatatgaaagatgaatggatcaaacaggactggcggaaacaagaagagcgaatcaattctcttcctgcgttcaaggcaaaggtaaaaaactcagatggttcggtattttcaatccattttaccgcccttttctcgaaaaagaaggtcgcaatccctatcatcctcagtcacggctggccaggtagcttttacgaattcgttccaatgatggagatggtgaagaagaaatatagtcctgaggatcttccgttccatttaatcgtaccatcccttcccggatggctcttctctacacctcctcctaatgatcgagaattcaatgtgaccgatgttgggtatctcttcaatggtctaatggagggcctaggtttcggagacggctatgttgcccaaggcggtgatattgggagctacgtgacgaacgaacttggtgccaagtacccagcttgcaaaatcattcacgtcaattactctaaccctcctcctcgcccacttccctctccaggctctcctggacaagaggcttcaccgccctctgctgaagatcttcttgagctactgcagaagtttggatatgctctcgagcattctactcgaccagccactgtaggcttggtagtaggctccaatccactcagcttactagcatgggtcggcgagaaattcctcgagtggactgatgagtccccaagtgaggaaacaattttgacaatgacaagcctttattggttcactgattgtttcacgacgtcgatttacacatacagatatggccttggtgctaaacgccatgaaagtgccaaacaagcttcttaccagaaatgtcctctgggctacagccagttccccaaagagatcgtcgagatacctgccgagtgggtcaaggcgcaatccaatatggtttggtctaagaaacatgaatccgggggtcattttgctgctctcgaaaagccagaattgttgtgggcggatatagaggagtttgtacactcgcagtgggagaattacaaaggtaattactga

SEQ ID NO: 14 - DNA, 1185 bp, Rhodotorula mucilaginosa
atgcccgcccgctcgctcacgctgcgcccgttctcgccgtcgttcacggctccggaactggacggtctcgctcgctcgctcgagtcgtcgcgcttgcccgccgagacgtacgcttcccgccaggccaaatacggcatcaagcatgcttggatgaagaatgccctccaacggtggaaggacgggttcgattggaagaagcacgagcaggacatcaacgaggtcgaccactatatggtgcaggtccagtccgatggcattcaacacgacctccatgtgatctatcacgaatcgaaagacccgaatgcgatcccgctcttgctgctgcatgggtggcccggttccgcgttcgagtttatcgaggcgatcaagatccttcgcaagagtacctcgcccgcgttccacctgatcgcgcccatggagcccggctacgggtggagtactccgccgccactcgaccgcggtttcaacatgaacgattgcacagcgctcatgaacgacttgatggttggactcgggtacggagacggttacgctgctcagggtggcgacatcggttcgggactcgcaagactcctcgccgtcaactatgacgcatgcaaatgcatcaacatcaactacatgcctgccgttgcaccaccagaggacgctccggagcggcaccagatcaaaccgcacgaggaggatgcgctccgacgtgcggacgagtttcagaagacgggcagggggtatgccaacatgcatgcaacgagacccggtacggtcggcatcgtcgtcggtagttcgccggtcgcactgctcgcttggatcgctgaaaagtacctcgcttggaccgatgaggatccgcccctcgacacgatcctcgcaatctgcaccatctggtggatccgcgactcgtacccttcttcaatctgggcctacgccgactttctcgagacgggcatctcggccctgcacaacgacccgaagtacaaacttgacaagaaaccgttcgggttctcgagcttcaaggaggagatcagcgcgactcccgaggcgtgggcgggcaggaacggcaacttgcagttctatcggtaccacgacaagggaggtcactttgcggcgctcgagcagccggaagcgttcgcgcaagacatgcaggattgcttcggcaaaatctggcctctctctcaggagcaaaaatcgtag

SEQ ID NO: 15 - DNA, 29 bp, Artificial Sequence
  Primer
tggatccatgtcgtattcagaccttcccc

SEQ ID NO: 16 - DNA, 35 bp, Artificial Sequence
  Primer
tgctagctcagtaattacctttgtacttctcccac

SEQ ID NO: 17 - DNA, 24 bp, Artificial Sequence
  Primer
agatctatgcccgcccgctcgctc

SEQ ID NO: 18 - DNA, 33 bp, Artificial Sequence
  Primer
tcctaggctacgatttttgctcctgagagagag

SEQ ID NO: 19 - DNA, 21 bp, Artificial Sequence
  Primer
gtggatccatggcgacacaca

SEQ ID NO: 20 - DNA, 22 bp, Artificial Sequence
  Primer
gacctaggctacttctcccaca

SEQ ID NO: 21 - DNA, 23 bp, Artificial Sequence
  Primer
gattaatgatcaatgagcgagca

SEQ ID NO: 22 - DNA, 20 bp, Artificial Sequence
  Primer
gacctaggtcacgacgacag

SEQ ID NO: 23 - DNA, 19 bp, Artificial Sequence
  Primer
gtggatccatggctgccca

SEQ ID NO: 24 - DNA, 18 bp, Artificial Sequence
  Primer
gagctagctcaggcctgg

SEQ ID NO: 25 - DNA, 21 bp, Artificial Sequence
  Primer
gtggatccatggcgacacaca

SEQ ID NO: 26 - DNA, 22 bp, Artificial Sequence
  Primer
gacctaggctacttctcccaca

SEQ ID NO: 27 - DNA, 25 bp, Artificial Sequence
  Primer
cctagggtgtctgtggtatctaagc

SEQ ID NO: 28 - DNA, 17 bp, Artificial Sequence
  Primer
ccgtctccgggagctgc

SEQ ID NO: 29 - DNA, 20 bp, Artificial Sequence
  Primer
catacaaccacacacatcca

SEQ ID NO: 30 - DNA, 21 bp, Artificial Sequence
  Primer
taaatagcttagataccacag

(19) United States
(12) Patent Application Publication
(10) Pub. No.: US 2008/0199912 A1
(43) Pub. Date: Aug. 21, 2008

(54) Methods for Obtaining Optically Active Epoxides and Diols from 2,3-Disubstituted and 2,3-Trisubstituted Epoxides

(75) Inventors: Adriana Leonora Botes, Edgeley (GB); Jeanette Lotter, Edenvale Ridge (ZA); Michel Labuschagne, Pretoria (ZA); Robin Kumar Mitra, Edgeley (GB); Robyn Roth, Sandton (ZA); Nasreen Abrahams, Roodepoort (ZA); Clinton Simpson, Kempton Park (ZA); Christiaan Van Der Westhuyzen, Alberton (ZA)

Correspondence Address: FISH & RICHARDSON P.C., P.O. BOX 1022, MINNEAPOLIS, MN, 55440-1022, US

(73) Assignee: Oxyrane (UK) Ltd., Manchester (GB)

(21) Appl. No.: 11/872,544

(22) Filed: Oct. 15, 2007

Related U.S. Application Data
(63) Continuation of application No. PCT/IB2006/003978, filed on Apr. 14, 2006.

(30) Foreign Application Priority Data
Apr. 14, 2005 (ZA) 2005/03030
Apr. 15, 2005 (ZA) 2005/03083

(51) Int. Cl. C12P 21/00; C12P 17/02; C12P 7/02; C07H 21/02; C12N 9/00; C12N 1/16; C07K 16/00; C07K 14/00

(52) U.S. Cl. 435/70.1; 435/123; 435/156; 536/23.1; 435/183; 435/255.1; 530/387.1; 530/388.1; 530/350

(57) ABSTRACT

The invention provides yeast strains, and polypeptides encoded by genes of such yeast strains, that have enantiospecific internal epoxide hydrolase activity. The invention also features nucleic acid molecules encoding such polypeptides, vectors containing such nucleic acid molecules, and cells containing such vectors. Also embraced by the invention are methods for obtaining optically active internal epoxides and corresponding optically active internal diols.

This application claims priority of South African Provisional Application No. 2005/03030, filed Apr. 14, 2005, and South African Provisional Application No. 2005/03083, filed Apr. 15, 2005. The disclosures of South African Provisional Application Nos. 2005/03030 and 2005/03083 are incorporated herein by reference in their entirety.

### TECHNICAL FIELD

This invention relates to biocatalytic reactions, and more particularly to the use of enantiomer-selective hydrolases to obtain optically active epoxides and diols.

### BACKGROUND

Optically active epoxides and diols (e.g., vicinal diols) are versatile fine chemical intermediates for use in the production of pharmaceuticals, agrochemicals, ferro-electric liquid crystals and flavors and fragrances. Epoxides are highly reactive electrophiles because of the strain inherent in the three-membered ring and the electronegativity of the oxygen. Epoxides react readily with various O-, N-, S-, and C-nucleophiles, acids, bases, reducing and oxidizing agents, allowing access to bifunctional molecules. Diols (e.g., vicinal diols), employed as their highly reactive cyclic sulfites and sulfates, act like epoxide-like synthons with a broad range of nucleophiles. The possibility of double nucleophilic displacement reactions with amidines and azide, allow access to dihydroimidazole derivatives, aziridines, diamines and diazides. Since enantiopure epoxides and diols can be stereospecifically inter-converted, they can be regarded as synthetic equivalents.

Optically active 2,3-di, tri- and tetra-substituted epoxides and their corresponding diols have considerable synthetic potential for the production of a wide range of bioactive compounds. Cis-2,3-disubstituted epoxides that are bioactive compounds include, for example, insect pheromones, antibiotics such as fosfomycin and natural compounds such as C18 epoxy fatty acids that are involved, e.g., in plant defense mechanisms against, for example, rice blast disease. Enantiopure cis-2,3-epoxides (e.g., indene oxide) are also valuable building blocks in the synthesis of pharmaceuticals (e.g. anti-retrovirals and potassium channel opener drugs). Similarly, trans-2,3-epoxides are encountered as sex attractants in insects, and serve as building blocks for pharmaceuticals such as Diltiazem and Taxol. Tri-substituted epoxides are also useful for the synthesis of a wide range of pharmaceuticals and natural products. Derivatives of 6,7-epoxygeranyl alcohol or the esters (for example 6,7-epoxy-3,7-dimethyl-2-octene-1-yl phenylcarbamate) are useful chiral intermediates in the synthesis of insect juvenile hormone analogues and various pharmaceuticals. For example, 2,3-epoxylinalyl acetate can be used in the synthesis of aroma compounds (Orru et al., 1999). Methylene-interrupted bis-epoxides are biosynthetic and bio-mimetic precursors to chiral substituted tetrahydrofurans, which feature in many biologically potent natural products (Capon and Barrow, 1998).

Epoxide hydrolases (ec 3.3.2.3) are hydrolytic enzymes that convert epoxides to vicinal diols by ring-opening of the epoxide with water. Epoxide hydrolases are present in mammals, plants, insects and microorganisms.

### SUMMARY

The invention is based in part on the surprising discovery by the inventors that certain microorganisms express epoxide hydrolases with high enantioselectivity. These microorganisms and the yeast enantioselective 2,3-di- and 2,3-tri-substituted epoxide hydrolases polypeptides of the invention selectively hydrolyse specific enantiomers of 2,3-di- and/or 2,3-tri-substituted epoxides. The genomes of the microorganisms encode polypeptides having highly enantioselective 2,3-di- or 2,3-tri-substituted epoxide hydrolase activity. These 2,3-di- or tri-substituted epoxides are for convenience referred to herein as “internal epoxides” (“IE”) and the term “internal diols” (“ID”) refers to the diols that are the products of the epoxide hydrolase-mediated hydrolysis of the IE. These ID are generally internal vicinal diols (IVD) (see, for example, the compounds represented by general formulae (IV), (V), (VI), and (IX) below). Compounds having the general formula (IX) (and which are IVD), are the products of the YEIH-mediated hydrolyis of compounds having the general formula (VII). However, after formation, compounds with general formula (IX) undergo spontaneous ring closure to form compounds with general formula (VIII), which are ID but not IVD. Thus, while the products of the YEIH-mediated hydrolysis of IE are generally IVD, because of this spontaneous ring closure, IVD are not always recoverable from the appropriate biotransformations. In these cases, the recoverable compounds will nevertheless be ID. The rate at which the spontaneous ring closure occurs depends on the R groups on the IE. With certain R groups it may be possible to recover compounds with general formula (IX) from the appropriate biotransformations by limiting the time of the incubations involved.

The yeast enantioselective hydrolases of the invention having the above described activity on IE are referred to herein as yeast enantioselective internal epoxide hydrolases (YEIH).

More specifically, the invention provides a process for obtaining an optically active IE and/or an optically active ID, which process includes the steps of: providing an enantiomeric mixture of a internal epoxide (IE); creating a reaction mixture by adding to the enantiomeric mixture a polypeptide, or a functional fragment thereof, having enantioselective internal epoxide hydrolase activity, the polypeptide being a polypeptide encoded by a gene of a yeast cell or a gene derived from a yeast cell; incubating the reaction mixture; and recovering from the reaction mixture: (a) an enantiopure, or a substantially enantiopure, ID; (b) an enantiopure, or a substantially enantiopure, IE; or (c) an enantiopure, or a substantially enantiopure, ID and an enantiopure, or a substantially enantiopure, IE.

Another aspect of the invention is a process for obtaining an optically active IE and/or an optically active ID, which process includes the steps of: providing an enantiomeric mixture of a internal epoxide; creating a reaction mixture by adding to the enantiomeric mixture a cell (e.g., a yeast cell) comprising a nucleic acid encoding, and capable of expressing, a polypeptide having enantioselective internal epoxide hydrolase activity; incubating the reaction mixture; and recovering from the reaction mixture: (a) an enantiopure, or a substantially enantiopure, ID; (b) an enantiopure, or a substantially enantiopure, IE; or (c) an enantiopure, or a substantially enantiopure, ID and an enantiopure, or a substantially enantiopure, IE. The polypeptide can be a polypeptide encoded by a gene of a yeast cell.

In both of the above processes, the incubation can result in the selective production of an ID having the chirality of the enantiomer for which the epoxide hydrolase has selective activity and/or the selective enrichment, relative to the total amount of both enantiomers of the ID in the mixture, of the ID enantiomer for which the epoxide does not have selective activity.

The following embodiments apply to both of the above processes. The polypeptide can be encoded by an endogenous gene of the cell or the cell can be a recombinant cell, the polypeptide being encoded by a nucleic acid sequence with which the cell is transformed. The nucleic acid sequence can be an exogenous nucleic acid sequence, a heterologous nucleic acid sequence, or a homologous nucleic acid sequence. The polypeptide can be a full-length YEIH or a functional fragment of a full length YEIH. Moreover, both processes can be carried out at a pH from 5 to 10. They can be carried out at a temperature of 0° C. to 60° C. In the processes, the concentration of the IE (e.g., at the start of reaction) can be at least equal to the solubility of the internal epoxide in water, i.e., it can be at least equal to the soluble concentration of the IE in water.

In both processes, the IE is a compound of the general formula (A) (as exemplified by general formulae (I), (II), (III) and (VII) below) and the ID produced by the process is a compound of the general formula (B) (as exemplified by general formulae (IV), (V), (VI), (VIII), and (IX) below).

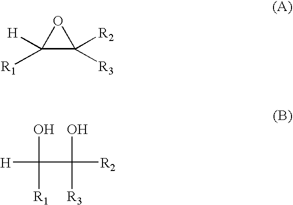

For the IE with general formula (A), R₁ is not H and: (a) where R₂ is H and R₃ is not H, the IE is a cis-2,3-disubstituted epoxide; (b) where R₂ is not H and R₃ is H, the IE is a trans-2,3-disubstituted epoxide; and (c) where neither R₂ nor R₃ are H, the IE is a tri-substituted epoxide.

The 2,3 disubstituted epoxide may be a compound of the general formula (I) and/or (II) and the 2,3-trisubstituted epoxide may be of general formula (III). The ID produced by the processes of the invention can be a compound of the general formula (IV) and/or (V) or (VI).

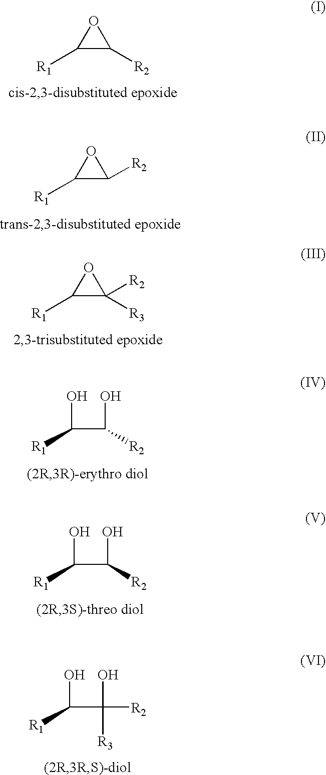

The process may include enantioselective hydrolysis of methylene interrupted bis-epoxides and derivatives thereof, such as diepoxyfatty acids, of the general formula (VII) to the corresponding tetrahydrofuran diols of general formula (VII) and/or tetraols of general formula (IX).

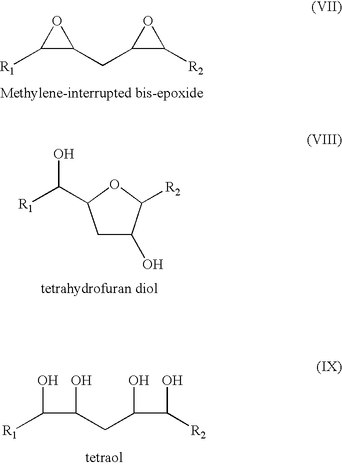

In all of compounds I-IX,

R₁, R₂ and R₃ are, independently of each other, selected from the group consisting of a variably substituted straight-chain or branched alkyl group, a variably substituted straight-chain or branched alkenyl group, a variably substituted straight-chain or branched alkynyl group, a variably substituted cycloalkyl group as well as cycloalkenyl groups, a variably substituted aryl group, a variably substituted aryl-alkyl group, a variably substituted heterocyclic group, a variably substituted straight-chain or branched alkoxy group, a variably substituted straight-chain or branched alkenyloxy group, a variably substituted aryloxy group, a variably substituted aryl-alkyloxy group, a variably substituted alkylthio group, a variably substituted alkoxycarbonyl group, a variably substituted straight chain or branched alkylamino or alkenyl-amino group, a variably substituted arylamino or arylalkylamino group, a variably substituted carbamoyl group, a variably substituted acyl group, and a functional group.

Moreover, in the processes, the enantiomeric mixture can be a racemic mixture or a mixture of any ratio of amounts of the enantiomers. The processes can include adding to the reaction mixture water and at least one water-immiscible solvent, including, for example, toluene, 1,1,2-trichlorotrifluoroethane, methyl tert-butyl ether, methyl isobutyl ketone, dibutyl-o-phtalate, aliphatic alcohols containing 6 to 9 carbon atoms or aliphatic hydrocarbons containing 6 to 16 carbon atoms. Alternatively, or in addition, the processes can include adding to the reaction mixture water and at least one water-miscible organic solvent, for example, acetone, methanol, ethanol, propanol, isopropanol, acetonitrile, dimethylsulfoxide, N,N-dimethylformamide, or N-methylpyrrolidine. In addition, or alternatively, one or more surfactants, one or more cyclodextrins, or one or more phase-transfer catalysts can be added to the reaction mixtures. Both processes can include stopping the reaction when one enantiomer of the epoxide and/or vicinal diol is in excess compared to the other enantiomer of the epoxide and/or vicinal diol. Furthermore, the processes can include recovering continuously during the reaction the optically active epoxide and/or the optically active vicinal diol produced by the reaction directly from the reaction mixture.

In both processes the yeast cell can be of one of the following exemplary genera: Arxula, Brettanomyces, Bullera, Bulleromyces, Candida, Cryptococcus, Debaryomyces, Dekkera, Exophiala, Geotrichum, Hormonema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, or Yarrowia.

Moreover, in the processes, the yeast cell can be of one of the following exemplary species: Arxula adeninivorans, Arxula terrestris, Brettanomyces bruxellensis, Brettanomyces naardenensis, Brettanomyces anomalus, Brettanomyces species (e.g. NCYC 3151), Bullera dendrophila, Bulleromyces albus, Candida albicans, Candida fabianii, Candida glabrata, Candida haemulonii, Candida intermedia, Candida magnoliae, Candida parapsilosis, Candida rugosa, Candida tenuis, Candida tropicalis, Candida famata, Candida kruisei, Candida sp. (new) rel to C. sorbophila, Cryptococcus albidus, Cryptococcus amylolentus, Cryptococcus bhutanensis, Cryptococcus curvatus, Cryptococcus gastricus, Cryptococcus humicola, Cryptococcus hungaricus, Cryptococcus laurentii, Cryptococcus luteolus, Cryptococcus macerans, Cryptococcus podzolicus, Cryptococcus terreus, Cryptococcus macerans, Debaryomyces hansenii, Dekkera anomala, Exophiala dermatitidis, Geotrichum species (e.g. UOFS Y-0111), Hormonema species (e.g. NCYC 3171), Issatchenkia occidentalis, Kluyveromyces marxianus, Lipomyces species (e.g. UOFS Y-2159), Lipomyces tetrasporus, Mastigomyces philipporii, Myxozyma melibiosi, Pichia anomala, Pichia finlandica, Pichia guillermondii, Pichia haplophila, Rhodosporidium lusitaniae, Rhodosporidium paludigenum, Rhodosporidium sphaerocarpum, Rhodosporidium toruloides, Rhodosporidium paludigenum, Rhodotorula araucariae, Rhodotorula glutinis, Rhodotorula minuta, Rhodotorula minuta var. minuta, Rhodotorula mucilaginosa, Rhodotorula philyla, Rhodotorula rubra, Rhodotorula species (e.g. UOFS Y-2042), Rhodotorula species (e.g. UOFS Y-0448), Rhodotorula species (e.g. NCYC 3193), Rhodotorula species (e.g. UOFS Y-0139), Rhodotorula species (e.g. UOFS Y-0560), Rhodotorula aurantiaca, Rhodotorula species (e.g. NCYC 3224), Rhodotorula sp. “mucilaginosa”, Sporidiobolus salmonicolor, Sporobolomyces holsaticus, Sporobolomyces roseus, Sporobolomyces tsugae, Trichosporon beigelii, Trichosporon cutaneum var. cutaneum, Trichosporon delbrueckii, Trichosporon jirovecii, Trichosporon mucoides, Trichosporon ovoides, Trichosporon pullulans, Trichosporon species (e.g. UOFS Y-0861), Trichosporon species (e.g. UOFS Y-1615), Trichosporon species (e.g. UOFS Y-0451), Trichosporon species (e.g. NCYC 3212), Trichosporon species (e.g. UOFS Y-0449), Trichosporon species (e.g. NCYC 3211), Trichosporon species (e.g. UOFS Y-2113), Trichosporon species (e.g. NCYC 3210), Trichosporon monliliforme, Trichosporon montevideense, Wingea robertsiae, or Yarrowia lipolytica.

The yeast cell can also be of any of the other genera, species, or strains disclosed herein.

Another aspect of the invention is a method for producing a polypeptide, which process includes the steps of: providing a cell comprising a nucleic acid encoding and capable of expressing a polypeptide that has enantioselective IE hydrolase activity; and culturing the cell. The method can further involve recovering the polypeptide from the culture. Recovering the polypeptide from the culture includes, for example, recovering it from the medium in which the cell was cultured or recovering it from the cell per se. The cell can be a yeast cell. The polypeptide can be encoded by an endogenous gene of the cell or the cell can be a recombinant cell, the polypeptide being encoded by a nucleic acid sequence with which the cell is transformed. The nucleic acid sequence can be an exogenous nucleic acid sequence, a heterologous nucleic acid sequence, or a homologous nucleic acid sequence. The polypeptide can be a full-length YEIH or a functional fragment of a full-length YEIH. The cell can be of any of the yeast genera, species, or strains disclosed herein or any recombinant cell disclosed herein.

The invention also features a crude or pure enzyme preparation which includes a polypeptide having enantioselective IE hydrolase activity. The polypeptide can be one encoded by any of the yeast genera, species, or strains disclosed herein or one encoded by a recombinant cell.

In another aspect, the invention features a substantially pure culture of cells, a substantial number of which comprise a nucleic acid encoding, and are capable of expressing, a polypeptide having enantioselective IE hydrolase activity. The cells can be recombinant cells or cells of any of the yeast genera, species, or strains disclosed herein.

Another embodiment of the invention is an isolated cell, the cell comprising a nucleic acid encoding a polypeptide having enantioselective IE hydrolase activity, the cell being capable of expressing the polypeptide. The cell can be any of those disclosed herein.

The invention also features an isolated DNA that includes: (a) a nucleic acid sequence that encodes a polypeptide that has enantioselective IE hydrolase activity and that hybridizes under highly stringent conditions to the complement of a sequence that can be SEQ ID NO: 8, 9, 10, 11, 12, 13, or 14; or (b) the complement of the nucleic acid sequence. The nucleic acid sequence can encode a polypeptide that includes an amino acid sequence that can be SEQ ID NO: 1, 2, 3, 4, 5, 6, or 7. The nucleic acid sequence can be, for example, one of those with SEQ ID NOs: 8, 9, 10, 11, 12, 13, or 14.

Also provided by the invention is an isolated DNA that includes: (a) a nucleic acid sequence that is at least 55% identical to a sequence that can be SEQ ID NO: 8, 9, 10, 11, 12, 13, or 14; or (b) the complement of the nucleic acid sequence, the nucleic acid sequence encoding a polypeptide that has enantioselective IE hydrolase activity.

Another aspect of the invention is an isolated DNA that includes: (a) a nucleic acid sequence that encodes a polypeptide consisting of an amino acid sequence that is at least 55% identical to a sequence that can be SEQ ID NOs: 1, 2, 3, 4, 5, 6, or 7; or (b) the complement of the nucleic acid sequence, the polypeptide having enantioselective IE hydrolase activity.

Also included in the inventions are: (i) vectors (e.g., those in which the coding sequence is operably linked to a transcriptional regulatory element) containing any of the above DNAs; and (ii) cells (e.g., eukaryotic or prokaryotic cells) containing such vectors.

Also provided by the invention is an isolated polypeptide encoded by any of the above DNAs. The polypeptide can include an amino acid sequence that is at least 55% identical to SEQ ID NOs: 1, 2, 3, 4, 5, 6 or 7, the polypeptide having enantioselective IE hydrolase activity. The polypeptide can also include: (a) a sequence that can be SEQ ID NO: 1, 2, 3, 4, 5, 6, or 7, or a functional fragment of the sequence; or (b) the sequence of (a), but with no more than five conservative substitutions, the polypeptide having enantioselective IE hydrolase activity.

In another embodiment the invention features an isolated antibody (e.g., a polyclonal or a monoclonal antibody) that binds to any of the above-described polypeptides.

As used herein, the term “wild-type” as applied to a nucleic acid or polypeptide refers to a nucleic acid or a polypeptide that occurs in, or is produced by, respectively, a biological organism as that biological organism exists in nature.

The term “heterologous” as applied herein to a nucleic acid in a host cell or a polypeptide produced by a host cell refers to any nucleic acid or polypeptide (e.g., an YEIH polypeptide) that is not derived from a cell of the same species as the host cell. Accordingly, as used herein, “homologous” nucleic acids, or proteins, are those that are occur in, or are produced by, a cell of the same species as the host cell.

The term “exogenous” as used herein with reference to nucleic acid and a particular host cell refers to any nucleic acid that does not occur in (and cannot be obtained from) that particular cell as found in nature. Thus, a non-naturally-occurring nucleic acid is considered to be exogenous to a host cell once introduced into the host cell. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. Nucleic acid that is naturally-occurring can be exogenous to a particular cell. For example, an entire chromosome isolated from a cell of yeast x is an exogenous nucleic acid with respect to a cell of yeast y once that chromosome is introduced into a cell of yeast.

In contrast, the term “endogenous” as used herein with reference to nucleic acids or genes and a particular cell refers to any nucleic acid or gene that does occur in (and can be obtained from) that particular cell as found in nature.

It will be clear from the above that “exogenous” nucleic acids can be “homologous” or “heterologous” nucleic acids. As used herein, “homologous” nucleic acids are those that are derived from a cell of the same species as the host cell and “heterologous” nucleic acids are those that are derived from a species other than that of the host cell.

As an illustration of the above concepts, an expression plasmid encoding a Y. lipolytica YEIH that is transformed into a Y. lipolytica cell is, with respect to that cell, an exogenous nucleic acid. However, the YEIH coding sequence and the YEIH produced by it are homologous with respect to the cell. Similarly, an expression plasmid encoding a potato epoxide hyrdrolase that is transformed into a Y. lipolytica cell is, with respect to that cell, an exogenous nucleic acid. In contrast, however the epoxide hydrolase coding sequence and the epoxide hydrolases produced by it are heterologous with respect to the cell.

The term “biocatalyst” refers herein to any agent (e.g., an epoxide hydrolase, a recombinant Y. lipolytica cell expressing an epoxide hydrolase, or a lysate or cell extract of such a cell) that initiates or modifies the rate of a chemical reaction in a living body, i.e., a biochemical catalyst. Herein, the term “biotransformation” is the chemical conversion of substances (e.g., epoxides) as by the actions of living organisms (e.g., Yarrowia cells), enzymes expressed therefrom, or enzyme preparations thereof.

In contrast, the term “endogenous” as used herein with reference to nucleic acids or genes and a particular cell refers to any nucleic acid or gene that does occur in (and can be obtained from) that particular cell as found in nature.

The alkyl group may be a straight chain or branched alkyl group with 1 to 12 carbon atoms. The alkyl group may be selected from the group consisting of methyl-, ethyl-, propyl-, isopropyl-, butyl-, isobutyl-, s-butyl-, t-butyl-, pent-1-yl-, pent-2-yl-, pent-3-yl-, 2-methylbut-1-yl-, 3-methylbut-1-yl-, 2-methylbut-2-yl-, 3-methylbut-2-yl-, hex-1-yl-, hex-2-yl-, hex-3-yl-, 1-methylpent-1-yl-, 2-methylpent-1-yl-, 3-methylpent-1-yl-, 2-methylpent-2-yl-, 3-methylpent-2-yl-, 4-methylpent-2-yl-, 2-methylpent-3-yl-, 3-methylpent-3-yl-, 2-ethylbut-1-yl-, hept-1-yl-, hept-2-yl-, hept-3-yl-, hept-4-yl-, 1-methylhex-1-yl-, 2-methylhex-1-yl-, 3-methylhex-1-yl-, 4-methylhex-1-yl-, 5-methylhex-1-yl-, 2-methylhex-2-yl-, 3-methylhex-2-yl-, 4-methylhex-2-yl-, 5-methylhex-2-yl-, 2-methylhex-3-yl-, 3-methylhex-3-yl-, 4-methylhex-3-yl-, 5-methylhex-3-yl-, 2-methylhex-4-yl-, 1,1-dimethylpent-1-yl-, 1,2-dimethylpent-1-yl-, 1,3-dimethylpent-1-yl, 1,4-dimethylpent-1-yl-, 2,2-dimethylpent-1-yl-, 2,3-dimethylpent-1-yl-, 2,4-dimethylpent-1-yl-, 2,5-dimethylpent-1-yl-, 3,3-dimethylpent-1-yl-, 3,4-dimethylpent-1-yl-, 3,5-dimethylpent-1-yl-, 4,4-dimethylpent-1-yl-, 4,5-dimethylpent-1-yl-, 5,5-dimethylpent-1-yl-, 2,2-dimethylpent-2-yl-, 2,3-dimethylpent-2-yl-, 2,4-dimethylpent-2-yl-, 3,3-dimethylpent-2-yl-, 3,4-dimethylpent-2-yl-, 2,2-dimethylpent-3-yl-, 2,3-dimethylpent-3-yl-, 2,4-dimethylpent-3-yl-, 2,2-dimethylpent-4-yl-, 2-ethylpent-1-yl-, 3-ethylpent-1-yl-, 1,1,2-trimethylbut-1-yl-, 1,2,2-trimethylbut-1-yl-, 1,2,3-trimethylbut-1-yl-, 2,2,3-trimethylbut-1-yl-, 2,3,3-trimethylbut-1-yl-, 2,3,3-but-2-yl-, 2-isopropylbut-1-yl-, 2-isopropylbut-2-yl-, oct-1-yl-, oct-2-yl-, oct-3-yl-, oct-4-yl-, 2-methylhept-1-yl-, 3-methylhept-1-yl-, 4-methylhept-1-yl-, 5-methylhept-1-yl-, 6-methylhept-1-yl-, 2-methylhept-2-yl-, 3-methylhept-2-yl-, 4-methylhept-2-yl-, 5-methylhept-2-yl-, 6-methylhept-2-yl-, 2-methylhept-3-yl-, 3-methylhept-3-yl-, 4-methylhept-3-yl-, 5-methylhept-3-yl-, 6-methylhept-3-yl-, 2-methylhept-4-yl-, 3-methylhept-4-yl-, 4-methylhept-4-yl-, 2,2-dimethylhex-1-yl-, 2,3-dimethylhex-1-yl-, 2,4-dimethylhex-1-yl-, 2,5-dimethylhex-1-yl-, 3,3-dimethylhex-1-yl-, 3,4-dimethylhex-1-yl-, 3,5-dimethylhex-1-yl-, 4,4-dimethylhex-1-yl-, 4,5-dimethylhex-1-yl-, 5,5-dimethylhex-1-yl-, 2,3-dimethylhex-2-yl-, 2,4-dimethylhex-2-yl-, 2,5-dimethylhex-2-yl-, 3,3-dimethylhex-2-yl-, 3,4-dimethylhex-2-yl-, 3,5-dimethylhex-2-yl-, 4,4-dimethylhex-2-yl-, 4,5-dimethylhex-2-yl-, 5,5-dimethylhex-2-yl-, 2,2-dimethylhex-3-yl-, 2,3-dimethylhex-3-yl-, 2,4-dimethylhex-3-yl-, 2,5-dimethylhex-3-yl-, 3,3-dimethylhex-3-yl-, 3,4-dimethylhex-3-yl-, 3,5-dimethylhex-3-yl-, 4,4-dimethylhex-3-yl-, 4,5-dimethylhex-3-yl-, 5,5-dimethylhex-3-yl-, 2,2,3-trimethylpent-1-yl-, 2,2,4-trimethylpent-1-yl-, 2,3,3-trimethylpent-1-yl-, 2,3,4-trimethylpent-1-yl-, 3,3,4-trimethylpent-1-yl-, 3,4,4-trimethylpent-1-yl-, 2,4,4-trimethylpent-1-yl-, 2,3,3-trimethylpent-2-yl-, 2,3,4-trimethylpent-2-yl-, 3,3,4-trimethylpent-2-yl-, 3,4,4-trimethylpent-2-yl-, 2,4,4-trimethylpent-2-yl-, 2,2,3-trimethylpent-3-yl-, 2-methyl-3-ethylpen-1-yl-, 3-ethyl-3-methylpent-1-yl-, 3-ethyl-4-methylpent-1-yl-, (3-methylhex-3-yl)methyl-, (4-methylhex-3-yl)methyl-, (5-methylhex-3-yl)methyl-, (2-methylhex-2-yl)methyl-, 2-methyl-3-ethylpent-2-yl-, 3-ethyl-3-methylpent-2-yl-, 3-ethyl-4-methylpent-2-yl-, 2-methyl-2-ethylpent-3-yl-, 2-methyl-3-ethylpent-3-yl-, 2,2,3,3-tetramethylbut-1-yl-, 2-ethyl-3,3-dimethylbut-2-ly, 2-isopropyl-3-methylbut-2-yl-, (3-ethylpent-3-yl)methyl-, (2,3-dimethylpent-3-yl)methyl-, (2,4-dimethylpent-3-yl)methyl-, non-1-yl-, non-2-yl-, non-3-yl-, non-4-yl-, non-5-yl-, 2-methyloct-1-yl, 3-methyloct-1-yl-, 4-methyloct-1-yl-, 5-methyloct-1-yl-, 6-methyloct-1-yl-, 7-methyloct-1-yl-, 2-methyloct-2-yl, 3-methyloct-2-yl-, 4-methyloct-2-yl-, 5-methyloct-2-yl-, 6-methyloct-2-yl-, 7-methyloct-2-yl-, 2-methyloct-3-yl, 3-methyloct-3-yl-, 4-methyloct-3-yl-, 5-methyloct-3-yl-, 6-methyloct-3-yl-, 7-methyloct-3-yl-, 2-methyloct-4-yl, 3-methyloct-4-yl-, 4-methyloct-4-yl-, 5-methyloct-4-yl-, 6-methyloct-4-yl-, 7-methyloct-4-yl-, 2,2-dimethylhept-1-yl-, 2,3-dimethylhept-1-yl-, 2,4-dimethylhept-1-yl-, 2,5-dimethylhept-1-yl-, 2,6-dimethylhept-1-yl-, 3,3-dimethylhept-1-yl-, 3,4-dimethylhept-1-yl-, 3,5-dimethylhept-1-yl-, 3,6-dimethylhept-1-yl-, 4,4-dimethylhept-1-yl-, 4,5-dimethylhept-1-yl-, 4,6-dimethylhept-1-yl-, 5,5-dimethylhept-1-yl-, 5,6-dimethylhept-1-yl-, 6,6-dimethylhept-1-yl-, 2,3-dimethylhept-2-yl-, 2,4-dimethylhept-2-yl-, 2,5-dimethylhept-2-yl-, 2,6-dimethylhept-2-yl-, 3,3-dimethylhept-2-yl-, 3,4-dimethylhept-2-yl-, 3,5-dimethylhept-2-yl-, 3,6-dimethylhept-2-yl-, 4,4-dimethylhept-2-yl-, 4,5-dimethylhept-2-yl-, 4,6-dimethylhept-2-yl-, 5,5-dimethylhept-2-yl-, 5,6-dimethylhept-2-yl-, 6,6-dimethylhept-2-yl-, 2,2-dimethylhept-3-yl-, 2,3-dimethylhept-3-yl-, 2,4-dimethylhept-3-yl-, 2,5-dimethylhept-3-yl-, 2,6-dimethylhept-3-yl-, 3,4-dimethylhept-3-yl-, 3,5-dimethylhept-3-yl-, 3,6-dimethylhept-3-yl-, 4,4-dimethylhept-3-yl-, 4,5-dimethylhept-3-yl-, 4,6-dimethylhept-3-yl-, 5,5-dimethylhept-3-yl-, 5,6-dimethylhept-3-yl-, 6,6-dimethylhept-3-yl-, 3-ethylhept-1-yl-, 3-ethylhept-1-yl-, 4-ethylhept-1-yl-, 3-ethylhept-2-yl-, 4-ethylhept-2-yl-, 5-ethylhept-2-yl-, 3-ethylhept-3-yl-, 4-ethylhept-3-yl-, 5-ethylhept-3-yl-, 3-ethylhept-4-yl-, 4-ethylhept-4-yl-, 2,2,3-trimethylhex-1-yl-, 2,2,4-trimethylhex-1-yl-, 2,2,5-trimethylhex-1-yl-, 2,3,3-trimethylhex-1-yl-, 2,3,4-trimethylhex-1-yl-, 2,3,5-trimethylhex-1-yl-, 2,4,4-trimethylhex-1-yl-, 2,4,5-trimethylhex-1-yl-, 2,5,5-trimethylhex-1-yl-, 3,3,4-trimethylhex-1-yl-, 3,3,5-trimethylhex-1-yl-, 4,4,5-trimethylhex-1-yl-, 4,5,5-trimethylhex-1-yl-, 2,3,3-trimethylhex-2-yl-, 2,3,4-trimethylhex-2-yl-, 2,3,5-trimethylhex-2-yl-, 2,4,4-trimethylhex-2-yl-, 2,4,5-trimethylhex-2-yl-, 2,5,5-trimethylhex-2-yl-, 3,3,4-trimethylhex-2-yl-, 3,3,5-trimethylhex-2-yl-, 3,4,4-trimethylhex-2-yl-, 3,4,5-trimethylhex-2-yl-, 3,5,5-trimethylhex-2-yl-, 4,4,5-trimethylhex-2-yl-, 4,5,5-trimethylhex-2-yl-, 2,2,3-trimethylhex-3-yl-, 2,2,4-trimethylhex-3-yl-, 2,2,5-trimethylhex-3-yl-, 2,3,4-trimethylhex-3-yl-, 2,3,5-trimethylhex-3-yl-, 2,4,4-trimethylhex-3-yl-, 2,4,5-trimethylhex-3-yl-, 2,5,5-trimethylhex-3-yl, 4,4,5-trimethylhex-3-yl-, 4,5,5-trimethylhex-3-yl-, (2-methylhex-3-yl)methyl-, 3-ethyl-2-methylhex-1-yl-, 3-ethyl-3-methylhex-1-yl-, 3-ethyl-4-methylhex-1-yl-, 3-ethyl-5-methylhex-1-yl-, 4-ethyl-2-methylhex-1-yl-, 4-ethyl-3-methylhex-1-yl-, 4-ethyl-4-methylhex-1-yl-, 4-ethyl-5-methylhex-1-yl-, (2-methylhex-1-yl)methyl-, (3-methylhex-1-yl)methyl-, (4-methylhex-1-yl)methyl-, (5-methylhex-1-yl)methyl-, (6-methylhex-1-yl)methyl-, 3-isopropylhex-1-yl-, 4-ethyl-5-methylhex-1-yl-, 3-ethyl-3-methylhex-2-yl-, 3-ethyl-4-methylhex-2-yl-, 3-ethyl-5-methylhex-2-yl-, 4-ethyl-2-methylhex-2-yl-, 4-ethyl-3-methylhex-2-yl-, 4-ethyl-4-methylhex-2-yl-, 4-ethyl-5-methylhex-2-yl-, 3-isopropylhex-2-yl-, 4-ethyl-5-methylhex-2-yl-, 3-ethyl-2-methylhex-3-yl-, 3-ethyl-4-methylhex-3-yl-, 3-ethyl-5-methylhex-3-yl-, 4-ethyl-2-methylhex-3-yl-, 4-ethyl-3-methylhex-3-yl-, 4-ethyl-4-methylhex-3-yl-, 4-ethyl-5-methylhex-3-yl-, 4-isopropylhex-1-yl-, 2,2,3,3-tetramethylpent-1-yl-, 2,2,3,4-tetramethylpent-1-yl-, 2,2,4,4-tetramethylpent-1-yl-, 2,3,3,4-tetramethylpent-1-yl-, 2,3,4,4-tetramethylpent-1-yl-, 2,3,4,4-tetramethylpent-1-yl-, 3,3,4,4-tetramethylpent-1-yl, 2,3,3,4-tetramethylpent-2-yl-, 2,3,4,4-tetramethylpent-2-yl-, 2,3,4,4-tetramethylpent-2-yl-, 3,3,4,4-tetramethylpent-2-yl-, 2,2,3,4-tetramethylpent-3-yl-, 2,2,4,4-tetramethylpent-3-yl-, 2,3,4,4-tetramethylpent-3-yl-, 2,3,4,4-tetramethylpent-3-yl-, (3-ethylhex-3-yl)methyl-, (4-ethylhex-3-yl)methyl-, (5-methyl hept-3-yl)methyl-, 2,4-dimethyl-3-ethylpent-1-yl-, 3,4-dimethyl-3-ethylpent-1-yl-, 4,4-dimethyl-3-ethylpent-1-yl-, 2-ethyl-2-methylhex-1-yl-, 3-ethyl-2-methylhex-1-yl-, 4-ethyl-2-methylhex-1-yl-, 2-ethyl-3-methylhex-1-yl-, 2-ethyl-4-methylhex-1-yl-, 3-ethyl-3-methylhex-1-yl-, 3-ethyl-4-methylhex-1-yl-, 3-ethyl-5-methylhex-1-yl-, 4-ethyl-3-methylhex-1-yl-, 4-ethyl-4-methylhex-1-yl-, 4-ethyl-5-methylhex-1-yl-, and the like from dec-1-yl-, dec-2-yl-, dec-3-yl-, dec-4-yl-, dec-5-yl-, dec-6-yl-, undec-1-yl-, undec-2-yl-, undec-3-yl-, undec-4-yl-, undec-5-yl-, undec-6-yl-, undec-7-yl-, dodec-1-yl, dodec-2-yl, dodec-3-yl, dodec-4-yl, dodec-5-yl, dodec-6-yl groups. Preferably, the alkyl group is a straight chain or branched alkyl group with 1 to 8 carbon atoms.

The alkenyl group can be a straight chain or branched alkenyl group having 2-12 carbon atoms. The alkenyl group can be selected from the group consisting of: vinyl-; allyl-; ?-methallyl-; ?-methallyl-; 1-propenyl-; isopropenyl-; 1-butenyl-; 2-butenyl-; 3-butenyl; 1-buten-2-yl-; 1-buten-3-yl-; 1-methyl-1-propenyl-; 2-methyl-1-propenyl-; 1-pentenyl-; 2-pentenyl-; 3-pentenyl-; 4-pentenyl-; 1-penten-2-yl-; 1-penten-3-yl-; 2-methyl-1-butenyl-; 1-hexenyl-; 2-hexenyl-; 3-hexenyl-; 4-hexenyl-; 5-hexenyl-; 1-heptenyl-; 2-heptenyl-; 3-heptenyl-; 4-heptenyl-; 5-heptenyl-; 6-heptenyl-; 1-octenyl-; 2-octenyl-; 3-octenyl-; 4-octenyl-; 5-octenyl-; 6-octenyl-; 7-octenyl-; 1-nonenyl-; 2-nonenyl-; 3-nonenyl-; 4-nonenyl-; 5-nonenyl-; 6-nonenyl-; 7-nonenyl-; 8-nonenyl-; 1-decenyl-; 2-decenyl-; 3-decenyl-; 4-decenyl-; 5-decenyl-; 6-decenyl-; 7-decenyl-; 8-decenyl-; 9-decenyl-; 1-undecenyl; 2-undecenyl; 3-undecenyl; 4-undecenyl; 5-undecenyl; 6-undecenyl; 7-undecenyl; 8-undecenyl; 9-undecenyl; 10-undecenyl; 1-dodecenyl; 2-dodecenyl; 3-dodecenyl; 4-dodecenyl; 5-dodecenyl; 6-dodecenyl; 7-dodecenyl; 8-dodecenyl; 9-dodecenyl; 10-dodecenyl; 11-dodecenyl groups; and related branched isomers. Preferably, the alkenyl group is a straight chain or branched alkenyl group with 2 to 8 carbon atoms.

The alkynyl group can be a straight chain or branched alkynyl group having 2-12 carbon atoms. The alkynyl group can be selected from the group consisting of: ethynyl-; 1-propynyl-; 2-propynyl-; 1-butynyl-; 2-butynyl-; 3-butynyl-; 1-pentynyl-; 2-pentynyl-; 3-pentynyl-; 4-pentynyl-; 1-hexynyl-; 2-hexynyl-; 3-hexynyl-; 4-hexynyl-; 5-hexynyl-; 1-heptynyl-; 2-heptynyl-; 3-heptynyl-; 4-heptynyl-; 5-heptynyl-; 6-heptynyl-; 1-octynyl-; 2-octynyl-; 3-octynyl-; 4-octynyl-; 5-octynyl-; 6-octynyl-; 7-octynyl-; 1-nonynyl-; 2-nonynyl-; 3-nonynyl-; 4-nonynyl-; 5-nonynyl-; 6-nonynyl-; 7-nonynyl-; 8-nonynyl-; 1-decynyl-; 2-decynyl-; 3-decynyl-; 4-decynyl-; 5-decynyl-; 6-decynyl-; 7-decynyl-; 8-decynyl-; 9-decynyl-; 1-undecynyl-; 2-undecynyl-; 3-undecynyl-; 4-undecynyl-; 5-undecynyl-; 6-undecynyl-; 7-undecynyl-; 8-undecynyl-; 9-undecynyl-; 10-undecynyl-; 1-dodecynyl-; 2-dodecynyl-; 3-dodecynyl-; 4-dodecynyl-; 5-dodecynyl-; 6-dodecynyl-; 7-dodecynyl-; 8-dodecynyl-; 9-dodecynyl-; 10-dodecynyl-; 11-dodecynyl-groups; and related branched isomers. Preferably, the alkynyl group is a straight chain or branched alkenyl group with 2 to 8 carbon atoms.

The cycloalkyl group can be cycloalkyl groups with 3 to 10 carbon atoms. The cycloalkyl group can be selected from the group consisting of: cyclopropyl-; cyclobutyl-; cyclopentyl-; cyclohexyl-; cycloheptyl-; and cyclooctyl-groups. These groups can be variably substituted at any position(s) around the ring. Preferably, the cycloalkyl group is a cycloalkyl group with 5 to 7 carbon atoms.

The cycloalkenyl group can be cycloalkenyl groups with 3 to 10 carbon atoms. The cycloalkenyl group can be selected from the group consisting of cyclobutenyl-; cyclopentenyl-; cyclohexenyl-; cycloheptenyl-; and cyclooctenyl-groups that can variably be substituted at any position(s) around the ring. Preferably, the cycloalkenyl group is a cycloalkenyl group with 5 to 7 carbon atoms.

The aryl group can be selected from the group consisting of phenyl; biphenyl; naphtyl; anthracenyl groups; and the like. Preferably, the aryl group is a phenyl group.

The aryl-alkyl group can be a group with 7 to 18 carbons. The aryl alkyl group can be selected from the group consisting of: benzyl-; 1-methylbenzyl-; 2-phenylethyl-; 3-phenylpropyl-; 4-phenylbutyl-; 5-phenylpentyl-; 6-phenylhexyl-; 1-naphtylmethyl; and 2-(1-naphtyl)-ethyl groups; and the like. Preferably, the aryl alkyl group is an aryl alkyl group with 7 to 12 carbon atoms.

The heterocyclic group can include 5- to 10-membered heterocyclic groups containing nitrogen, oxygen, or sulfur. The heterocyclic ring can be fused with a cyclic or aromatic ring having 3 to 7 carbon atoms such as benzene; cyclopropyl; cyclobutane; cyclopentane; and cyclohexane ring systems. Preferably the heterocyclic ring has 5 or 6 carbon atoms. The heterocyclic ring can be selected from the group consisting of: furyl-; dihydrofuranyl-; tetrahydrofuranyl-; dioxolanyl-; oxazolyl-; dihydrooxazolyl-; oxazolidinyl-; isoxazolyl-; dihydroisoxazolyl-; isoxazolidinyl-; oxathiolanyl-; thienyl-; tetrahydrothienyl-; dithiolanyl-; thiazolyl-; dihydrothiazolyl-; thiazolidinyl-; isothiazolyl-; dihydroisothiazolyl-; isothiazolidinyl-; pyrrolyl-; dihydropyrrolyl-; pyrrolidinyl-; pyrazolyl-; dihydropyrazolyl-; pyrazolidinyl-; imidazolyl-; dihydroimidazolyl-; imidazolidinyl-; triazolyl-; dihydrotriazolyl-; triazolidinyl-; tetrazolyl-; dihydrotetrazolyl-; tetrazolidinyl-; pyridyl-; dihydropyridyl-; piperidinyl-; morpholinyl-; dioxanyl-; oxathianyl-; trioxanyl-; thiomorpholinyl-; pyridazinyl-; dihydropyridazinyl-; tetrahydropyridazinyl-; hexahydropyridazinyl-; pyrimidinyl-; dihydropyrimadinyl-; tetrahydropyrimadinyl-; hexahydropyrimadinyl-; pyrazinyl-; piperazinyl-; pyranyl-; dihydropyranyl-; tetrahydropyranyl-; thiopyranyl-; dihydrothiopyranyl-; tetrahydrothiopyranyl-; dithianyl-; purinyl-; pyrimidinyl-; pyrrolizinyl-; pyrrolizidinyl; indolyl-; dihydroindolyl-; isoindolyl-; indolizinyl-; indolizidinyl-; quinolyl-; dihydroquinolyl-; tetrahydroquinolyl-; isoquinolyl-; dihydroquinolyl-; tetrahydroquinolyl-; quinolizinyl-; quinolizidinyl-; phenanthrolinyl-; chromenyl-; chromanyl-; isochromenyl-; isochromanyl-; benzofuranyl-; and carbazolyl-groups; and the like.

The alkoxy group can be a straight chain or branched alkoxy group having 2-12 carbon atoms such as methoxy; ethoxy; propyloxy; isopropyloxy; butyloxy; isobutyloxy; tert-butyloxy; pentyloxy; hexyloxy; heptyloxy; or octyloxy.

The alkenyloxy group can be a straight chain or branched alkenyloxy group having 2-12 carbon atoms. The alkenyloxy group can be selected from the group consisting of: ethynyloxy-; 1-propynyloxy-; 2-propynyloxy-; 1-butynyloxy-; 2-butynyloxy-; 3-butynyloxy-; 1-pentynyloxy-; 2-pentynyloxy-; 3-pentynyloxy-; 4-pentynyloxy-; 1-hexynyloxy-; 2-hexynyloxy-; 3-hexynyloxy-; 4-hexynyloxy-; 5-hexynyloxy-; 1-heptynyloxy-; 2-heptynyloxy-; 3-heptynyloxy-; 4-heptynyloxy-; 5-heptynyloxy-; 6-heptynyloxy-; 1-octynyloxy-; 2-octynyloxy-; 3-octynyloxy-; 4-octynyloxy-; 5-octynyloxy-; 6-octynyloxy-; 7-octynyloxy-; 1-nonynyloxy-; 2-nonynyloxy-; 3-nonynyl-oxy-; 4-nonynyloxy-; 5-nonynyloxy-; 6-nonynyloxy-; 7-nonynyloxy-; 8-nonynyloxy-; 1-decynyloxy-; 2-decynyloxy-; 3-decynyloxy-; 4-decynyloxy-; 5-decynyloxy-; 6-decynyloxy-; 7-decynyloxy-; 8-decynyloxy-; 9-decynyloxy-; 1-undecynyloxy-; 2-undecynyloxy-; 3-undecynyloxy-; 4-undecynyloxy-; 5-undecynyloxy-; 6-undecynyloxy-; 7-undecynyloxy-; 8-undecynyloxy-; 9-undecynyloxy-; 10-undecynyloxy-; 1-dodecynyloxy-; 2-dodecynyloxy-; 3-dodecynyloxy-; 4-dodecynyloxy-; 5-dodecynyloxy-; 6-dodecynyloxy-; 7-dodecynyloxy-; 8-dodecynyloxy-; 9-dodecynyloxy-; 10-dodecynyloxy-; and 11-dodecynyloxy-groups; and related branched isomers. Preferably, the alkenyloxy group is a straight chain or branched alkenyloxy groups with 2 to 8 carbon atoms.

The aryloxy group can be an aryloxy group, such as a phenoxy or naphtyloxy group (e.g.: phenoxy; 2-methylphenoxy; 3-methylphenoxy; 4-methylphenoxy; 2-allylphenoxy; 2-chlorophenoxy; 3-chlorophenoxy; 4-chlorophenoxy; 4-methoxyphenoxy; 2-allyloxyphenoxy; naphtyloxy; and the like). The group can optionally be substituted with an alkyl or alkenyl or alkoxy group having 1 to 4 carbon atoms or halogens.

The aryl-alkyloxy group can be benzyloxy or 2-phenylethyloxy.

The alkylamino group can be a straight chain or branched alkylamino group having 2-12 carbon atoms such as: methylamino; ethylamino; propylamino; isopropylamino; butylamino; isobutylamino; tert-butylamino; pentylamino; hexylamino; heptylamino; or octylamino.

The alkenyl-amino group can be a straight chain or branched alkenylamino group having 2-12 carbon atoms. The alkenyl amino group can be selected from the group consisting of: ethynylamino-; 1-propynylamino-; 2-propynylamino-; 1-butynylamino-; 2-butynylamino-; 3-butynylamino-; 1-pentynylamino-; 2-pentynylamino-; 3-pentynylamino-; 4-pentynylamino-; 1-hexynylamino-; 2-hexynylamino-; 3-hexynylamino-; 4-hexynylamino-; 5-hexynylamino-; 1-heptynylamino-; 2-heptynylamino-; 3-heptynylamino-; 4-heptynylamino-; 5-heptynylamino-; 6-heptynylamino-; 1-octynylamino-; 2-octynylamino-; 3-octynylamino-; 4-octynylamino-; 5-octynylamino-; 6-octynylamino-; 7-octynylamino-; 1-nonynylamino-; 2-nonynylamino-; 3-nonynyl-amino; 4-nonynylamino-; 5-nonynylamino-; 6-nonynylamino-; 7-nonynylamino-; 8-nonynylamino-; 1-decynylamino-; 2-decynylamino-; 3-decynylamino-; 4-decynylamino-; 5-decynylamino-; 6-decynylamino-; 7-decynylamino-; 8-decynylamino-; 9-decynylamino-; 1-undecynylamino-; 2-undecynylamino-; 3-undecynylamino-; 4-undecynylamino-; 5-undecynylamino-; 6-undecynylamino-; 7-undecynylamino-; 8-undecynylamino-; 9-undecynylamino-; 10-undecynylamino-; 1-dodecynylamino-; 2-dodecynylamino-; 3-dodecynylamino-; 4-dodecynylamino-; 5-dodecynylamino-; 6-dodecynylamino-; 7-dodecynylamino-; 8-dodecynylamino-; 9-dodecynylamino-; 10-dodecynylamino-; and 11-dodecynylamino-groups; and related branched isomers. Preferably, the alkenyl amino group is a straight chain or branched alkenylamino group with 2 to 8 carbon atoms.

The arylamino group can be an arylamino group such as a phenylamino or naphtylamino group, optionally substituted with an alkyl or alkenyl or alkoxy group having 1 to 4 carbon atoms, or halogens. The arylamino group can be selected from the group consisting of: phenylamino; 2-methylphenylamino; 3-methylphenylamino; 4-methylphen-ylamino; 2-allylphenylamino; 2-chlorophenylamino; 3-chlorophenlamini, 4-chlorophenylamino; 4-methoxyphenylamino; 2-allyloxyphenylamino; naphtylamino; and the like.

The arylalkylamino group can be benzylamino or 2-phenylethylamino.

The alkylthio group can be an alkylthio group having 1 to 8 carbon atoms. The alkylthio group can be selected from the group consisting of: methylthio; ethylthio; propylthio; butylthio; isobutylthio; and pentylthio.

The alkenylthio group can be a straight chain or branched alkenylthio group having 1 to 8 carbon atoms. The alkenylthio group can be selected from the group consisting of: ethynylthio-; 1-propynylthio-; 2-propynylthio-; 1-butynylthio-; 2-butynylthio-; 3-butynylthio-; 1-pentynylathio-; 2-pentynylthio-; 3-pentynylthio-; 4-pentynylthio-; 1-hexynylthio-; 2-hexynylthio-; 3-hexynylthio-; 4-hexynylthio-; 5-hexynylthio-; and the like.

The arylrthio group can be an alkenylthio group having 1 to 8 carbon atoms such as a phenylthio or naphtylthio group, which can optionally be substituted with an alkyl or alkenyl or alkoxy group having 1 to 4 carbon atoms; and also halogens, e.g.: phenylthio; 2-methylphenylthio; 3-methylphenylthio; 4-methylphenylthio; 2-allylphenylthio; 2-chlorophenylthio; 3-chlorophenylamini; 4-chlorophenylthio; 4-methoxyphenylthio; 2-allyloxyphenylthio; naphtylthio; and the like.

The arylalkylthio group can be an alkenylthio group having 1 to 8 carbon atoms such as a benzylthio-group or a 2-phenylethylthio-group.

The alkoxycarbonyl group can be: methoxycarbonyl; ethoxycarbonyl; or the like.

The substituted or unsubstituted carbamoyl group can be: carbamoyl; methylcarbamoyl; dimethylcarbamoyl; diethylcarbamoyl; or the like.

The acyl group can be an acyl group with 1 to 8 carbon atoms such as: formyl; acetyl; propionyl; or benzoyl groups; or the like.

The alkyl, alkenyl, alkynyl, cycloalkyl, aryl, aryl alkyl, heterocyclic, alkoxy, alkenyloxy, aryloxy, aryl-alkyloxy, alkylamino, alkenylamino, arylamino, arylalkylamino, alkylthio, alkenylthio, arylthio, arylalkylthio, alkoxycarbonyl, substituted and unsubstituted carbamoyl and acyl groups mentioned above can optionally be substituted. Examples of substituents include: halogens (F; Cl; Br; I); hydroxyl groups; mercapto groups; carboxylates; nitro groups; cyano groups; substituted or unsubstituted amino groups (including amino, methylamino, dimethylamino, ethylamino, diethylamino, and various protected amines such is tert-butoxycarbonyl- and arylsulfonamido groups); alkoxy groups (having 1 to 8 carbon atoms such as methoxy, ethoxy, propyloxy, isopropyloxy, butyloxy, isobutyloxy, tert-butyloxy, pentyloxy, hexyloxy, heptyloxy or octyloxy); alkenyloxy groups (having 2 to 8 carbon atoms such as a vinyloxy; allyloxy; 3-butenyloxy or 5-hexenyloxy); aryloxy groups (such as a phenoxy or naphtyloxy group which can be optionally substituted with an alkyl or alkenyl or alkoxy group having 1 to 4 carbon atoms; and also halogens, e.g., phenoxy, 2-methylphenoxy, 3-methylphenoxy, 4-methylphenoxy, 2-allylphenoxy, 2-chlorophenoxy, 3-chlorophenoxy, 4-chlorophenoxy, 4-methoxyphenoxy, 2-allyloxyphenoxy, naphtyloxy, and the like); aryl alkyloxy groups (e.g., benzyloxy and 2-phenylethyloxy); alkylthio groups (having 1 to 8 carbon atoms such as methylthio, ethylthio, propylthio, butylthio, isobutylthio, pentylthio); alkoxycarbonyl groups (e.g. methoxycarbonyl, ethoxycarbonyl, and the like); substituted or unsubstituted carbamoyl group (e.g. carbamoyl, methylcarbamoyl, dimethylcarbamoyl, diethylcarbamoyl and the like); acyl groups (with 1 to 8 carbon atoms such as formyl, acetyl, propionyl, or benzoyl groups); and others.

The above-mentioned cycloalkyl, cycloalkenyl, aryl, aryl alkyl, heterocyclic, alkoxy, alkenyloxy, aryloxy, aryl alkyloxy, alkylthio, and alkoxycarbonyl groups can also be substituted with alkyl groups having 1 to 5 carbon atoms, alkenyl groups with 2 to 5 carbon atoms, or haloalkyl groups with 1 to 5 carbon atoms in addition to the substituents specified above.

The number of substituents can be one or more than one. The substituents can be the same or different.

One of the R₁ or R₂ or R₃ groups can be a functional group. The functional group is selected from the group consisting of: halo, pseudohalo, hydroxyl, variably substituted mercapto, variably substituted sulfinyl, variably substituted sulfonyl, carboxylates, variably substituted amino, variably substituted amido, variably substituted ureido, variably substituted carbamoyl, and variably substituted urethano. Pseudohalo is nitro, cyano, azido, cyanato, isocyanato, or isothiocyanato.

R₁ and R₂ together can also be present as a saturated or unsaturated optionally substituted carbocycle or a heterocycle having 5 to 12 carbon atoms, such as: cyclopentane; cyclohexane; cyclohexene; cyclohexadiene; cycloheptane; cyclooctane; cyclononane; or cyclodecane.

As used herein, a “variably substituted” group is a group that is unsubstituted or is substituted with one or more, in another embodiment one to five, in another embodiment one, two or three, substituents.

“Polypeptide” and “protein” are used interchangeably and mean any peptide-linked chain of amino acids, regardless of length or post-translational modification. The invention also features YEIH polypeptides with conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The term “isolated” polypeptide or peptide fragment, as used herein, refers to a polypeptide or a peptide fragment which either has no naturally-occurring counterpart or has been separated or purified from components which naturally accompany it, e.g., microorganism cellular components such as yeast cell cellular components. Typically, the polypeptide or peptide fragment is considered “isolated” when it is at least 70%, by dry weight, free from the proteins and other naturally-occurring organic molecules with which it is naturally associated. Preferably, a preparation of a polypeptide (or peptide fragment thereof) of the invention is at least 80%, more preferably at least 90%, and most preferably at least 99%, by dry weight, the polypeptide (or the peptide fragment thereof), respectively, of the invention. Thus, for example, a preparation of polypeptide X is at least 80%, more preferably at least 90%, and most preferably at least 99%, by dry weight, polypeptide X. Since a polypeptide that is chemically synthesized is, by its nature, separated from the components that naturally accompany it, the synthetic polypeptide is “isolated.”

An isolated polypeptide (or peptide fragment) of the invention can be obtained, for example, by: extraction from a natural source (e.g., from yeast cells); expression of a recombinant nucleic acid encoding the polypeptide; or chemical synthesis. A polypeptide that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

An “isolated DNA” is either (1) a DNA that contains sequence not identical to that of any naturally occurring sequence, or (2), in the context of a DNA with a naturally-occurring sequence (e.g., a cDNA or genomic DNA), a DNA free of at least one of the genes that flank the gene containing the DNA of interest in the genome of the organism in which the gene containing the DNA of interest naturally occurs. The term therefore includes a recombinant DNA incorporated into a vector, into an autonomously replicating plasmid or virus, or into the genomic DNA of a prokaryote or eukaryote. The term also includes a separate molecule such as: a cDNA (e.g., SEQ ID NOs: 8, 9, 10, 11, 12, 13, or 14) where the corresponding genomic DNA has introns and therefore a different sequence; a genomic fragment that lacks at least one of the flanking genes; a fragment of cDNA or genomic DNA produced by polymerase chain reaction (PCR) and that lacks at least one of the flanking genes; a restriction fragment that lacks at least one of the flanking genes; a DNA encoding a non-naturally occurring protein such as a fusion protein, mutein, or fragment of a given protein; and a nucleic acid which is a degenerate variant of a cDNA or a naturally occurring nucleic acid. In addition, it includes a recombinant nucleotide sequence that is part of a hybrid gene, i.e., a gene encoding a non-naturally occurring fusion protein. Also included is a recombinant DNA that includes a portion of any of SEQ ID NOs: 8-14. It will be apparent from the foregoing that isolated DNA does not mean a DNA present among hundreds to millions of other DNA molecules within, for example, cDNA or genomic DNA libraries or genomic DNA restriction digests in, for example, a restriction digest reaction mixture or an electrophoretic gel slice.

As used herein, a “functional fragment” of a YEIH is a fragment of the YEIH that is shorter than the full-length polypeptide and has at least 20% (e.g., at least: 30%; 40%; 50%; 60%; 70%; 80%; 90%; 95%; 98%; 99%; 100%, or more) of the ability of the full-length polypeptide to enantioselectively hydrolyse a IE of interest. Fragments of interest can be made by either of recombinant, synthetic, or proteolytic digestive methods and tested for their ability to enantioselectively hydrolyse a IE.

As used herein, “operably linked”, as applied to a coding sequence of interest, means incorporated into a genetic construct so that an expression control sequence in the genetic construct effectively controls expression of the coding sequence.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., IE and ID preparations substantially enriched for one optical enantiomer, will be apparent from the following description, from the drawings and from the claims.

### DESCRIPTION OF DRAWINGS

FIG. 1 is a depiction of the chemical structures of representative epoxides of general formula (I), (II), (III) and (VII) used to demonstrate the use of YEIH for the synthesis of IE and ID.

FIG. 2 is a GC (gas chromatography)/MS (mass spectrometry) profile of the reaction mixture of the hydrolysis of linoleic acid bisepoxide by the YEIH of Rhodotorula glutinis UOFS Y-0123.

FIG. 3 is a depiction of the chemical structures of products formed during the hydrolysis of linoleic acid bisepoxide by the YEIH of Rhodotorula glutinis UOFS Y-0123).

FIG. 4 is a depiction of the chemical structure of the compound corresponding to the major MS peak generated during hydrolysis of linoleic acid bisepeoxide by a YEIH. The compound is 10,13-dihydroxy-9 (12)-oxyoctadecanoate.

FIG. 5 is a restriction map of the pYLHmA (pINA1291) expression vector used to generate YL-HmA transformants (Yarrowia Lipolytica expression vector, with Hp4d promoter, Multi-copy integration selection, A=no secretion signal). The positions of the hp4d promoter and LIP2 terminator and of unique restriction sites available for the insertion of coding sequences are indicated.

FIG. 6 is a restriction map of the pYL-TsA (pINA3313) integrative vector used to generate YL-Tsa transformants. Restriction enzyme sites include the unique sites available for insertion of sequences under control of the TEF promoter and LIP2 terminator.

FIGS. 7A and 7B are line graphs showing the enantioselective hydrolysis of trans-1-phenylpropene oxide by Trichosporon mucoides NCYC 3206. FIG. 7A shows the change in concentrations of the IE and ID enantiomers with time. The ID forms in an enantioconvergent fashion. Both IE enantiomers are hydrolysed (at different rates) but only one of the ID enantiomers is formed during the first 58% conversion of the epoxide. In this figure and FIGS. 8-17 below, the biocatalyst loadings are indicated in brackets beside the strain names in each graph. The percentage biocatalyst loading refers to a percentage wet weight of yeast cells in the aqueous fraction of the reaction matrix. The value of the percentage wet weight of biocatalyst is approximately five-fold the value of the equivalent dry weight of biocatalyst; and the substrate used in the biotransformation is indicated at the top of the figure and its starting concentration is shown in brackets. FIG. 7B shows the enantiomeric excess of the (1R,2R)-IE and the (1S,2R) ID at different conversions.

FIGS. 8A and 8B show the enantioselective hydrolysis of trans-1-phenylpropene oxide by recombinant host strains transformed with the YEIH of Rhodosporidium toruloides strains (#46=UOFS Y-0471 and #1=NCYC 3181).

FIGS. 9A and 9B show the asymmetrisation of cis-2,3-epoxybutane to (R,R)-2,3-butanediol by Rhodotorula glutinis NCYC 3203 (FIG. 9A) and Rhodotorula araucariae NCYC 3183 (FIG. 9B). The left y-axis (and lines with shaded circle and triangle data points) in each graph show the changes in concentrations of the optically active ID products at the time points indicated on the x-axis and the right y-axis (and lines with shaded diamond-shaped data points) in each graph show the enantiomeric excesses (“ee”) of the formed optically active ID at the time points indicated on the x-axis.

FIGS. 10A and 10B show the asymmetrisation of cis-2,3-epoxybutane to (R,R)-2,3-butanediol by recombinant host strains YL-25 HmA and YL-46 HmA expressing the YEIH from Rhodotorula araucariae (#25=NCYC 3183) (FIG. 10A) and Rhodoporidium toruloides (#46=UOFS Y-0471) (FIG. 10B). The left y-axis (and lines with shaded circle, square and triangle data points) in each graph show the changes in concentrations of the optically active ID products at the time points indicated on the x-axis and the right y-axis (and lines with shaded diamond-shaped data points) in each graph show the enantiomeric excesses (“ee”) of the formed optically active ID at the time points indicated on the x-axis.

FIG. 11 is a line graph showing the hydrolysis of indene oxide by Rhodotorula glutinis NCYC 3186 to produce optically active (1R,2S)-indene oxide. The lines with shaded circle and shaded triangle data points show the changes in concentrations of the IE at the time points indicated on the x-axis.

FIGS. 12A-C are line graphs showing the hydrolysis of indene oxide by YL-TsA and YL-HmA transformants expressing the epoxide hydrolases of #1 (Rhodosporidium toruloides NCYC 3181) (FIG. 12A), #23 (Rhodotorula mucilaginosa UOFS Y-0198) (FIG. 12B) and #692 (Rhodosporidium paludigenum NCYC 3179) (FIG. 12C). For the reaction with YL-692 HmA, double the amount of indene oxide (200 mM) was used compared to the other reactions.

FIG. 13 is a line graph that shows the hydrolysis of indene oxide by YL-692 HmA at 2.5% wet weight cell loading (equal to 0.5% dry weight catalyst) and 200 mM indene oxide.

FIG. 14 is a line graph that shows shows the hydrolysis of indene oxide at 2M substrate loading and 15% wet weight cell loading of the catalyst in the aqueous phase.

FIG. 15 is a line graph that shows the correlation of the enantiomeric excesses at different conversions during the hydrolysis of indene oxide at 200 mM and 2M indene oxide.

FIG. 16 is a line graph showing the hydrolysis of (+)-limonene-1,2-epoxide by a wild type yeast strain, Pichia haplophila NCYC 3176.

FIG. 17 is a line graph showing the hydrolysis of 6,7-epoxygeranyl-1-ol by a Yarrowia lipolytica transformant expressing the epoxide hydrolase from Rhodosporidium toruloides NCYC 3181 (YL-1 HmA).

FIG. 18 is a depiction of the amino acid sequence (SEQ ID NO:1) of a YEIH encoded by cDNA derived from a Rhodosporidium toruloides strain (assigned accession no. NCYC 3181 (#1)).

FIG. 19 is a depiction of the amino acid sequence (SEQ ID NO:2) of a YEIH encoded by cDNA derived from a Rhodosporidium toruloides strain (assigned accession no. UOFS Y-0471 (#46)).

FIG. 20 is a depiction of the amino acid sequence (SEQ ID NO:3) of a YEIH encoded by cDNA derived from a Rhodosporidium paludigenum strain (assigned accession no. NCYC 3179 (#692)).

FIG. 21 is a depiction of the amino acid sequence (SEQ ID NO:4) of a YEIH encoded by cDNA derived from a Rhodosporidium araucariae strain (assigned accession no. NCYC 3183 (#25)).

FIG. 22 is a depiction of the amino acid sequence (SEQ ID NO:5) of a YEIH encoded by cDNA derived from a Cryptococcus curvatus strain (assigned accession no. NCYC 3158 (Car 054)).

FIG. 23 is a depiction of the amino acid sequence (SEQ ID NO:6) of a YEIH encoded by cDNA derived from a Filobasidiella (Cryptococcus) neoformans var neoformans (strain #777) (assigned Genbank accession no. XM_{—}568708).

FIG. 24 is a depiction of the amino acid sequence (SEQ ID NO:7) of a YEIH encoded by cDNA derived from a Rhodotorula mucilaginosa strain (assigned accession no. NCYC 3190 (#23)).

FIG. 25 is a depiction of the nucleotide sequence (SEQ ID NO:8) of a YEIH encoding cDNA derived from a Rhodosporidium toruloides strain (assigned accession no. NCYC 3181 (#1)).

FIG. 26 is a depiction of the nucleotide sequence (SEQ ID NO:9) of a YEIH encoding cDNA derived from a Rhodosporidium toruloides strain (assigned accession no. UOFS Y-0471 (#46)).

FIG. 27 is a depiction of the nucleotide sequence (SEQ ID NO:10) of a YEIH encoding cDNA derived from a Rhodosporidium paludigenum strain (assigned accession no. NCYC 3179 (#692)).

FIG. 28 is a depiction of the nucleotide sequence (SEQ ID NO:11) of a YEIH encoding cDNA derived from a Rhodosporidium araucariae strain (assigned accession no. NCYC 3183 (#25)).

FIG. 29 is a depiction of the nucleotide sequence (SEQ ID NO:12) of a YEIH encoding cDNA derived from a Cryptococcus curvatus strain (assigned accession no. NCYC 3158 (Car 054)).

FIG. 30 is a depiction of the nucleotide sequence (SEQ ID NO:13) of a YEIH encoding cDNA derived from a Filobasidiella neoformans var neoformans (strain #777) (assigned Genbank accession no. XM_{—}568708).

FIG. 31 is a depiction of the nucleotide sequence (SEQ ID NO:14) of a YEIH encoding cDNA derived from a Rhodotorula mucilaginosa strain (assigned accession no. NCYC 3190 (#23)).

FIG. 32 is a table showing the homology at the amino acid level of yeast epoxide hydrolases that are enantioselective for IE.

FIG. 33 is a table showing the homology at the nucleotide level of yeast epoxide hydrolases that are enantioselective for IE.

FIGS. 34A-F are a depiction of amino acid sequence alignments of the YEIH of SEQ ID NO: 1-7. Regions of 50% or greater identity are boxed and identical amino acids are shaded in black. Gaps in individual sequences are represented by “.” Conserved sequence motifs and regions surrounding the catalytic triad are also indicated. The nucleophile, acid and base of the catalytic triad are indicated by N, A and B, respectively. HGXP represents the region of the oxy-anion hole of the enzyme. sxNxss represents the genetic motif found in a/β-hydrolase fold enzymes.

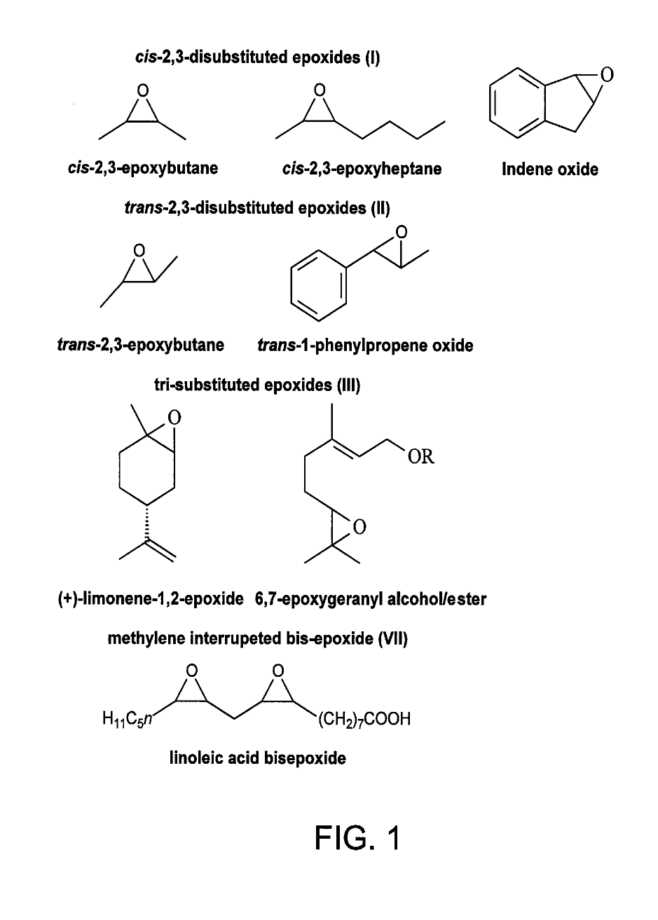

### DETAILED DESCRIPTION

Various aspects of the invention are described below.

### Nucleic Acid Molecules

The YEIH nucleic acid molecules of the invention, or those useful for the invention, can be cDNA, genomic DNA, synthetic DNA, or RNA, and can be double-stranded or single-stranded (i.e., either a sense or an antisense strand). Segments of these molecules are also considered within the scope of the invention, and can be produced by, for example, the polymerase chain reaction (PCR) or generated by treatment with one or more restriction endonucleases. A ribonucleic acid (RNA) molecule can be produced by in vitro transcription. Preferably, the nucleic acid molecules encode polypeptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide (for example, the polypeptides with SEQ ID NOS:1-7). In addition, these nucleic acid molecules are not limited to coding sequences, e.g., they can include some or all of the non-coding sequences that lie upstream or downstream from a coding sequence.

Nucleic acid molecules of the invention, or those useful for the invention, can be synthesized (for example, by phosphoramidite-based synthesis) or obtained from a biological cell, such as the cell of a eukaryote (e.g., a mammal such as human or a mouse or a yeast such as any of the genera, species, and strains of yeast disclosed herein) or a prokarote (e.g., a bacterium such as Escherichia coli). The nucleic acids can be those of a yeast such as any of the genera, species, and strains of yeast disclosed herein. Combinations or modifications of the nucleotides within these types of nucleic acids are also encompassed by the invention.

In addition, the isolated nucleic acid molecules of the invention include segments that are not found as such in the natural state. Thus, the invention encompasses recombinant nucleic acid molecules (for example, isolated nucleic acid molecules encoding the polypeptides of SEQ ID NOs: 1-7) incorporated into a vector (for example, a plasmid or viral vector) or into the genome of a heterologous cell (or the genome of a homologous cell, at a position other than the natural chromosomal location). Recombinant nucleic acid molecules and uses thereof are discussed further below.

Techniques associated with detection or regulation of genes are well-known to skilled artisans. Such techniques can be used, for example, to test for expression of a YEIH gene in a test cell (e.g. a yeast cell) of interest.

A YEIH family gene or protein can be identified based on its similarity to the relevant YEIH gene or protein, respectively. For example, the identification can be based on sequence identity. The invention features isolated nucleic acid molecules which are, or are at least 50% (e.g., at least: 55%; 60%; 65%; 75%; 85%; 95%; 98%; or 99%) identical to: (a) a nucleic acid molecule that encodes the polypeptide of SEQ ID NOs: 1-7; (b) the nucleotide sequence of SEQ ID NOs: 8-14; (c) a nucleic acid molecule which includes a segment of at least 15 (e.g., at least: 20; 25; 30; 35; 40; 50; 60; 80; 100; 125; 150; 175; 200; 250; 300; 350; 400; 500; 600; 700; 800; 900; 1,000; 1,100; 1,181; 1,182; 1,183; 1,184; 1,200; 1,201; 1,202; 1,203; 1,204; 1,205; 1,220; 1,225; 1,228; 1,230; 1,231; 1,232; 1,233; 1,234; or 1,235) nucleotides of SEQ ID NOs: 8-14; (d) a nucleic acid molecule encoding any of the polypeptides or fragments thereof disclosed below; and (e) the complement of any of the above nucleic acid molecules. The complements of the above molecules can be full-length complements or segment complements containing a segment of at least 15 (e.g., at least: 20; 25; 30; 35; 40; 50; 60; 80; 100; 125; 150; 175; 200; 250; 300; 350; 400; 500; 600; 700; 800; 900; 1,000; 1,100; 1,181; 1,182; 1,183; 1,184; 1,200; 1,201; 1,202; 1,203; 1,204; 1,205; 1,220; 1,225; 1,228; 1,230; 1,231; 1,232; 1,233; 1,234; or 1,235) consecutive nucleotides complementary to any of the above nucleic acid molecules. Identity can be over the full-length of SEQ ID NOs: 8-14 or over one or more contiguous or non-contiguous segments.

The determination of percent identity between two sequences is accomplished using the mathematical algorithm of Karlin and Altschul, Proc. Natl. Acad. Sci. USA 90, 5873-5877, 1993. Such an algorithm is incorporated into the BLASTN and BLASTP programs of Altschul et al. (1990) J. Mol. Biol. 215, 403-410. BLAST nucleotide searches are performed with the BLASTN program, score=100, wordlength=12, to obtain nucleotide sequences homologous to YEIH-encoding nucleic acids. BLAST protein searches are performed with the BLASTP program, score=50, wordlength=3, to obtain amino acid sequences homologous to the YEIH polypeptide. To obtain gapped alignments for comparative purposes, Gapped BLAST is utilized as described in Altschul et al. (1997) Nucleic Acids Res. 25, 3389-3402. When utilizing BLAST and Gapped BLAST programs, the default parameters of the respective programs (e.g., XBLAST and NBLAST) are used.

Hybridization can also be used as a measure of homology between two nucleic acid sequences. A YEIH-encoding nucleic acid sequence, or a portion thereof, can be used as a hybridization probe according to standard hybridization techniques. The hybridization of a YEIH probe to DNA or RNA from a test source (e.g., a mammalian cell) is an indication of the presence of YEIH DNA or RNA in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2× sodium chloride/sodium citrate (SSC) at 30° C., followed by a wash in 1×SSC, 0.1% SDS at 50° C. Highly stringent conditions are defined as equivalent to hybridization in 6× sodium chloride/sodium citrate (SSC) at 45° C., followed by a wash in 0.2×SSC, 0.1% SDS at 65° C.

The invention also encompasses: (a) vectors (see below) that contain any of the foregoing YEIH coding sequences (including coding sequence segments) and/or their complements (that is, “antisense” sequences); (b) expression vectors that contain any of the foregoing YEIH coding sequences (including coding sequence segments) operably linked to one or more transcriptional and/or translational regulatory elements (TRE; examples of which are given below) necessary to direct expression of the coding sequences; (c) expression vectors encoding, in addition to a YEIH polypeptide (or a fragment thereof), a sequence unrelated to YEIH, such as a reporter, a marker, or a signal peptide fused to YEIH; and (d) genetically engineered host cells (see below) that contain any of the foregoing expression vectors and thereby express the nucleic acid molecules of the invention.

Recombinant nucleic acid molecules can contain a sequence encoding a YEIH polypeptide or a YEIH polypeptide having an heterologous signal sequence. The full length YEIH polypeptide, or a fragment thereof, can be fused to such heterologous signal sequences or to additional polypeptides, as described below. Similarly, the nucleic acid molecules of the invention can encode the mature form of a YEIH or a form that includes an exogenous polypeptide that facilitates secretion.

The TRE referred to above and further described below include, but are not limited to, inducible and non-inducible promoters, enhancers, operators and other elements that are known to those skilled in the art and that drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast a mating factors. Other useful TRE are listed in the examples below.

Similarly, the nucleic acid can form part of a hybrid gene encoding additional polypeptide sequences, for example, a sequence that functions as a marker or reporter. Examples of marker and reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding β-galactosidase), and xanthine guanine phosphoribosyltransferase (XGPRT), and green, blue, or yellow fluorescent protein. As with many of the standard procedures associated with the practice of the invention, skilled artisans will be aware of additional useful reagents, for example, additional sequences that can serve the function of a marker or reporter. Generally, the hybrid polypeptide will include a first portion and a second portion; the first portion being YEIH polypeptide (including of YEIH fragments described below) and the second portion being, for example, the reporter described above or an Ig heavy chain constant region or part of an Ig heavy chain constant region, e.g., the CH2 and CH3 domains of IgG2a heavy chain. Other hybrids could include an antigenic tag or a poly-Histidine (e.g., hexahistidine) tag to facilitate purification.

The expression systems that can be used for purposes of the invention include, but are not limited to, microorganisms such as yeasts (e.g., any of the genera, species or strains listed herein) or bacteria (for example, E. coli and B. subtilis) transformed with recombinant bacteriophage DNA, plasmid DNA, or cosmid DNA expression vectors containing the nucleic acid molecules of the invention; yeast (for example, Saccharomyces, Kluyveromyces, Hansenula, Pichia, Yarrowia, Arxula, Candida, and other genera, species, and strains listed herein) transformed with recombinant yeast expression vectors containing the nucleic acid molecule of the invention; insect cell systems infected with recombinant virus expression vectors (for example, baculovirus) containing the nucleic acid molecule of the invention; plant cell systems infected with recombinant virus expression vectors (for example, cauliflower mosaic virus (CaMV) or tobacco mosaic virus (TMV)) or transformed with recombinant plasmid expression vectors (for example, Ti plasmid) containing a YEIH polypeptide-encoding nucleotide sequence; or mammalian cell systems (for example, COS, CHO, BHK, 293, VERO, HeLa, MDCK, WI38, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter). Also useful as host cells are primary or secondary cells obtained directly from a mammal and transfected with a plasmid vector or infected with a viral vector.

The invention includes wild-type and recombinant cells including, but not limited to, yeast cells (e.g., any of those disclosed herein) containing any of the above YEIH polypeptide-encoding sequences, nucleic acid molecules, and genetic constructs. Other cells that can be used as host cells are listed herein. The cells are preferably isolated cells. As used herein, the term “isolated” as applied to a microorganism (e.g., a yeast cell) refers to a microorganism which either has no naturally-occurring counterpart (e.g., a recombinant microorganism such as a recombinant yeast) or has been extracted and/or purified from an environment in which it naturally occurs. Thus, an “isolated microorganism” does not include one residing in an environment in which it naturally occurs, for example, in the air, outer space, the ground, oceans, lakes, rivers, and streams and the like, ground at the bottom of oceans, lakes, rivers, and streams and the like, snow, ice on top of the ground or in/on oceans lakes, rivers, and streams and the like, man-made structures (e.g., buildings), or in natural hosts (e.g., plant, animal or microbial hosts) of the microorganism, unless the microorganism (or a progenitor of the microorganism) was previously extracted and or purified from an environment in which it naturally occurs and subsequently returned to such an environment or any other environment in which it can survive. An example of an isolated microorganism is one in a substantially pure culture of the microorganism.

Moreover the invention provides a substantially pure culture of microorganisms (e.g., microbial cells such as yeast cells), a substantial number (i.e., at least 40% (e.g., at least: 50%; 60%; 70%; 80%; 85%; 90%; 95%: 97%; 98%; 99%; 99.5%; or even 100%) of which contain an exogenous nucleic acid encoding an epoxide hydrolase. As used herein, a “substantially pure culture” of a microorganism is a culture of that microorganism in which less than about 40% (i.e., less than about: 35%; 30%; 25%; 20%; 15%; 10%; 5%; 2%; 1%; 0.5%; 0.25%; 0.1%; 0.01%; 0.001%; 0.0001%; or even less) of the total number of viable microbial cells (bacterial, fungal (including yeast), mycoplasmal, or protozoan cells) in the culture are viable microbial cells other than the microorganism. The term “about” in this context means that the relevant percentage can be 15% percent of the specified percentage above or below the specified percentage. Thus, for example, about 20% can be 17% to 23%. Such a culture of microorganisms includes the microorganisms and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

The microbial cells of the invention can be stored, for example, as frozen cell suspensions, e.g. in buffer containing a cryoprotectant such as glycerol or sucrose, as lyophilized cells. Alternatively, they can be stored, for example, as dried cell preparations obtained, e.g., by fluidised bed drying or spray drying, or any other suitable drying method. Similarly the enzyme preparations can be frozen, lyophilised, or immobilized and stored under appropriate conditions to retain activity.

### Polypeptides and Polypeptide Fragments

The YEIH polypeptides of the invention include all the YEIH polypeptides and fragments of YEIH polypeptides disclosed herein. They can be, for example, the polypeptides with SEQ ID NOs:1-7 and functional fragments of these polypeptides. The polypeptides embraced by the invention also include fusion proteins that contain either full-length or a functional fragment of it fused to unrelated amino acid sequence. The unrelated sequences can be additional functional domains or signal peptides.

The invention features isolated polypeptides which are, or are at least 50% (e.g., at least: 55%; 60%; 65%; 75%; 85%; 95%; 98%; or 99%) identical to the polypeptides with SEQ ID NOs: 1-7. The identity can be over the full-length of the latter polypeptides or over one or more contiguous or non-contiguous segments.

Fragments of YEIH polypeptides are segments of the full-length YEIH polypeptides that are shorter than full-length YEIH polypeptides. Fragments of YEIH polypeptides can contain 5-410 (e.g., 10, 20, 30, 40, 50, 60, 70, 80, 90, 100, 120, 150, 250, 300, 350, 391, 392, 393, 397; 398, 399, 400, 402, 403, 404, 405, 406, 407, 408, or 409) amino acids of SEQ ID NOs:1-7. Fragments of YEIH can be functional fragments or antigenic fragments.

The polypeptides can be any of those described above but with not more 50 (e.g., not more than 50, 45, 40, 35, 30, 25, 20, 17, 14, 12, 10, nine, eight, seven, six, five, four, three, two, or one) conservative substitution(s). Such substitutions can be made by, for example, site-directed mutagenesis or random mutagenesis of appropriate YEIH coding sequences.

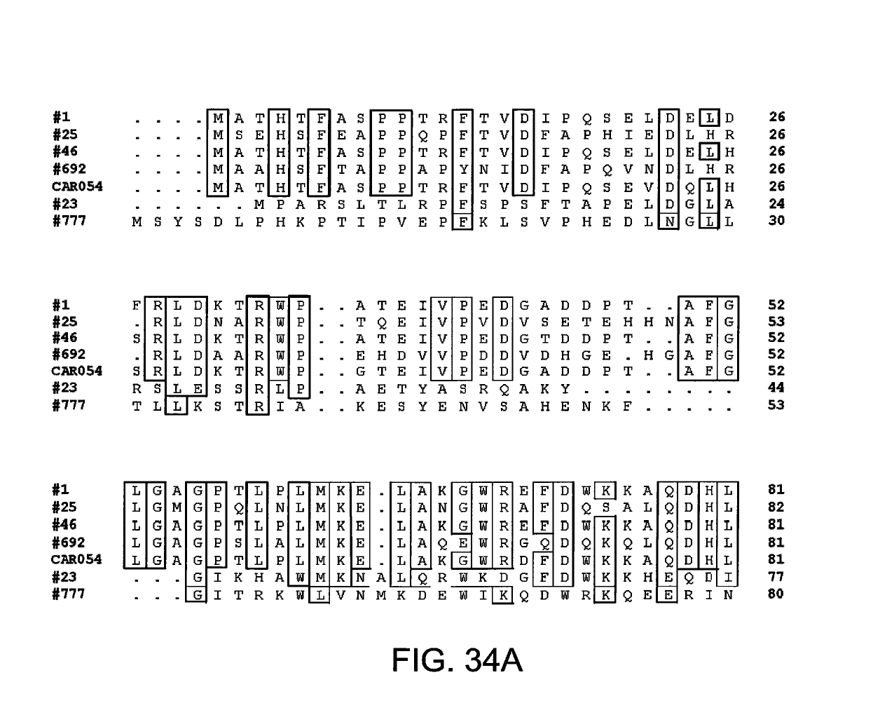

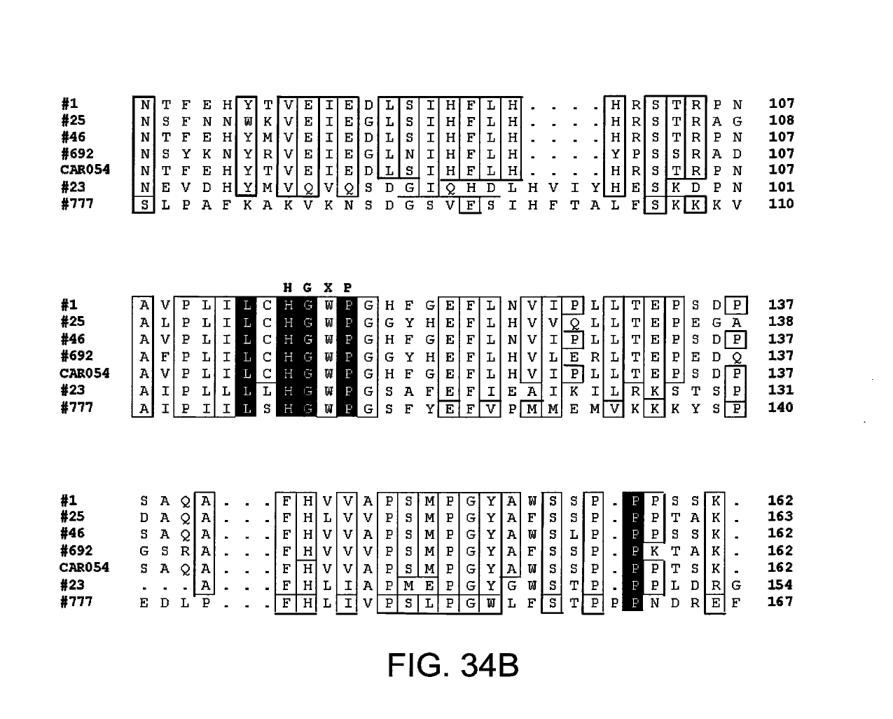

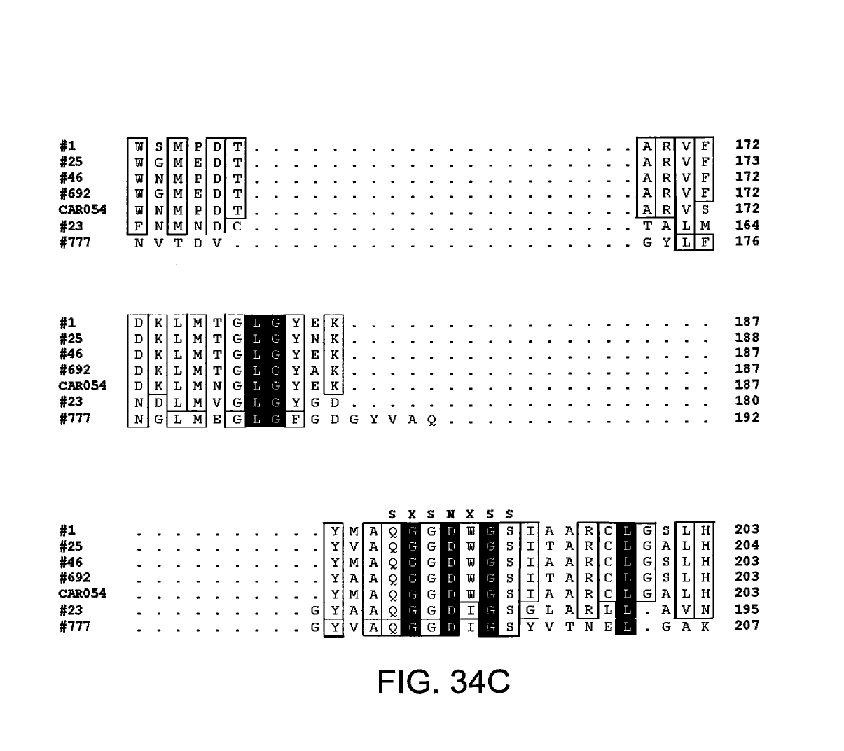

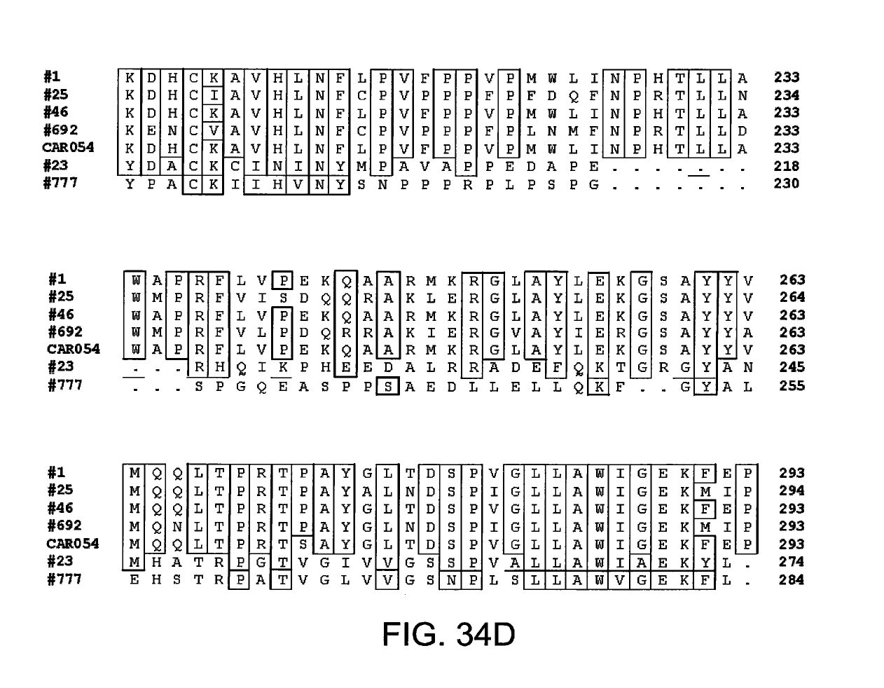

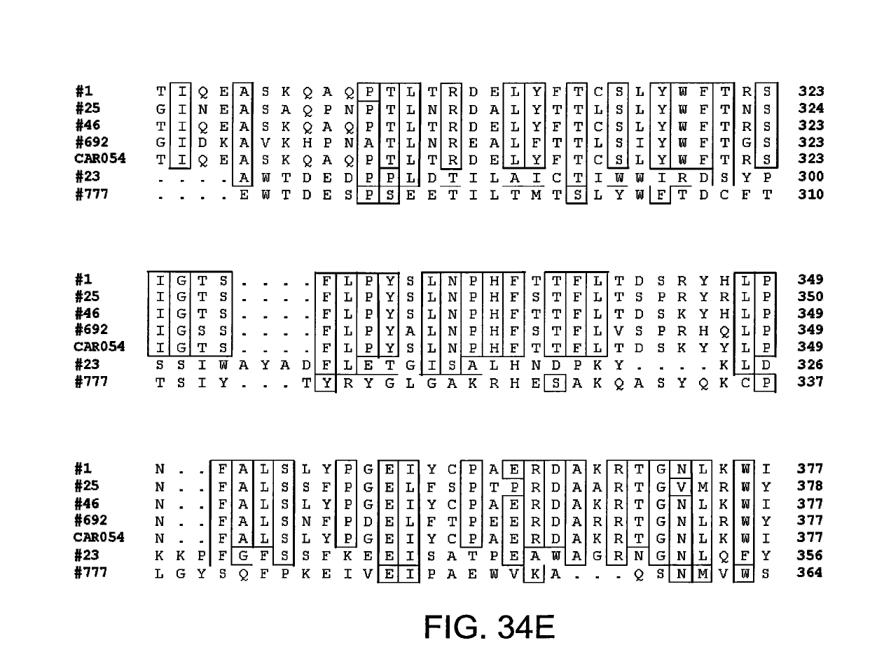

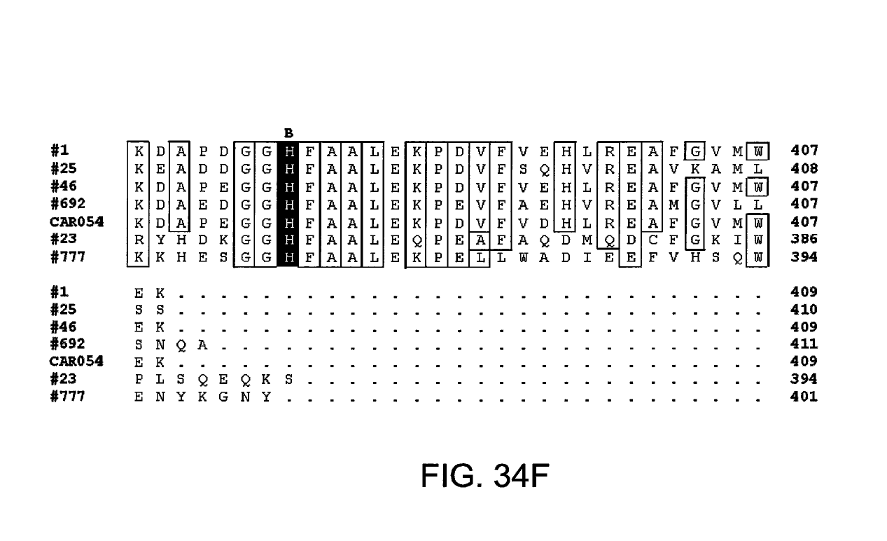

“Functional fragments” of a YEIH polypeptide (and, optionally, any of the above-described YEIH polypeptide variants) have at least 20% (e.g., 30%, 40%, 50%, 60%, 70%, 80%, 90%, 95%, 98%, 99%, 100%, or more) of the ability of the full-length, wild-type YEIH polypeptide to enantioselectively hydrolyse a IE of interest. One of skill in the art will be able to predict YEIH functional fragments using his or her own knowledge and information provided herein, e.g., the amino acid alignments in FIG. 34 showing highly conserved domains as well as residues required for epoxide hydrolase activity in each YEIH.

Fragments of interest can be made either by recombinant, synthetic, or proteolytic digestive methods and tested for their ability to enantioselectively hydrolyse a IE.

Antigenic fragments of the polypeptides of the invention are fragments that can bind to an antibody. Methods of testing whether a fragment of interest can bind to an antibody are known in the art.

The polypeptides can be purified from natural sources (e.g., wild-type or recombinant yeast cells such as any of those described herein). Smaller peptides (e.g., those less than about 100 amino acids in length) can also be conveniently synthesized by standard chemical means. In addition, both polypeptides and peptides can be produced by standard in vitro recombinant DNA techniques and in vivo transgenesis, using nucleotide sequences encoding the appropriate polypeptides or peptides. Methods well-known to those skilled in the art can be used to construct expression vectors containing relevant coding sequences and appropriate transcriptional/translational control signals. See, for example, the techniques described in Sambrook et al., Molecular Cloning: A Laboratory Manual (2nd Ed.) [Cold Spring Harbor Laboratory, N.Y., 1989], and Ausubel et al., Current Protocols in Molecular Biology [Green Publishing Associates and Wiley Interscience, N.Y., 1989].

Polypeptides and fragments of the invention also include those described above, but modified by the addition, at the amino- and/or carboxyl-terminal ends, of a blocking agent to facilitate survival of the relevant polypeptide. This can be useful in those situations in which the peptide termini tend to be degraded by proteases. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptides can be covalently or noncovalently coupled to pharmaceutically acceptable “carrier” proteins prior to administration.

Also of interest are peptidomimetic compounds that are designed based upon the amino acid sequences of the functional peptide fragments. Peptidomimetic compounds are synthetic compounds having a three-dimensional conformation (i.e., a “peptide motif”) that is substantially the same as the three-dimensional conformation of a selected peptide. The peptide motif provides the peptidomimetic compound with the ability to enantioselectively hydrolyse a IE of interest in a manner qualitatively identical to that of the YEIH functional fragment from which the peptidomimetic was derived. Peptidomimetic compounds can have additional characteristics that enhance their therapeutic utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

The invention also provides compositions and preparations containing one or more (e.g., two, three, four, five, six, seven, eight, nine, ten, 12, 15, 20, 25, or more) of the above-described polypeptides, polypeptide variants, and polypeptide fragments. The composition or preparation can be, for example, a crude cell (e.g., yeast cell) extract or culture supernatant, a cell lysate, a crude enzyme preparation, a semi-purified cell extract, or a highly purified enzyme preparation. The compositions and preparations can also contain one or more of a variety of carriers or stabilizers known in the art. Carriers and stabilizers are known in the art and include, for example: buffers, such as phosphate, citrate, and other non-organic acids; antioxidants such as ascorbic acid; low molecular weight (less than 10 residues) polypeptides; proteins such as serum albumin, gelatin or immunoglobulins; hydrophilic polymers such polyvinylpyrrolidone; amino acids such as glycine, glutamine, asparagine, arginine, or lysine; monosaccharides, disaccharides, and other carbohydrates including glucose, mannose, or dextrans; chelating agents such as ethylenediaminetetraacetic acid (EDTA); sugar alcohols such as mannitol, or sorbitol; salt-forming counterions such as sodium; and/or nonionic surfactants such as Tween, and Pluronics.

### Methods of Producing Optically Active Epoxides and Optically Active Vicinal Diols

The invention provides methods for obtaining enantiopure, or substantially enantiopure, optically active IE and ID. Enantiopure optically active IE or ID preparations are preparations containing one enantiomer of the IE or ID and none of the other enantiomer of the IE or ID. “Substantially enantiopure” optically active IE (or ID) preparations are preparations in which the molar amount of the particular enantiomer of the IE (or ID) is at least 55% (e.g., at least: 60%; 70%; 80%; 85%; 90%; 95%; 97%; 98%; 99%; 99.5%; 99.8%; or 99.9%) of the total molar amount of both IE (or ID) enantiomers.

The method involves exposing a IE enantiomeric mixture to a YEIH polypeptide, e.g., by culturing or incubating the mixtures with an isolated YEIH polypeptide or a cell (wild-type or recombinant) that expresses the polypeptide, or with any of the other YEIH-containing preparations disclosed herein. This exposure can have a variety of outcomes that depend on variables such as, without limitation, the YEIH itself, the chemical nature of the IE, and, to a lesser extent, the reaction conditions.

First, the YEIH polypeptide can catalyse the conversion of only one of the two IE enantiomers in the mixture to its corresponding ID enantiomer. Alternatively, it can catalyse the conversion of one of the two IE enantiomers in the mixture to its corresponding ID enantiomer at a much more rapid rate than the other IE enantiomer to its corresponding ID. YEIH with such selective activity are designated “enantioselective” and catalyse the “classical” kinetic resolution of a mixture of IE enantiomers. Such a YEIH polypeptide can catalyse, for example, the conversion of a (2S,3R)-IE (in the IE enantiomeric mixture) to its corresponding (2R,3R)-ID. During such a reaction, the concentration of the selected IE enantiomer decreases, the concentration of the unselected IE remains constant or decreases at a much slower rate (i.e., the concentration of the unselected IE increases relative to the concentration of the selected IE in the mixture); the ID corresponding to the selected IE is produced, and the ID corresponding to the unselected IE is not produced or is produced at a much lower rate than that the ID corresponding to the selected IE. These reactions are of course useful for the enrichment, and hence the production, of a desired IE enantiomer and/or the production of a desired ID enantiomer.

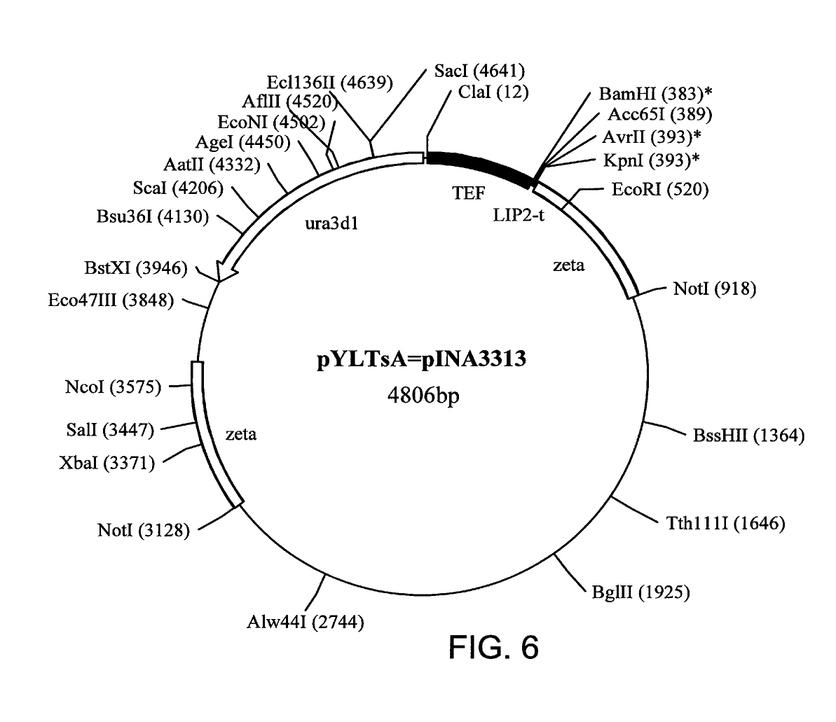

In addition, a YEIH polypeptide can catalyse, under certain defined reaction conditions, the conversion of both enantiomers of a IE to a single ID. Such YEIH hydrolyse each IE enantiomer with opposite regioselectivity (i.e., the attack of water occurs at different carbons of the epoxide ring for each of the IE enantiomers) and are termed “regiospecific” for each IE enantiomer. Such YEIH are said to catalyse the “enantioconvergent” hydrolysis of a mixture of IE enantiomers. The YEIH can also have significant selectivity for one enantiomer of the IE. Where the YEIH has such IE enantiomeric selectivity, the ID enantiomer produced has the same chirality as the IE for which the YEIH polypeptide is selective; for example, if the YEIH polypeptide is selective for a (2S,3R)-IE enantiomer of a cis-2,3-disubstituted epoxide, the ID enantiomer produced from both IE enantiomers will be the (2R,3R)-ID enantiomer; if the YEIH polypeptide is selective for a (2R,3R)-IE enantiomer of a trans-2,3-disubstituted epoxide, the ID enantiomer produced from both IE enantiomers will be the (2S,3R-D) enantiomer. Examples of such reactions include those shown in FIG. 6. During these reactions, there is a decrease in the concentrations of both IE enantiomers (with the concentration of one decreasing faster than the other if the YEIH polypeptide has IE enantiomeric selectivity) and the production of one ID enantiomer. Such reactions are particularly useful for the production of a desired ID enantiomer and, where the YEIH is significantly IE enantioselective, they can also be useful for the enrichment and hence the production of a particular IE enantiomer. The degree of the latter enrichment can be enhanced by, for example, stopping a reaction at a time when the concentration of the selected IE has significantly decreased and that of the unselected IE enantiomer is still relatively high.

In yet other reaction types, the YEIH polypeptide can catalyse, for example: (a) the conversion of one IE enantiomer its corresponding ID enantiomer and the other IE enantiomer to both ID enantiomers; or (b) the conversion of both IE enantiomers to both ID enantiomers. Naturally, such reactions, if the YEIH polypeptides employed have no enantioselectivity, would not be useful for the production of a desired IE enantiomer or a desired ID enantiomer. On the other hand, where the YEIH polypeptide has significant selectivity for one IE enantiomer, the reaction can be used for the production of: (a) the corresponding ID enantiomer; and (b) the unselected IE enantiomer. This can be done, as described above, by, e.g., stopping a reaction at a time, or at times, at which the concentration of the desired IE enantiomer (relative to the total IE concentration) and/or the concentration of the desired ID enantiomer (relative to the total ID concentration) are higher than those of the undesired IE enantiomer and/or the undesired ID enantiomer, respectively. In situations where a YEIH polypeptide catalyses the conversion of one IE enantiomer to both ID enantiomers, advantage can also be taken of the fact that generally the production of one ID enantiomer (e.g., one with the chirality corresponding to that of a selected IE) is favored over the enantiomer.

In view of the above considerations, it will be understood that reactions are enantioselective if the selectivity is: (a) complete (100%), i.e., the reaction results in only one enantiomer of the relevant reaction product; or (b) partial, i.e., the reaction results in a mixture of two enantiomers of the relevant reaction product in which the relative molar amount of one enantiomer is at least 50.1% (e.g., at least: 55%; 60%; 65%; 70%; 80%; 90%; 95%; 97%; 98%; or 99%) of the total molar amount of both enantiomers. The selectivity may also be referred to semiquantitatively as high or low enantioselectivity.

A YEIH polypeptide useful for the invention is one that hydrolyses one enantiomer of a IE, and/or effects the production of one enantiomer of a ID, with less than 80% (e.g., less than: 70%; 60%; 50%; 40%; 30%; 20%; 10%; 5%; 2.5%; 1%; 0.5%; 0.25%; 0.01%, or less), or even none, of the efficiency (as measured by reaction rate) with which it hydrolyses the other IE enantiomer and/or effects the production of the other ID enantiomer, respectively.

Useful concentrations of the IE and conditions of incubation will vary from one YEIH polypeptide to another and from one IE to another. Given the teachings of the working examples contained herein, one skilled in the art will know how to select working conditions for the production of a desired enantiomer of a desired ID and/or IE.

The method can be implemented by, for example, incubating (culturing) the IE enantiomeric mixtures with a wild-type yeast cell or a recombinant cell (yeast or any other host species listed herein) containing a nucleic acid sequence (e.g. a gene, or a recombinant nucleic acid sequence) encoding a YEIH, a crude extract from such cells, a semi-purified preparation of a YEIH polypeptide, or, for example an isolated YEIH polypeptide. As used herein, “incubating and “culturing” include both growing cells and maintaining them in a resting state.

The strain of the yeast cell can be selected from but are not limited to, the following exemplary genera: Arxula, Brettanomyces, Bullera, Bulleromyces, Candida, Cryptococcus, Debaryomyces, Dekkera, Exophiala, Geotrichum, Hormonema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, and Yarrowia.

Yeast strains innately capable of producing a polypeptide which converts or hydrolyses mixtures of cis-2,3-disubstituted IE enantiomers of general formula (I) to optically active (2R,3S)-IE and/or (2R,3R)-ID include, but are not limited to, strains of the exemplary yeast genera and species shown in Table 1.

Yeast strains innately capable of producing a polypeptide which converts or hydrolyses mixtures of trans-2,3-disubstituted IE enantiomers of general formula (II) to optically active (2R,3R)-IE and/or (2R,3S)-ID include, but are not limited to, strains of the exemplary yeast genera and species shown in Table 2.

TABLE 1

| Genus | Species |
|---|---|
| Brettanomyces | B. anomalus |
| | B. bruxellensis |
| | B. sp. UOFS Y-0553 |
| Cryptococcus | C. curvatus |
| | C. macerans |
| | C. podzolicus |
| Debaryomyces | D. hansenii |
| | D. napalensis |
| Dekkera | D. anomala |
| Issatchenkia | I. orientalis |
| Myxozyma | M. melibiosi |
| Rhodosporidium | R. paludigenum |
| | R. lusitaniae |
| | R. toruloides |
| Rhodotorula | R. arauntiaca |
| | R. araucariae |
| | R. glutinis |
| | R. minuta var. minuta |
| Sporidiobolus | S. microsporus |
| | S. salmonicolor |
| Trichosporon | T. delbrueckii |
| | T. pullulans |
| | T. montevideense |
| Yarrowia | Y. lipolytica |

TABLE 2

| Genus | Species |
|---|---|
| Debaryomyces | D. hansenii |
| Exophiala | E. dermatitidis |
| Rhodosporidium e | R. lusitaniae |
| | R. paludigenum |
| Rhodotorula | R. glutinis |
| | R. a minuta |
| | R. mucilaginosa |
| | R. philyla |
| | R. sp. UOFS Y-0560 |
| | R. sp. UOFS Y-2043 |
| Sporidiobolus | S. salmonicolor |
| Trichosporon | T. mucoides |
| | T. pullulans |
| | T. sp. CBS 2488 |
| | T. sp. NCYC 3210 |

Yeast strains innately capable of producing a polypeptide which converts or hydrolyses mixtures of 2,3-tri-substituted IE enantiomers of general formula (III) to optically active IE and/or ID include, but are not limited to, strains of the exemplary yeast genera and species shown in Table 3.

TABLE 3

| Genus | Species |
|---|---|
| Brettanomyces | B. anomalus |
| | B. bruxellensis |
| | B. sp. UOFS Y-0553 |
| Cryptococcus | C. curvatus |
| | C. macerans |
| | C. podzolicus |
| Debaryomyces | D. hansenii |
| | D. napalensis |
| Dekkera | D. anomala |
| Exophiala | E. dermatitidis |
| Issatchenkia | I. orientalis |
| Myxozyma | M. melibiosi |
| Rhodosporidium | R. lusitaniae |
| | R. paludigenum |
| | R. toruloides |
| Rhodotorula | R. araucariae |
| | R. glutinis |
| | R. minuta |
| | R. minuta var. minuta |
| | R. mucilaginosa |
| | R. philyla |
| | R. sp. UOFS Y-0560 |
| | R. sp. UOFS Y-2043 |
| Sporidiobolus | S. microsporus |
| | S. salmonicolor |
| Trichosporon | T. delbrueckii |
| | T. pullulans |
| | T. mucoides |
| | T. pullulans |
| | T. sp. CBS 2488 |
| | T. sp. NCYC 3210 |
| Yarrowia | Y. lipolytica |

Yeast strains innately capable of producing a polypeptide which converts or hydrolyses mixtures of 2,3-tri-substituted IE enantiomers of methylene-interrupted bis-epoxides of general formula (VIII) to optically active epoxides and/or tetraols of general formula (IX) that may undergo ring-closure to form tetrahydrofuran diols of general formula (VIII) include, but are not limited to, the exemplary yeast genera and species shown in Table 4.

TABLE 4

| Genus | Species |
|---|---|
| Bullera | B. dendrophila |
| Candida | C. kruisei |
| Cryptococcus | C. albidus |
| | C. curvatus |
| | C. laurentii |
| | C. podzolicus |
| Exophiala | E. dermatitidis |
| Hormonema | H. sp. |
| Pichia | P. finlandica |
| | P. haplophila |
| Rhodosporidium | R. paludigenum |
| | R. sphaerocarpum |
| | R. toruloides |
| Rhodotorula | R. araucariae |
| | R. aurantiaca |
| | R. glutinis |
| | R. minuta |
| | R. mucilaginosa |
| | R. philyla |
| | R. sp. |
| Sporidiobolus | S. microsporus |
| Sporobolomyces | S. holsaticus |
| Trichosporon | T. beigelii |
| | T. cutaneum var. cutaneum |
| | T. mucoides |
| | T. ovoides |
| | T. sp. |

Yeast species referred to in the tables above and below as “sp.” correspond to strains obtained from a culture collection, the species of which had not at the time of writing been identified.

The yeast strain can be at least one yeast strain selected from the yeast species listed in Table 1, Table 2, Table 3, and Table 4.

Cultivation in bioreactors of wild-type or recombinant yeast strains expressing the polypeptide or fragment thereof having IE enantioselective epoxide hydrolase activity (with the purpose of preparing yeasts stocks or for the enantioselective preparative methods of the invention) can be carried out under conditions that provide useful biomass and/or enzyme titer yields. Cultivation can be by batch, fed-batch or continuous culture methods. Useful cultivation conditions are dependent on the yeast strain used. General procedures for establishing useful culturing conditions of yeasts, fungi and bacteria in bioreactors are known to those skilled in the art. The mixture of epoxides can be added directly to the culture. The concentration of the IE enantiomeric mixture can be at least equal to the solubility of the IE enantiomeric mixture in the aqueous phase of the reaction mixture. The starting amount of epoxide added to the reaction mixture is not critical. The epoxide can be metered out continuously or in batch mode to the reaction mixture. The relative proportions of (R,R)- and (S,S)-epoxide (for trans-2,3-disubstituted epoxides) or ((R,S) and (S,R)-epoxide (for cis-2,3-disubstituted epoxides) or variations thereof for tri-substituted epoxides in the mixture of enantiomers of the epoxide shown by the general formula (I) and (II) and (III) and (VII) is not critical but it is advantageous for commercial purpose to employ a racemic form of the epoxides shown by the general formula (I), (II), (III) and (VII). The epoxide can be added in a racemic form or as a mixture of enantiomers in different ratios.

The amount of the yeast cells, crude yeast cell extract, or partially purified or isolated polypeptide having IE enantioselective activity added to the reaction depends on the kinetic parameters of the specific reaction and the amount of epoxide that is to be hydrolysed. In the case of product inhibition, it can be advantageous to remove the formed vicinal diol from the reaction mixture or to maintain the concentration of the vicinal diol at levels that allow reasonable reaction rates. Techniques used to enhance enzyme and biomass yields include the identification of useful (or optimal) carbon sources, nitrogen sources, cultivation time, dilution rates (in the case of continuous culture) and feed rates, carbon starvation, addition of trace elements and growth factors to the culture medium, and addition of inducers for example substrates or substrate analogs of the epoxide hydrolases during cultivation. In the case of recombinant hosts, the conditions under which the promoters function for transcription of the gene encoding the polypeptide with epoxide hydrolase activity are taken into account. At the end of fermentation (culture), biomass and culture medium can be separated by methods known to one skilled in the art, such as filtration or centrifugation.

The processes are generally performed under Mild conditions. For example, the reaction can be carried out at a pH from 5 to 10, preferably from 6.5 to 9, and most preferably from 7 to 8.5. The temperature for hydrolysis can be from 0 to 70° C., preferably from 0 to 50° C., most preferably from 4 to 40° C. It is also known that lowering of the temperature of the reaction can enhance enantioselectivity of an enzyme.

The reaction mixture can contain mixtures of water with at least one water-miscible solvents (e.g., organic water-miscible solvents). Preferably water-miscible solvents are added to the reaction mixture such that epoxide hydrolase activity remains measurable. Water-miscible solvents are preferably organic solvents and can be, for example, acetone, methanol, ethanol, propanol, isopropanol, acetonitrile, dimethylsulfoxide, N,N-dimethylformamide, N-methylpyrrolidine, and the like.

The reaction mixture can also contain mixtures of water with at least one water-immiscible organic solvent. Examples of water-immiscible solvents that can be used include, for example, toluene, 1,1,2-trichlorotrifluoroethane, methyl tert-butyl ether, methyl isobutyl ketone, dibutyl-o-phtalate, aliphatic alcohols containing 6 to 9 carbon atoms (for example hexanol, octanol), aliphatic hydrocarbons containing 6 to 16 carbon atoms (for example cyclohexane, n-hexane, n-octane, n-decane, n-dodecane, n-tetradecane and n-hexadecane or mixtures of the aforementioned hydrocarbons), and the like. Thus, the reaction mixture can include water with at least one water-immiscible organic solvent selected from the group consisting of toluene, 1,1,2-trichlorotrifluoroethane, methyl tert-butyl ether, methyl isobutyl ketone, dibutyl-o-phtalate, aliphatic alcohols containing 6 to 9 carbon atoms, and aliphatic hydrocarbons containing 6 to 16 carbon.

The reaction mixture can also contain surfactants (for example Tween 80), cyclodextrins or phase-transfer catalysts and the like that can increase, selectively or otherwise, the solubility of the epoxide enantiomers in the reaction mixture.

The reaction mixture can also contain a buffer. Buffers are known in the art and include, for example, phosphate buffers, citrate buffers, TRIS buffers, HEPES buffers, and the like.

The production of the YEIH polypeptides, including functional fragments, can be, for example, as recited above in the section on Polypeptides and Polypeptide Fragments. Thus they can made by production in a natural host cell, production in a recombinant host cell, or synthetic production. Recombinant production can be carried out in host cells of microbial origin. Preferred yeast host cells are selected from, but are not limited to, the genera Saccharomyces, Kluyveromyces, Hansenula, Pichia, Yarrowia, Arxula, and Candida. Preferred bacterial host cells include Escherichia coli, Agrobacterium species, Bacillus species and Streptomyces species. Preferred filamentous fungal host cells are selected from the group consisting of the genera Aspergillus, Trichoderma and Fusarium. The production of the polypeptide can be, e.g., intra- or extra-cellular production and can be by, e.g., secretion into the culture medium.

In the fermentation reactions, the polypeptides (including functional fragments) can be immobilized on a solid support or free in solution. Procedures for immobilization of the yeast or preparation thereof include, but are not limited to, adsorption; covalent attachment; cross-linked enzyme aggregates; cross-linked enzyme crystals; entrapment in hydrogels; and entrapment into reverse micelles.

The progress of the reaction can be monitored by standard procedures known to one skilled in the art, which include, for example, gas chromatography or high-pressure liquid chromatography on columns containing chiral stationary phases. The ID I formed can be removed from the reaction mixture at one or more stages of the reaction.

The reaction can be stopped when one enantiomer of the IE and/or ID is found to be in excess compared to the other enantiomer of the IE and/or ID. Preferably, the reaction is stopped when one enantiomer of the E of general formula (I), (II), (III) and (VII) and/or vicinal diol of general formula (IV), (V), (VI) or (IX) is found to be in an enantiomeric excess of at least 90%. In a more preferred embodiment of the invention, the reaction is stopped when one enantiomer of an IE of general formula (I), (II), (III) and (VII) and/or ID of general formula (IV), (V), (VI), (VIII), or (IX) is found to be in an enantiomeric excess of at least 95%. The reaction can be stopped by the separation (for example centrifugation, membrane filtration, precipitation by solvents, and the like) of the yeast, or preparation thereof, and the reaction mixture or by inactivation (for example by heat treatment or addition of salts and/or organic solvents) of the yeast or polypeptide, or preparation thereof. Thus, the reaction can be stopped by, for example, the separation of the catalytic agent from the reactants and products in the mixture, or by ablation or inhibition of the catalytic activity, by techniques known to one skilled in the art.

The optically active IE and/or ID produced by the reaction can be recovered from the reaction mixture, directly or after removal of the yeast, or preparation thereof. Preferably, the process can include continuously recovering the optically active IE and/or ID produced by the reaction directly from the reaction mixture. Methods of removal of the optically active IE and/or ID produced by the reaction include, for example, extraction with an organic solvent (such as hexane, toluene, diethyl ether, petroleum ether, dichloromethane, chloroform, ethyl acetate and the like), vacuum concentration, crystallisation, distillation, membrane separation, column chromatography and the like.

Thus, the present invention provides an efficient process with economical advantages compared to other chemical and biological methods for the production, in high enantiomeric purity, of optically active IE of the general formula (I), (II), (III) and (VII) and ID of the general formula (IV), (V), (VI), (VIII), or (IX) in the presence of a yeast strain having epoxide hydrolase activity or a polypeptide that is derived from a yeast strain and has such activity.

### YEIH Antibodies

The invention features antibodies that bind to yeast epoxide hydrolase polypeptides or fragments (e.g., antigenic or functional fragments) of such polypeptides. The polypeptides are preferably yeast epoxide polypeptides with enantioselective activity, and in particular those with IE enantioselective activity (i.e., YEIH), e.g., those with SEQ ID NOs: 1, 2, 3, 4, 5, 6 or 7. The antibodies preferably bind specifically to yeast epoxide hydrolase polypeptides, i.e., not to epoxide hydrolase polypeptides of species other than yeast species. More preferably, they can bind specifically to yeast epoxide polypeptides with enantioselective activity, and in particular to YEIH polypeptides, e.g., those with SEQ ID NOs: 1, 2, 3, 4, 5, 6 or 7. They can moreover bind specifically to one or more of polypeptides with SEQ ID NOs: 1, 2, 3, 4, 5, 6, or 7.

Antibodies can be polyclonal or monoclonal antibodies; methods for producing both types of antibody are known in the art. The antibodies can be of any class (e.g., IgM, IgG, IgA, IgD, or IgE). They are preferably IgG antibodies. Moreover, polyclonal antibodies and monoclonal antibodies can be generated in, or generated from, B cells from, animals any number of vertebrate (e.g., mammalian) species, e.g., humans, non-human primates (e.g., monkeys, baboons, or chimpanzees), horses, goats, camels, sheep, pigs, bovine animals (e.g., cows, bulls, or oxen), dogs, cats, rabbits, gerbils, hamsters, guinea pigs, rats, mice, birds (such as chickens or turkeys), or fish.

Recombinant antibodies specific for YEIH polypeptides, such as chimeric monoclonal antibodies composed of portions derived from different species and humanized monoclonal antibodies comprising both human and non-human portions, are also encompassed by the invention. Such chimeric and humanized monoclonal antibodies can be produced by recombinant DNA techniques known in the art, for example, using methods described in Robinson et al., International Patent Publication PCT/US86/02269; Akira et al., European Patent Application 184,187, Taniguchi, European Patent Application 171,496; Morrison et al., European Patent Application 173,494; Neuberger et al., PCT Application WO 86/01533; Cabilly et al., U.S. Pat. No. 4,816,567; Cabilly et al., European Patent Application 125,023; Better et al. (1988) Science 240, 1041-43; Liu et al. (1987) J. Immunol. 139, 3521-26; Sun et al. (1987) PNAS 84, 214-18; Nishimura et al. (1987) Canc. Res. 47, 999-1005; Wood et al. (1985) Nature 314, 446-49; Shaw et al. (1988) J. Natl. Cancer Inst. 80, 1553-59; Morrison, (1985) Science 229, 1202-07; Oi et al. (1986) BioTechniques 4, 214; Winter, U.S. Pat. No. 5,225,539; Jones et al. (1986) Nature 321, 552-25; Veroeyan et al. (1988) Science 239, 1534; and Beidler et al. (1988) J. Immunol. 141, 4053-60.

Also useful for the invention are antibody fragments and derivatives that contain at least the functional portion of the antigen-binding domain of an antibody that binds to a YEIH polypeptide. Antibody fragments that contain the binding domain of the molecule can be generated by known techniques. Such fragments include, but are not limited to: F(ab′)₂ fragments that can be produced by pepsin digestion of antibody molecules; Fab fragments that can be generated by reducing the disulfide bridges of F(ab′)₂ fragments; and Fab fragments that can be generated by treating antibody molecules with papain and a reducing agent. See, e.g., National Institutes of Health, 1 Current Protocols In Immunology, Coligan et al., ed. 2.8, 2.10 (Wiley Interscience, 1991). Antibody fragments also include Fv fragments, i.e., antibody products in which there are few or no constant region amino acid residues. A single chain Fv fragment (scFv) is a single polypeptide chain that includes both the heavy and light chain variable regions of the antibody from which the scFv is derived. Such fragments can be produced, for example, as described in U.S. Pat. No. 4,642,334, which is incorporated herein by reference in its entirety. The antibody can be a “humanized” version of a monoclonal antibody originally generated in a different species.

The above-described antibodies can be used for a variety of purposes including, but not limited to, YEIH polypeptide purification, detection, and quantitative measurement.

The following examples serve to illustrate, not limit, the invention.

### EXAMPLES

### Example I

### Materials and Methods

### Determination of Concentrations and Enantiomeric Excesses

Quantitative determinations of the compounds and determination of enantiomeric excesses were carried out by gas-liquid chromatography (GLC), HPLC (high pressure liquid chromatography) and GC/(gas chromatography)/MS (mass spectrometry). GLC was performed on a Hewlett-Packard 6890 gas chromatograph equipped with FID detector and using H₂ as carrier gas. Determination of the enantiomeric excesses of epoxides and diols was performed by GLC using a fused silica cyclodextrin capillary columns (Supelco) (30 m length, 25 mm ID and 25 μm film thickness). (See Table 5 below).

For analyses of linoleic acid bisepeoxide, linoleic acid tetrahydrofuran diol, and linoleic acid tetraol, the reaction mixtures were extracted with ethyl acetate, methylated with diazomethane and silylated with BSTFA, separated on a DB-5 column, and the eluted peaks were analysed by MS.

TABLE 5

| Compound | Analysis | Column | Temperature |
|---|---|---|---|
| Cis-2,3-epoxybutane | GLC | ?-DEX 110 | 90? C. (isotherm) |
| Cis-2,3-butanediol | GLC | ?-DEX 110 | 90? C. (isotherm) |
| cis-2,3-epoxyheptane | GLC | ChiralDEX | 50? C. (isotherm) |
| | | A-TA | |
| Cis-2,3-heptanediol | GLC | ?-DEX 120 | 120? C. (isotherm) |
| Indene oxide | HPLC | Chiracel | Hexane:ethanol |
| | | OB-H? | (9:1) |
| Indanediol | HPLC | Chiracel | Hexane:ethanol |
| | | OB-H? | (9:1) |
| trans-2,3-butanediol | GLC | ?-DEX 120 | 90? C. (isotherm) |
| trans-phenylpropene | GLC | ?-DEX 120 | 110? C. (isotherm) |
| oxide | | | |
| 1-phenyl-1,2-propane | GLC | ?-DEX 225 | 150? C. (isotherm) |
| diol | | | |
| (+)-limonene-1,2-epoxide | GLC | ?-DEX 120 | 110? C. (isotherm) |
| 6,7-epoxygeranyl alcohol | GLC | ?-DEX 225 | 120? C. (isotherm) |
| Linoleic acid bisepoxide | GC/MS | DB-5 | 150? C.? |
| | | | 5? C./min? |
| | | | 240? C. |
| 11,14-dihydroxy-10 (13)- | GC/MS | DB-5 | 150? C.? |
| oxyoctadecanoate | | | 5? C./min? |
| | | | 240? C. |
| 10,13-dihydroxy-9 (12)- | GC/MS | DB-5 | 150?C.? |
| oxyoctadecanoate | | | 5? C./min? |
| | | | 240? C. |

Concentrations of IE and ID were derived from calibration curves obtained from extractions of the IE and ID from buffer without cells.

### IE and ID Standards

Cis-2,3-epoxyheptane and 6,7-epoxygeranyl alcohol were synthesized from the corresponding alkenes (epoxidation with mCPBA). 2,3-heptanediol was synthesized from 2-heptene (dihydroxylation of alkene). Cis-2,3-epoxybutane and trans-2,3-epoxybutane and 2,3-butanediol were obtained from Fluka (Milan, Italy). (R,R)- and (S,S)-trans-phenylpropene oxide was obtained from Fluka as single enantiomers and mixed for screening reactions. Racemic trans-1-phenylpropene oxide was synthesised from trans-?-methylstyrene (Aldrich) by mCPBA epoxidation. The corresponding diol was obtained by acid hydrolysis of the epoxide. (+)-limonene-1,2-epoxide was purchased from Aldrich. Linoleic acid bisepoxide was synthesized from linoleic acid (epoxidation with mCPBA). Indene oxide was synthesised from indene bromohydrin as described below.

### Synthesis of Racemic Indene Oxide

In a 10 litre glass fermentation reactor having efficient internal cooling coils fitted with baffles and an overhead mechanical stirrer consisting of three impellers attached to the central shaft was placed seven litres (9.65 kg, 172 mol) of a 13M aqueous potassium hydroxide solution.

The solution was cooled to 0 ?C. Indene bromohydrin (472.18 g, 2.22 mol) was added to the solution, and the heterogeneous mixture stirred vigorously (500 rpm) for 18 hours at 0 ?C. The solids were dissolved with dichloromethane (2? 1.25 litres), and left to stir for 30 minutes. After settling for approximately 45 minutes, the upper organic phase of each extraction was isolated, dried (with MgSO₄) and analysed by TLC (thin layer chromatography) for starting material or hydrolysis products. Concentration of the solution in vacuo afforded a brown transparent oil that partially crystallised on cooling. Proton NMR (nuclear magnetic resonance) spectroscopy indicated that the crude product was almost pure indene oxide. Simple distillation (ca. 65 ?C/5 mmHg) afforded indene oxide (276.83 g, 95%) as a clear oil, 99+% pure by GC area %. A similar run with 550.10 g (2.58 mol) of Indene bromohydrin afforded 301.03 g (88%) of the required indene oxide product. The product was stored at 0 ?C in sealed bottled (until used) forming low-melting point crystals.

### Preparation of Frozen Yeast Cells for Screening

Yeasts were grown at 30?C in 1 L shake-flask cultures containing 200 ml yeast extract/malt extract (YM) medium (3% yeast extract, 2% malt extract, 1% peptone w/v) supplemented with 1% glucose (w/v). At late stationary phase (48-72 h), the cells were harvested by centrifugation (10 000 g, 10 min, 4?C), washed with phosphate buffer (50 mM, pH7.5), centrifuged and frozen in phosphate buffer containing glycerol (20%) at −20?C as 20% (w/v) cell suspensions. The cells were stored for several months without significant loss of activity.

### Screening

IE (10 ?l of a 1 M stock solution in EtOH) were added to a final concentration of 20 mM to 500 ?l cell suspension (20% w/v) in phosphate buffer (50 mM, pH 7.5). The reaction mixtures were incubated at 30?C for 2 hours or 5 hours. The reaction mixtures were extracted with EtOAc (300 ?l) and centrifuged. ID formation was evaluated by TLC (silica gel Merck 60 F₂₅₄). Compounds were visualized by spraying with vanillin/conc. H₂SO₄ (5 g/l). Reaction mixtures that showed substantial ID formation were evaluated for asymmetric hydrolysis of the IE by chiral GLC analysis. Some reactions were repeated over longer or shorter times and with more dilute cell suspensions (10% w/v) in order to analyse the reactions at suitable conversions.

General Procedure for the Hydrolysis of 2,3-disubstituted and Tri-Substituted Epoxides

Frozen cells were thawed, washed with phosphate buffer-(50 mM, pH 7.5) and resuspended in buffer. Cell suspensions (10 ml, 20% or 50% w/v) were placed in 20 ml glass bottles with screw caps fitted with septa. The substrate (100 Xl or 250 ?l of a 2M (v/v) stock solution in ethanol) was added to final concentrations of 20 mM or 50 mM. The mixtures were agitated on a shaking water bath at 30?C. The course of the bioconversions of epoxides was followed by withdrawing samples (500 ?l) at appropriate time intervals.

Samples for GLC analysis were extracted with 300 ?l EtOAc. After centrifugation (3000×g, 2 min), the organic layer was dried over anhydrous MgSO₄ and the products analyzed by chiral GLC.

Samples for HPLC analysis were extracted with 300 ?l hexane, centrifuged (12000×g, 10 min), the organic layer dried over anhydrous MgSO₄, and the products analyzed by chiral HPLC analysis.

Samples for GC/MS analysis were extracted with 300 ?l ether. After centrifugation (3000×g, 2 min), the organic layer was dried under a nitrogen stream, redissolved in methanol, and methylated with diazomethane. A portion of the methanol/diazomethane reaction mixture was dried under nitrogen, dissolved in pyridine, and silylated with BSTFA prior to analysis.

### Yeast Strains

Yeast strains with screen numbers denoted “AB” or “Car” or “Alf” or “Poh” were isolated from soil from specialised ecological niches that were selected based on the inventors' belief that selectivity for specific classes of epoxides in microorganisms would be determined by environmental factors such as terpene-rich environments or highly contaminated soil. “AB” and “Alf” strains were isolated from Cape Mountain fynbos, an ecological environment unique to South Africa, “Car” strains were isolated from soil under pine trees, and “Poh” strains from soil contaminated by high concentrations of cyanide. Microorganisms existing in these contaminated soils most likely have alternative respiratory systems. Nearly all these new isolates displayed activity and selectivity for 2,3-disubstituted and tri-substituted epoxides, while only a few of the more than 200 strains from the Yeast Culture Collection that were included in the screening, displayed activity and selectivity for 2,3-disubstituted epoxides. Furthermore, the strains from the culture collection that were found to be able to produce optically active epoxides and/or diols from 2,3-disubstituted epoxides belonged in most cases to the same species as those isolated from soil from the selected environments. All new isolates that produced optically active epoxides or vicinal diols during hydrolysis of 2,3-disubstituted and tri-substituted epoxides were identified by known biochemical characterisation methods, and most of the isolates were also subjected to molecular identification by sequence analyses of the D1/D2 region of the large subunit rDNA. These new isolated were subsequently deposited at the Yeast Culture Collection of the University of the Orange Free State (UOFS) and assigned UOFS numbers.

All the yeast strains mentioned in the examples are kept and maintained at the University of the Orange Free State (UOFS), Department of Microbial, Biochemical and Food Biotechnology, Faculty of Natural and Agricultural Sciences, P.O. Box 339, Bloemfontein 9300, Republic of South Africa. Tel +27 51 401 2396, Fax +27 51 444 3219 and are readily identified by the yeast species and culture collection number as indicated. Representative examples of strains belonging to the different species have been deposited at the NCYC (National Collection of Yeast Cultures Institute of Food Research Norwich Research Park Colney, Norwich NR4 7UA, U.K. Tel: +44-(0)1603-255274 Fax: +44-(0)1603-458-414 Email: ncyc@bbsrc.ac.uk). Samples of these yeast strains will be made available upon request basis on the same and conditions of the Budapest Treaty.

### Example II

### Selection of Wild Type Yeasts for the Production of Optically Active Epoxides and Vicinal Diols from IE

Yeasts were cultivated, harvested and frozen as 20% or 50% (w/v) cell suspensions as described above. The IE was added to a final concentration of 20 mM or 50 mM and the screening was performed as described above. Strains with the highest activities as determined by TLC from diol formation were subjected to chiral GC or HPLC analysis as described above. The enantiomeric excesses of the formed diol after incubation for 1-20 hours of the IE (cis-2,3-epoxybutane; cis-2,3-epoxyheptane, trans-2,3-epoxybutane, linoleic acid bisepoxide, trans-1-phenylpropene oxide and indene oxide) with the different yeast strains are shown as Samples 1-215 in Tables 7-12. Enantiomeric excesses (ee) were determined from the formula: E.e.=[[E-1]−[E-2]}/{[E-1]+[E-2]}×100. [E-1] is the concentration at the appropriate time point of the one enantiomer of the IE or ID and [E-2] is the concentration at the appropriate time point of the other IE or ID enantiomer.

TABLE 7
(Samples 1-47) Enantiomeric excesses (Ee) of formed diol obtained after
incubation of different yeast strains with cis-2,3-epoxybutane
(50 mM, 50% w/v cells, 16 hours).

| | Internal | | | | |
|---|---|---|---|---|---|
| Sample | strain | | Culture | Abs | Ee |
| No. | No. | Strain/species | collection No. | Conf. | (%) |
| 1 | K 01 | Brettanomyces anomalus | NCYC 3149 | R, R | 71.0 |
| 2 | 52 | Brettanomyces bruxellensis | NCYC 3150 | R, R | 19.3 |
| 3 | N 13 | Brettanomyces sp. | NCYC 3151 | R, R | 74.0 |
| 4 | 704 | Candida fabianii | UOFS Y-0963 | R, R | 84.1 |
| 5 | X 19 | Candida kruisii | NCYC 3153 | R, R | 77.7 |
| 6 | Jen 13 | Cryptococcus gastricus | UOFS Y-0477 | R, R | 31.9 |
| 7 | AB 54 | Cryptococcus podzolicus | UOFS Y-1900 | R, R | 27.4 |
| 8 | 110 | Debaryomces hansenii | UOFS Y-0614 | R, R | 59.5 |
| 9 | 108 | Debaryomces hansenii | UOFS Y-0612 | R, R | 86.0 |
| 10 | 93 | Debaryomyces hansenii | NCYC 3169 | R, R | 55.6 |
| 11 | 105 | Debaryomyces hansenii | UOFS Y-0608 | R, R | 93.9 |
| 12 | 111 | Debaryomyces hansenii | UOFS Y-0615 | R, R | 95.8 |
| 13 | TVN 119 | Debaryomyces napalensis | NCYC 3220 | R, R | >98% |
| 14 | 88 | Dekkera anomala | NCYC 3170 | R, R | 17.4 |
| 15 | 227 | Geotricum sp. | UOFS Y-0111 | R, R | 33.4 |
| 16 | TVN 228 | Issatchenkia orientalis | NCYC 3221 | R, R | 60.1 |
| 17 | TVN 229 | Issatchenkia orientalis | NCYC 3222 | R, R | 63.0 |
| 18 | TVN 231 | Issatchenkia orientalis | NCYC 3223 | R, R | 62.5 |
| 19 | 45a | Lipomyces sp. | UOFS Y-2159 | R, R | 77.5 |
| 20 | 41 | Myxozyma melibiosi | NCYC 3172 | R, R | 18.9 |
| 21 | 520* | Pichia finlandica | NCYC 3173 | R, R | >98% |
| 22 | 673 | Pichia haplophila | NCYC 3177 | R, R | 65.7 |
| 23 | 169 | Rhodosporidium lusitaniae | NCYC 3178 | R, R | 31.7 |
| 24 | 40 | Rhodosporidium sphaerocarpum | NCYC 3180 | R, R | 27.4 |
| 25 | Alf 01 | Rhodosporidium toruloides | NCYC 3181 | R, R | 60.0 |
| 26 | 46 | Rhodosporidium toruloides | UOFS Y-0471 | R, R | 45.0 |
| 27 | 2 | Rhodosporidium toruloides | UOFS Y-0518 | R, R | 88.7 |
| 28 | Car 067 | Rhodosporidium toruloides | UOFS Y-2236 | R, R | 49.6 |
| 29 | Car 076 | Rhodosporidium toruloides | UOFS Y-2238 | R, R | 85.3 |
| 30 | Car 131 | Rhodosporidium toruloides | UOFS Y-2252 | R, R | 83.9 |
| 31 | Car 142 | Rhodosporidium toruloides | UOFS Y-2255 | R, R | 43.3 |
| 32 | Car 209 | Rhodosporidium toruloides | UOFS Y-2260 | R, R | 84.0 |
| 33 | 25 | Rhodotorula araucariae | NCYC 3183 | R, R | 81.1 |
| 34 | EP 230 | Rhodotorula aurantiaca | NCYC 3185 | R, R | 41.8 |
| 35 | 50 | Rhodotorula glutinis | NCYC 3186 | R, R | 86.1 |
| 36 | 680 | Rhodotorula glutinis | NCYC 3203 | R, R | 77.9 |
| 37 | 713 | Rhodotorula glutinis | UOFS Y-0489 | R, R | 45.2 |
| 38 | Alf 6 | Rhodotorula glutinis | UOFS Y-0513 | R, R | 95.5 |
| 39 | 185 | Rhodotorula glutinis | UOFS Y-0559 | R, R | 49.5 |
| 40 | 686 | Rhodotorula minuta var. minuta | NCYC 3188 | R, R | 67.2 |
| 41 | Rh 27 | Rhodotorula sp. | NCYC 3224 | R, R | 89.0 |
| 42 | 697 | Rhodotorula sp. minuta/mucilaginosa | UOFS Y-0958 | R, R | 46.7 |
| 43 | 698 | Rhodotorula sp. minuta/mucilaginosa | UOFS Y-0959 | R, R | 50.2 |
| 44 | BV04 | Trichosporon moniliiforme | NCYC 3214 | R, R | 33.6 |
| 45 | 61 | Trichosporon pullulans | NCYC 3209 | R, R | 91.4 |
| 46 | 231 | Trichosporon sp. | NCYC 3210 | R, R | 97.6 |
| 47 | 39 | Wingea robertsiae | NCYC 3228 | R, R | 22.1 |

TABLE 8
(Samples 48-78). E nantiomeric excesses of the remaining
epoxide produced by yeast strains that hydrolyse cis-2,3-
epoxyheptane enantioselectively (50 mM 2,3-epoxyheptane,
50% cells, 1 hour).

| Sample | Screen | | Culture | Ee |
|---|---|---|---|---|
| no | no | Yeast species | collection nr. | (%) |
| 48 | Car 054 | Cryptococcus curvatus | NCYC 3158 | 29.56 |
| 49 | Jen 14 | Cryptococcus macerans | NCYC 3163 | 6.06 |
| 50 | Car 103 | Rhodosporidium toruloides | UOFS Y-2246 | 9.19 |
| 51 | Car 134 | Rhodosporidium toruloides | UOFS Y-2253 | 10.04 |
| 52 | Car 059 | Rhodosporidium toruloides | UOFS Y-2231 | 10.73 |
| 53 | Car 210 | Rhodosporidium toruloides | UOFS Y-2261 | 12.17 |
| 54 | Car 204 | Rhodosporidium toruloides | UOFS Y-2257 | 14.11 |
| 55 | Car 003 | Rhodosporidium toruloides | UOFS Y-2222 | 15.50 |
| 56 | Car 100 | Rhodosporidium toruloides | UOFS Y-2245 | 17.04 |
| 57 | Car 093 | Rhodosporidium toruloides | UOFS Y-2242 | 18.20 |
| 58 | Car 038 | Rhodosporidium toruloides | UOFS Y-2228 | 18.21 |
| 59 | Car 006 | Rhodosporidium toruloides | UOFS Y-2223 | 19.13 |
| 60 | Car 108 | Rhodosporidium toruloides | UOFS Y-2247 | 23.70 |
| 61 | Car 118 | Rhodosporidium toruloides | NCYC 3182 | 24.08 |
| 62 | Car 142 | Rhodosporidium toruloides | UOFS Y-2255 | 26.23 |
| 63 | Car 126 | Rhodosporidium toruloides | UOFS Y-2251 | 26.49 |
| 64 | Car 076 | Rhodosporidium toruloides | UOFS Y-2238 | 27.15 |
| 65 | Car 209 | Rhodosporidium toruloides | UOFS Y-2260 | 27.50 |
| 66 | Car 020 | Rhodosporidium toruloides | UOFS Y-2226 | 28.75 |
| 67 | Car 120 | Rhodosporidium toruloides | UOFS Y-2249 | 29.03 |
| 68 | Car 200 | Rhodosporidium toruloides | UOFS Y-2256 | 30.56 |
| 69 | Car 205A | Rhodosporidium toruloides | UOFS Y-2258 | 32.60 |
| 70 | Car 078 | Rhodosporidium toruloides | UOFS Y-2240 | 34.45 |
| 71 | Car 052 | Rhodosporidium toruloides | UOFS Y-2230 | 36.70 |
| 72 | Car 121 | Rhodosporidium toruloides | UOFS Y-2250 | 38.26 |
| 73 | Car 092 | Rhodosporidium toruloides | UOFS Y-2241 | 38.96 |
| 74 | POH 28 | Rhodosporidium toruloides | NCYC 3215 | 99.50 |
| 75 | I 05 | Rhodosporidium toruloides | UOFS Y-0471 | −4.65 |
| 76 | Jen 37 | Yarrowia lipolytica | UOFS Y-0097 | −7.06 |
| 77 | Jen 36 | Yarrowia lipolytica | UOFS Y-1569 | −6.52 |
| 78 | Jen 46 | Yarrowia lipolytica | UOFS Y-1701 | −3.10 |

Negative ee values denote strains with opposite enantioselectivity.

TABLE 9
(samples 79-97). Yeast strains that hydrolyse trans-2,3-epoxybutane
enantioselectively (reaction conditions: 20 mM IE, 20% cells (w/v), 24 hours). Reaction
mixtures were analysed for the formation of the ID. The concentrations of the ID
enantiomers obtained after 24 hours is given in the table.

| Sample | | | Culture | [meso] | [R, R] | [S, S] |
|---|---|---|---|---|---|---|
| No. | SNO | Strain | Collection no. | (mM) | (mM) | (mM) |
| 79 | K 22 | Debaryomyces hansenii | UOFS Y-0539 | 5.53 | 0.00 | 0.00 |
| 80 | Rh 31 | Exophiala dermatitidis | NCYC 3227 | 8.11 | 0.00 | 0.00 |
| 81 | Rh 25 | Rhodosporidium lusitaniae | UOFS Y-1619 | 10.01 | 0.00 | 0.00 |
| 82 | Rh 38 | Rhodosporidium paludigenum | UOFS Y-0482 | 0.00 | 2.34 | 9.54 |
| 83 | J 03 | Rhodotorula glutinis | UOFS Y-0123 | 14.76 | 0.00 | 0.00 |
| 84 | Rh 10 | Rhodotorula glutinis | UOFS Y-0653 | 7.45 | 0.00 | 0.00 |
| 85 | Rh 09 | Rhodotorula minuta | UOFS Y-0835 | 9.04 | 0.00 | 0.00 |
| 86 | Rh 09 | Rhodotorula minuta | UOFS Y-0835 | 9.47 | 0.00 | 0.00 |
| 87 | Rh 35 | Rhodotorula minuta | UOFS Y-1626 | 10.85 | 0.00 | 0.00 |
| 88 | Rh 05 | Rhodotorula mucilaginosa | UOFS Y-0124 | 7.77 | 0.00 | 0.00 |
| 89 | Rh 23 | Rhodotorula mucilaginosa | UOFS Y-0226 | 4.07 | 0.00 | 0.00 |
| 90 | Rh 12 | Rhodotorula mucilaginosa | UOFS Y-0478 | 5.82 | 0.00 | 0.00 |
| 91 | Rh 43 | Rhodotorula philyla | UOFS Y-0134 | 0.00 | 19.98 | 0.00 |
| 92 | Rh 16 | Rhodotorula sp. | UOFS Y-2043 | 6.48 | 0.00 | 0.00 |
| 93 | Rh 28 | Rhodotorula sp. | UOFS Y-0560 | 6.92 | 0.00 | 0.00 |
| 94 | G 06 | Sporidiobolus salmonicolor | UOFS Y-0856 | 9.25 | 0.00 | 0.00 |
| 95 | K 12 | Trichosporon pullulans | CBS 2538 | 7.50 | 0.00 | 0.00 |
| 96 | K 09 | Trichosporon sp. | CBS 2488 | 0.00 | 8.23 | 0.00 |
| 97 | T 09 | Trichosporon sp. | UOFS Y-0533 | 0.00 | 0.43 | 10.78 |

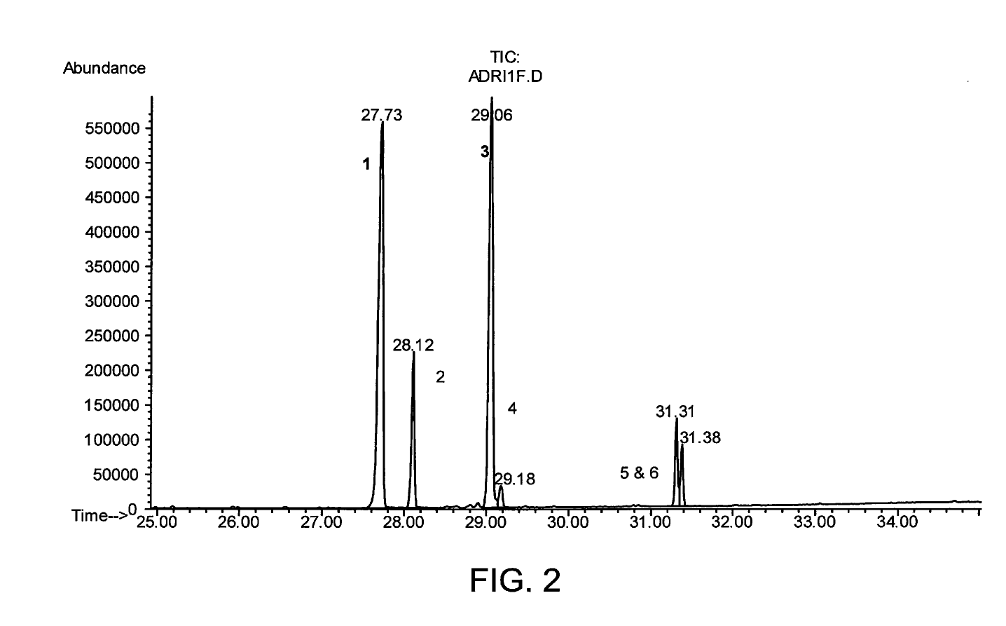

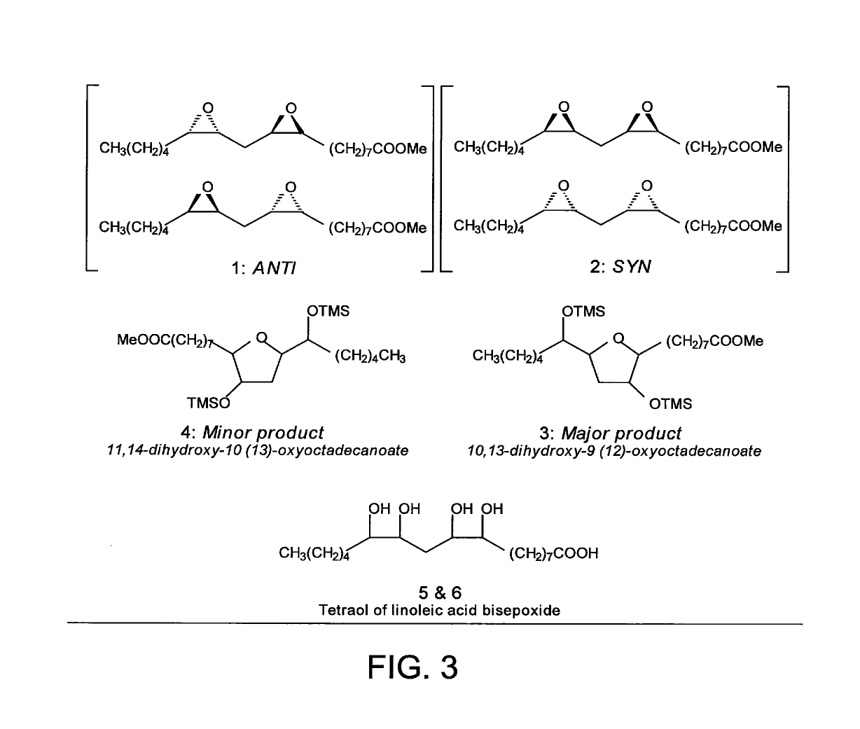

Table 10 lists the yeast strains that are able to hydrolyse linoleic acid bisepoxide with the formation of the THF diol and tetra-ol (FIG. 2); Enantiomer analysis was not done on the THF diols, but the diastereoisomers and positional isomers were identified as shown in FIG. 3.

TABLE 10
(samples 98-182). Hydrolysis of linoleic acid bisepoxide (50 mM) by
selected yeasts (20% w/v) to produce tetrahydrofuran (THF) diols
and tetraols.

| Sample | Screen | | Culture |
|---|---|---|---|
| number | number | Yeast strain | Collection no |
| 98 | 43 | Bullera dendrophila | NCYC 3152 |
| 99 | X 19 | Candida kruisii | NCYC 3153 |
| 100 | Alf 04 | Cryptococcus laurentii | NCYC 3226 |
| 101 | 230 | Cryptococcus albidus | NCYC 3156 |
| 102 | Car 014 | Cryptococcus curvatus | UOFS Y-2225 |
| 103 | AB 24 | Cryptococcus laurentii | NCYC 3161 |
| 104 | AB 25 | Cryptococcus laurentii | UOFS Y-1884 |
| 105 | AB 26 | Cryptococcus laurentii | UOFS Y-1885 |
| 106 | AB 32 | Cryptococcus laurentii | UOFS Y-1887 |
| 107 | AB 33 | Cryptococcus laurentii | UOFS Y-1888 |
| 108 | AB 53 | Cryptococcus podzolicus | NCYC 3165 |
| 109 | 173 | Exophialadermatitidis | NCYC 3227 |
| 110 | Rh39 | Hormonema sp. | NCYC 3171 |
| 111 | 520 | Pichia finlandica | NCYC 3173 |
| 112 | 673 | Pichia haplophila | NCYC 3177 |
| 113 | 692 | Rhodosporidium paludigenum | NCYC 3179 |
| 114 | 40 | Rhodosporidium sphaerocarpum | NCYC 3180 |
| 115 | 46 | Rhodosporidium toruloides | UOFS Y-0471 |
| 116 | Alf 01 | Rhodosporidium toruloides | NCYC 3181 |
| 117 | Alf 02 | Rhodosporidium toruloides | UOFS Y-0518 |
| 118 | Car 003 | Rhodosporidium toruloides | UOFS Y-2222 |
| 119 | Car 006 | Rhodosporidium toruloides | UOFS Y-2223 |
| 120 | Car 038 | Rhodosporidium toruloides | UOFS Y-2228 |
| 121 | Car 052 | Rhodosporidium toruloides | UOFS Y-2230 |
| 122 | Car 059 | Rhodosporidium toruloides | UOFS Y-2231 |
| 123 | Car 067 | Rhodosporidium toruloides | UOFS Y-2236 |
| 124 | Car 070 | Rhodosporidium toruloides | UOFS Y-2237 |
| 125 | Car 076 | Rhodosporidium toruloides | UOFS Y-2238 |
| 126 | Car 077 | Rhodosporidium toruloides | UOFS Y-2239 |
| 127 | Car 078 | Rhodosporidium toruloides | UOFS Y-2240 |
| 128 | Car 092 | Rhodosporidium toruloides | UOFS Y-2241 |
| 129 | Car 093 | Rhodosporidium toruloides | UOFS Y-2242 |
| 130 | Car 094 | Rhodosporidium toruloides | UOFS Y-2243 |
| 131 | Car 100 | Rhodosporidium toruloides | UOFS Y-2245 |
| 132 | Car 103 | Rhodosporidium toruloides | UOFS Y-2246 |
| 133 | Car 108 | Rhodosporidium toruloides | UOFS Y-2247 |
| 134 | Car 118 | Rhodosporidium toruloides | NCYC 3182 |
| 135 | Car 120 | Rhodosporidium toruloides | UOFS Y-2249 |
| 136 | Car 121 | Rhodosporidium toruloides | UOFS Y-2250 |
| 137 | Car 126 | Rhodosporidium toruloides | UOFS Y-2251 |
| 138 | Car 131 | Rhodosporidium toruloides | UOFS Y-2252 |
| 139 | Car 134 | Rhodosporidium toruloides | UOFS Y-2253 |
| 140 | Car 142 | Rhodosporidium toruloides | UOFS Y-2255 |
| 141 | Car 204 | Rhodosporidium toruloides | UOFS Y-2257 |
| 142 | Car 205A | Rhodosporidium toruloides | UOFS Y-2258 |
| 143 | Car 209 | Rhodosporidium toruloides | UOFS Y-2260 |
| 144 | Car 210 | Rhodosporidium toruloides | UOFS Y-2261 |
| 145 | C 09 | Rhodotorula araucariae | NCYC 3183 |
| 146 | EP 230 | Rhodotorula aurantiaca | NCYC 3185 |
| 147 | 50 | Rhodotorula glutinis | NCYC 3186 |
| 148 | 185 | Rhodotorula glutinis | UOFS Y-0559 |
| 149 | 680 | Rhodotorula glutinis | NCYC 3203 |
| 150 | 681 | Rhodotorula glutinis | UOFS Y-0653 |
| 151 | 713 | Rhodotorula glutinis | UOFS Y-0489 |
| 152 | Alf 06 | Rhodotorula glutinis | UOFS Y-0513 |
| 153 | Car 022 | Rhodotorula glutinis | UOFS Y-2227 |
| 154 | Car 060 | Rhodotorula glutinis | UOFS Y-2232 |
| 155 | Car 061 | Rhodotorula glutinis | UOFS Y-2233 |
| 156 | Car 062 | Rhodotorula glutinis | UOFS Y-2234 |
| 157 | Car 066 | Rhodotorula glutinis | UOFS Y-2235 |
| 158 | Car 075 | Rhodotorula glutinis | UOFS Y-2265 |
| 159 | Rh24 | Rhodotorula glutinis | UOFS Y-0519 |
| 160 | 714 | Rhodotorula minuta | NCYC 3187 |
| 161 | Rh46 | Rhodotorula minuta | UOFS Y-0126 |
| 162 | 686 | Rhodotorula minuta var. minuta | NCYC 3188 |
| 163 | 712 | Rhodotorula minuta var. minuta | UOFS Y-0835 |
| 164 | 23 | Rhodotorula mucilaginosa | NCYC 3190 |
| 165 | 174 | Rhodotorula philyla | NCYC 3191 |
| 166 | 24 | Rhodotorula sp. | UOFS Y-2042 |
| 167 | 37 | Rhodotorula sp. | UOFS Y-0448 |
| 168 | 165 | Rhodotorula sp. | NCYC 3193 |
| 169 | 172 | Rhodotorula sp. | NCYC 3192 |
| 170 | 158 | Rhodotorula sp. minuta/ | NCYC 3194 |
| | | mucilaginosa | |
| 171 | 690 | Rhodotorula sp. nearest minuta | UOFS Y-0125 |
| 172 | Jen 31 | Sporidiobolus microsporus | NCYC 3224 |
| 173 | BVO 09 | Sporobolomyces holsaticus | NCYC 3198 |
| 174 | N 15 | Trichosporon beigelii | NCYC 3200 |
| 175 | 232 | Trichosporon cutaneum var. | NCYC 3202 |
| | | cutaneum | |
| 176 | 15 | Trichosporon mucoides | NCYC 3206 |
| 177 | 223 | Trichosporon mucoides | UOFS Y-0116 |
| 178 | 19 | Trichosporon ovoides | NCYC 3207 |
| 179 | 59 | Trichosporon sp. | UOFS Y-0861 |
| 180 | 224 | Trichosporon sp. | UOFS Y-0449 |
| 181 | 225 | Trichosporon sp. | NCYC 3211 |
| 182 | 231 | Trichosporon sp. | NCYC 3210 |

TABLE 11
(samples 183-206). Hydrolysis of trans-1-phenylpropene
oxide by selected yeasts (20% cells, 50 mM epoxide, 3 hours).

| | | | | % |
|---|---|---|---|---|
| | | | | conversion |
| | | | Culture | of 1S,2S- |
| NO | SNO | Yeast Strain | collection no. | epoxide |
| 183 | 15 | Trichosporon mucoides | NCYC 3206 | 90 |
| 184 | 223 | Trichosporon mucoides | UOFS Y-0116 | 84 |
| 185 | 228 | Trichosporon beigelii | UOFS Y-1580 | 65 |
| 186 | 225 | Trichosporon sp. | NCYC 3211 | 50 |
| 187 | 224 | Trichosporon sp. | UOFS Y-0449 | 46 |
| 188 | 232 | Trichosporon cutaneum | NCYC 3202 | 43 |
| | | var. cutaneum | | |
| 189 | 21 | Trichosporon cutaneum | UOFS Y-0063 | 39 |
| | | var. cutaneum | | |
| 200 | 231 | Trichosporon sp. | NCYC 3210 | 37 |
| 201 | 230 | Cryptococcus albidus | NCYC 3156 | 30 |
| 202 | 155 | Trichosporon sp. | NCYC 3212 | 27 |
| 203 | 229 | Trichosporon sp. | UOFS Y-2113 | 25 |
| 204 | 227 | Geotrichum sp. | UOFS Y-0111 | 24 |
| 205 | 50 | Rhodotorula glutinis | NCYC 3186 | 22 |
| 206 | 14 | Trichosporon mucoides | NCYC 3205 | 19 |

Only the (1S,2S)-enantiomer was hydrolysed by these yeasts. No conversion of the (1R,2R) enantiomer was observed. Only yeast strains that converted >20% of the (1S,2S) enantiomer in 3 hours are shown.

TABLE 12
(samples 207-221). Yeast strains exhibiting stereoselectivity for hydrolysis
of indene oxide

| | | | Culture | eeₛ (%) | Conversion |
|---|---|---|---|---|---|
| NO | SNO | Yeats Strain | Collection no | (1R, 2S) | (%) |
| 207 | | Rhodosporidium toruloides | NCYC 3215 | 76.2^{a} | 75.3^{a} |
| 208 | 50 | Rhodotorula glutinis | NCYC 3186 | 61.0^{a} | 70.8^{a} |
| 209 | AB49 | Cryptococcus podzolicus | UOFS Y-1882 | 52.1^{a} | 75.2^{a} |
| 210 | EP230 | Rhodotorula arauntiaca | NCYC 3185 | 36.6^{a} | 80.7^{a} |
| 211 | 681 | Rhodotorula glutinis | UOFS Y-0653 | 30.0% | 94.0^{a} |
| 212 | Car-22 | Rhodotorula glutinis | UOFS Y-2227 | 29% | 63.0^{a} |
| 213 | 25 | Rhodotorula araucariae | NCYC 3183 | 23.2^{a} | 84.3^{a} |
| 214 | POH 20 | Rhodosporidium toruloides | NCYC 3216 | 18.4^{b} | 56.0^{b} |
| 215 | POH-28 | Rhodosporidium toruloides | NCYC 3215 | 17% | 62.0^{b} |
| 216 | Car-38 | Rhodosporidium toruloides | UOFS Y-2228 | 15% | 46.0^{b} |
| 217 | Car-52 | Rhodosporidium toruloides | UOFS Y-2230 | 15% | 48.0^{b} |
| 218 | 185 | Rhodotorula glutinis | UOFS Y-0559 | 8% | 58.0^{a} |
| 219 | Jen 31 | Sporidiobolus salmonicolor | NCYC 3196 | 7.6^{c} | 39.9^{c} |
| 220 | Car 205B | Trichosporon montevideense | NCYC 3225 | 5.8^{a} | 88.9^{a} |
| 221 | 692 | Rhodosporidium paludigenum | NCYC 3179 | 5.4^{b} | 40.1^{c} |

Each culture was grown in YPD liquid media and harvested after 3 days.
^{a}The biotransformations were conducted at 50% wet mass/volume biocatalyst loading in 50 mM Indene oxide, at 25° C. for 1 hour.
^{b}Conducted at 25% wet mass/volume biocatalyst loading at 25° C. for 40 minutes.
^{c}Conducted at 25% wet mass/volume biocatalyst loading at 25° C. for 30 minutes.
All negative controls in absence of biocatalyst showed ‘conversions’ of <5% and ‘ee values’ of <2% initial starting values.

### Example IV

### Production of Recombinant Host Strains Expressing YEIH Sequencing, Cloning and Overexpression of YEIH Coding Sequences in a Production Host

### Microbial Strains, Plasmids and Oligonucleotides

Microbial strains, plasmids and oligonucleotides used in this study are listed in Tables 13, 14 and 15, respectively.

TABLE 13
Microbial trains used in these studies

| | | Source/ |
|---|---|---|
| Strain | Genotype/Description | Reference |
| E. coli XL-10 Gold | Tet^{r} D(mcrA)183 D(mcrCB-hsdSMR-mrr)173 | Stratagene, |
| | endA1 supE44 thi-1 recA1 gyrA96 relA1 lac Hte | USA |
| | [F′ proAB lacl^{q}ZDM15 Tn10 (Tet^{r}) Amy Cam^{r}]. | |
| C. neoformans | #777 | CBS 132 |
| R. mucilaginosa | #23 | UOFS Y-0137 |
| R. araucariae | #25 | NCYC 3183 |
| R. toruloides | #46 | UOFS Y-0471 |
| R. toruloides | #1 | NCYC 3181 |
| R. paludigenum | #692 | NCYC 3179 |
| C. curvatus | # Car 54 | NCYC 3158 |
| Y. lipolytica Po1h | MATA, ura3-302, uxpr2-322, axp1-2) | Madzak et al. |
| | | (2003) |
| YL-23 TsA | Po1h transformed with pYLTsA carrying the | This study |
| | mEH from R. mucilaginosa (UOFS Y-0198). | |
| YL-777 TsA | Po1h transformed with pYLTsA carrying the | This study |
| | mEH from C. neoformans (CBS 132). | |
| YL-25 TsA | Po1h transformed with pYLTsA carrying the | This study |
| | mEH from R. araucariae (NCYC 3183). | |
| YL-46 TsA | Po1h transformed with pYLTsA carrying the | This study |
| | mEH from R. toruloides (UOFS Y-0471 | |
| YL-1 TsA | Po1h transformed with pYLTsA carrying the | This study |
| | mEH from R. toruloides (NCYC 3181) | |
| YL-692 TsA | Po1h transformed with pYLTsA carrying the | This study |
| | mEH from R. paludigenum (NCYC 3179). | |
| YL-Car 54-TsA | Po1h transformed with pYLTsA carrying the | This study |
| | mEH from C. curvatus (NCYC 3158). | |
| YL-23 HmA | Po1h transformed with pYLHmA carrying the | This study |
| | mEH from R. mucilaginosa (UOFS Y-0198). | |
| YL-777 HmA | Po1h transformed with pYLHmA carrying the | This study |
| | mEH from C. neoformans (CBS 132). | |
| YL-25 HmA | Po1h transformed with pYLHmA carrying the | This study |
| | mEH from R. araucariae (NCYC 3183). | |
| YL-46 HmA | Po1h transformed with pYLHmA carrying the | This study |
| | mEH from R. toruloides (UOFS Y-0471 | |
| YL-1 HmA | Po1h transformed with pYLHmA carrying the | This study |
| | mEH from R. toruloides (NCYC 3181) | |
| YL-692 HmA | Po1h transformed with pYLHmA carrying the | This study |
| | mEH from R. paludigenum (NCYC 3179). | |
| YL-Car 54-HmA | Po1h transformed with pYLHmA carrying the | This study |
| | mEH from C. curvatus (NCYC 3158).. | |

TABLE 3
Plasmids used in these studies

| | | Source/ |
|---|---|---|
| Plasmid | Description | Reference |
| pGEM ®-T | General vector containing T overhangs for cloning of | Promega, |
| Easy | adenylated PCR products. | USA |
| pINA1313 | Single copy integrative shuttle vector containing Kan^{R} | Nicaud et al. |
| | and ura3d1 selective markers. Random integration into | (2002) |
| | Po1h genome through the ZETA transposable element. | |
| | The plasmid contains the synthetic promoter, hp4d, and | |
| | the Y. lipolytica LIP2 signal peptide. | |
| pKOV96 = pYLTsA | Single copy Zeta element based integrative shuttle | This study |
| | vector containing Kan^{R} and carrying the non-defective | |
| | ura3d1 selection marker. Similar to pINA1313, with | |
| | hp4d replaced with TEF promoter and Y. lipolytica LIP2 | |
| | signal sequence removed. | |
| pYLHmA = pINA1291 | Multiple copy integrative shuttle vector containing Kan^{R} | Nicaud et al |
| | and ura3d4 selective markers. Random integration into | (2002) |
| | Po1h genome through the ZETA transposable element. | |
| | The plasmid contains the synthetic promoter, hp4d. | |
| pGEM-777 | pGEM ®-T Easy harboring the YEIH ORF from C. neoformans | This study |
| | (CBS 132). | |
| pGEM-23 | pGEM ®-T Easy harboring the mEH ORF from R. mucilaginosa | This study |
| | (UOFS Y-0198). | |
| pGEM-46 | pGEM ®-T Easy harboring the mEH ORF from R. toruloides | This study |
| | (UOFS Y-0471). | |
| pGEM-25 | pGEM ®-T Easy harboring the mEH ORF from R. araucariae | This study |
| | (NCYC 3183). | |
| pGEM-692 | pGEM ®-T Easy harboring the mEH ORF from R. paludigenum | This study |
| | (NCYC 3179). | |
| pGEM-Car | pGEM ®-T Easy harboring the mEH ORF from C. curvatus | This study |
| 54 | (NCYC 3158). | |
| pYL-777- | pYL TsA harboring the EH ORF from C. neoformans | This study |
| TsA | (CBS 132). | |
| pYL-23-TsA | pYL TsA harboring the EH ORF from R. mucilaginosa | This study |
| | (UOFS Y-0198). | |
| pYL-25-TsA | pYL TsA harboring the mEH ORF from R. araucariae | This study |
| | (NCYC 3183). | |
| pYL-46 TsA | pYL TsA harboring the EH ORF from R. toruloides | This study |
| | (UOFS Y-0471). | |
| pYL-1-TsA | pYL TsA harboring the EH ORF from R. toruloides | This study |
| | (NCYC 3181). | |
| pYL-692- | pYL TsA harboring the EH ORF from R. paludigenum | This study |
| TsA | (NCYC 3179). | |
| pYL-Car 54- | pYL TsA harboring the EH ORF from C. curvatus | This study |
| TsA | (NCYC 3158). | |
| pYL-777- | pYLHmA harboring the EH ORF from C. neoformans | This study |
| HmA | (CBS 132). | |
| pYL-23- | pYLHmA harboring the EH ORF from R. mucilaginosa | This study |
| HmA | (UOFS Y-0198). | |
| pYL-25- | pYLHmA harboring the mEH ORF from R. araucariae | This study |
| HmA | (NCYC 3183). | |
| pYL-46 HmA | pYLHmA harboring the EH ORF from R. toruloides | This study |
| | (UOFS Y-0471). | |
| pYL-1-HmA | pYLHmA harboring the EH ORF from R. toruloides | This study |
| | (NCYC 3181). | |
| pYL-692- | pYLHmA harboring the EH ORF from R. paludigenum | This study |
| HmA | (NCYC 3179). | |
| pYL-Car 54- | pYLHmA harboring the EH ORF from C. curvatus | This study |
| HmA | (NCYC 3158). | |

TABLE 4

Oligonucleotide primers used in these studies

| | | Restric- |
|---|---|---|
| | | tion |
| | | sites |
| | Sequence in 5′ to 3′ | Intro- |
| Primer Name | orientation | duced |
| C. | TGG ATC CAT GTC GTA TTC AGA | BamHI |
| neoformans- | CCT TCC CC | |
| 1F | (SEQ ID NO: 15) | |
| C. | TGC TAG CTC AGT AAT TAC CTT | NheI |
| neoformans- | TGT ACT TCT CCC AC | |
| 1R | (SEQ ID NO: 16) | |
| R. | AGA TCT ATG CCC GCC CGC TCG | BglII |
| mucilaginosa- | CTC | |
| 1F | (SEQ ID NO: 17) | |
| R. | TCC TAG GCT ACG ATT TTT GCT | AvrII |
| mucilaginosa- | CCT GAG AGA GAG | |
| 1R | (SEQ ID NO: 18) | |
| R. | GTGGATCCATGGCGACACACA | BamHI |
| toruloides- | (SEQ ID NO: 19) | |
| 1F | | |
| R. | GACCTAGGCTACTTCTCCCACA | AvrII/ |
| toruloides- | (SEQ ID NO: 20) | BlnI |
| 1R | | |
| R. | GATTAATGATCAATGAGCGAGCA | BclI |
| araucariae- | (SEQ ID NO: 21) | |
| 1F | | |
| R. | GACCTAGGTCACGACGACAG | BlnI |
| araucariae- | (SEQ ID NO: 22) | |
| 1R | | |
| R. | GTGGATCCATGGCTGCCCA | BamHI |
| paludigenum- | (SEQ ID NO: 23) | |
| 1F | | |
| R. | GAGCTAGCTCAGGCCTGG | NheI |
| paludigenum- | (SEQ ID NO: 24) | |
| 1R | | |
| Car 54.-1F | GTGGATCCATGGCGACACACA | BamHI |
| | (SEQ ID NO: 25) | |
| Car 54-1R | GACCTAGGCTACTTCTCCCACA | AvrII |
| | (SEQ ID NO: 26) | |
| Integration- | CCTAGGGTGTCTGTGGTATCTAAGC | Integra- |
| 1F | (SEQ ID NO: 27) | tion |
| | | screening |
| | | for Po1h |
| Integration- | CCGTCTCCGGGAGCTGC | Integra- |
| 1R | (SEQ ID NO: 28) | tion |
| | | screening |
| | | for Po1h |
| pINA-1 | CATACAACCACACACATCCA | Integra- |
| | (SEQ ID NO: 29) | tion |
| | | screening |
| | | for Po1h |
| pINA-2 | TAAATAGCTTAGATACCACAG | Integra- |
| | (SEQ ID NO: 30) | tion |
| | | screening |
| | | for Po1h |

### Recombinant DNA Techniques

### General Genetic Techniques

Standard genetic techniques were used according to Sambrook and Russell (2001). Restriction and modifying enzymes were obtained from Fermentas Life Sciences (Burlington, Ontario, Canada). pGEM®-T Easy (Promega, Madison, Wis., U.S.A.) and pcrSMART™ (Lucigen, Middleton, Wis., U.S.A.) vectors were used for cloning of adenylated and non-adenylated PCR products, respectively. PCR screening for correct integration was performed using Taq DNA polymerase (New England Biolabs, Ipswich, Mass., U.S.A.). PCR and gel band purification was performed using the GFX™ PCR DNA and Gel Band Purification Kit (Amersham Biosciences, Piscataway, N.J., U.S.A.).

Nucleic acid concentrations were determined using a Eppendorf BioPhotometer (Eppendorf, AG, Hamburg, Germany).

Polymerase chain reactions (PCRs) were carried out, unless otherwise stated, using Expand High Fidelity (EHF) PCR system (Roche Molecular Biochemicals, Mannheim, Germany) according to the manufacturer's recommendations. The reaction mixture contained 5 μl of the 10×EHF buffer containing 15 mM MgCl₂, 300 nM upstream and downstream primers, 200 μM dNTPs, 0.5 μg template DNA, 2.6 units of the EHF enzyme mix, filled to a final volume of 50 μl using sterile redistilled water.

Thermal cycling was performed using an Eppendorf Mastercycler Personal (Eppendorf AG) with the following cycling program, unless otherwise stated: initial denaturation of 5 min at 94° C., 40 cycles of denaturation (94° C. for 15 seq), annealing (primer dependent for 30 see), elongation (72° C. for 90 seq with extended elongation time of 5 seq per cycle starting from cycle 10). Final elongation of 10 min at 72° C. was performed to complete elongation of the amplified product.

All PCR and DNA products were electrophoresed and assessed on a 1% (w/v) agarose gel containing 2.5 mg/μl ethiduim bromide. The agarose gels were prepared and electrophoresed in TAE buffer [0.1 M Tris, 0.05 M EDTA (pH 8.0) and 0.1 mM glacial acetic acid] at 5.6 V/cm for 45 min. DNA was visualized under a high radiation UV source, while DNA to be isolated from agarose gels for further studies was visualized using a low radiation UV source prior to isolation.

Transformation of modified plasmid vectors were performed as follows: 80 μl of competent Top 10® E. coli cells (Inoue et al., 1990) were transformed with the ligation mixture containing the coding sequence of interest ligated into a suitable amplification vector. The transformation was performed as described by Sambrook et al. (1989) and the cells were plated onto LB plates supplemented with ampicillin (60 mg/I), IPTG [isopropylthio-β-galactoside (10 mg/I)] and X-gal [5-bromo-4-chloro-3-indoly-β-D-galactoside (40 mg/I)]. Plates were incubated at 37° C. for 16 hours. Positive transformants were selected and inoculated into 5 ml LB-media supplemented with ampicillin (10 μl/ml). Transformants were allowed to grow for 16 hours while shaking at 37° C.

DNA mini-preparations were performed using the lysis by boiling method (Sambrook et al., 1989). Screening for the correct recombinant plasmids was performed using restriction analysis and/or sequence analysis. DNA from positive clones were purified using the GFX™ PCR DNA and gel band purification kit (Amersham) and eluted into 50 μl elution buffer (10 mM Tris-HCl, pH 8.5).

The purified plasmids were used in sequencing reactions to determine the nucleotide composition of the various coding sequences. The plasmids were sequenced using the ABI Prism® Big Dye™ Terminator Cycle Sequencing Ready Reaction Kit v. 3.0 or 3.1 (Applied Biosystems, Foster City, Calif., USA) according to the manufacturer's instructions. Approximately 30-50% of each sequencing reaction was loaded onto a 4% acrylamide gel, separated at 1.6 kV and data collected on an ABI Prism 377 DNA Sequencer (Perkin Elmer, Wellesly, Mass., USA). The data was analyzed using Sequencing Analysis v. 3.3 (Perkin Elmer). Sequences were assembled using AutoAssembler v. 3.3 (Perkin Elmer). Reverse-complementation and alignments were done using DNAssist v. 2.0 (Perkin Elmer).

### Media and Growth Conditions

Luria-Bertani (LB) broth (Sambrook et al., 1989) was used for Escherichia coli cultivation. The cells were cultivated at 37° C. with aeration under selective pressure with 60 μg/ml ampicillin or 50 μg/ml kanamycin in liquid and solid media when required.

Y. lipolytica, R. toruloides, R. paludigenum, R. araucariae and R. glutinis cultures were maintained on solid YPD medium or grown in YPD broth cultures containing yeast extract (10 g/l), peptone (10 g/l) and glucose (20 g/l) or supplemented with agar (1.5 g/l) for solid media. For induction experiments cells were grown in GPP medium (50 mM phosphate buffer (pH 7.5), glycerol (1%, v/v), proteose peptone (0.34%), adenine (50 mg/I) and yeast nitrogen base (0.34%, without amino acids and ammonium sulfate).

For the transformants carrying the hp4d^{p} promoter, cells were cultivated in YPD medium in shake flasks at 28° C.

Selection of transformants using URA3 as auxotrophic marker was performed using YNB casamino acids medium [YNB without amino acids, ammonium sulphate (1.7 g/l), NH₄Cl (4 g/l), glucose (20 g/l), casamino acids (2 g/l), and agar (15 g/l)] supplemented with 300 mg/l leucine).

Construction of pYLHmA, a Multi-Copy Integrative Vector without a Secretion Signal (HmA Transformants)

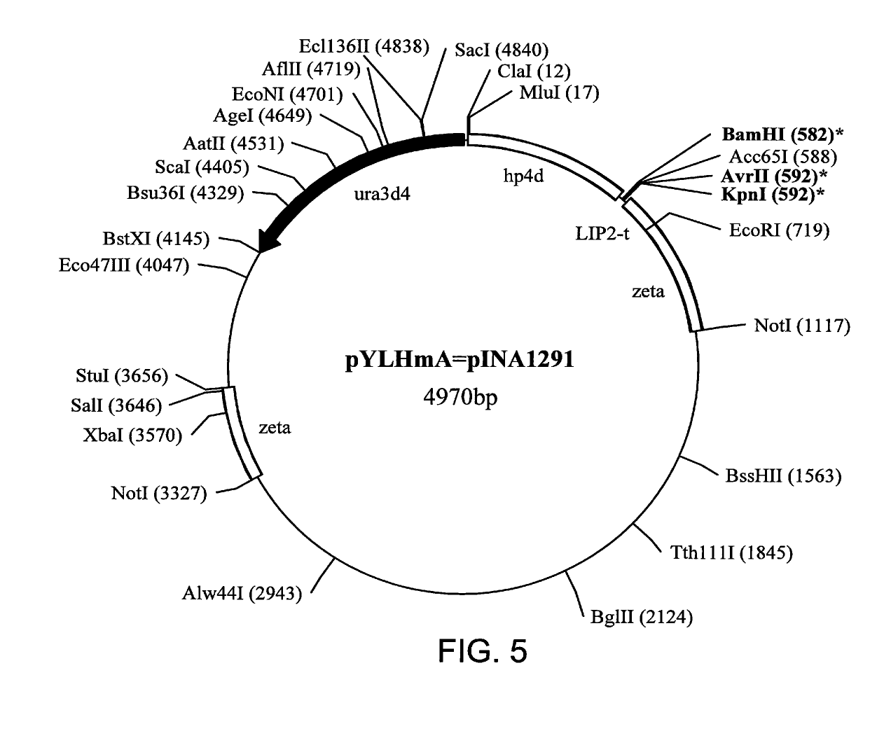

pINA1291 (FIG. 5) was obtained from Dr. Catherine Madzak of labo de Génétique, INRA, CNRS, France. This plasmid was renamed pYLHmA (Yarrowia Lipolytica expression vector, with Hp4d promoter, Multi-copy integration selection, A=no secretion signal)

Construction of a Single-Copy Plasmid (pYL-TsA) Containing the Constitutive TEF^{p} and No Signal Peptide

The quasi-constitutive hp4d promoter (Madzak et al., 2000) was replaced with the constitutive TEF promoter (Möller et al., 1998) in the mono-integrative plasmid pINA1313 (Nicaud et al., 2002). The use of the TEF promoter aided in the activity screening experiments, since the hp4d promoter is growth phase dependent (only active from early stationary phase), whereas the TEF promoter drives constitutive expression to limit induction differences between yeasts grown during activity screening and on flask scale.

The hp4d promoter in pINA1313 was replaced with the TEF promoter using ClaI and HindIII restriction sites, followed by the PCR removal of the LIP2 signal peptide using primers-sigP-1F and -sigP-1R. The purified PCR mixture was treated with BamHI and HindIII (where HindIII digested the template DNA but not the PCR product) to prevent recircularization of the template DNA, thereby preventing concomitant template contamination of transformation mix upon ligation. The PCR product was allowed to circularize using T4 DNA ligase to join the compatible BamHI ends resulting in plasmid pKOV96=pYLTsA (FIG. 6).

The YEIH coding sequences of #1, #23, #25, #46, #692 & #777 and Candida albicans (Ca) were amplified using the primers in Table 3.

The amplified YEIH coding sequences and the pKOV96=pYLTsA plasmid were digested with the appropriate restriction enzymes to create compatible cohesive ends suitable for ligation of the YEIH coding sequences into the BamHI-AvrII cloning sites of the plasmid, resulting in plasmids pYL1-TsA, pYL-23TsA, pYL-25TsA, pYL-46TsA pYL-692TsA, pYL777-TsA and pYL-Ca-TsA.

### Nucleic Acid Isolation, Amplification, Cloning and Sequencing of Epoxide Hydrolase Encoding Coding Sequences

Yeast strains were obtained from the UOFS yeast culture collection and were cultivated in 50 ml YPD media (20 g/l peptone; 20 g/l glucose; 10 g/l yeast extract) at 30° C. for 48 hours while shaking. Cells were harvested by centrifugation and the subsequent pellet was either frozen at −70° C. for RNA isolation or suspended to a final concentration of 20% (w/v) in 50 mM phosphate buffer (pH 7.5) containing 20% (v/v) glycerol and frozen at −70° C. for DNA isolation.

DNA isolation entailed addition of 500 μl lysis solution (100 mM Tris-HCl, pH 8.0; 50 mM EDTA, pH 8.0; 1% SDS) and 200 μl glass beads (425-600 μm diameter) to 0.4 g wet cells, followed by vortexing for 4 min, cooling on ice and addition of 275 μl ammonium acetate (7 M, pH 7.0). After incubation at 65° C. for 5 min followed by 5 min on ice, 500 μl chloroform was added, vortexed and centrifuged (20 000×g, 2 min, 4° C.). The supernatant was transferred and the DNA precipitated for 5 min at room temperature using 1 volume iso-propanol and centrifuged (20 000×g, 5 min, 4° C.). The pellet was washed with 70% (v/v) ethanol, dried and re-dissolved in 100 μl TE (10 mM Tris-HCl; 1 mM EDTA, pH 8.0).

Total RNA isolation entailed grinding 10 g wet cells under liquid nitrogen to a fine powder, 0.5 ml of the powder was added to a pre-cooled 1.5 ml polypropylene tube and thawed by the addition of TRIzol® solution (Invitrogen). The isolation of total RNA using TRIzol® was performed according to the manufacturer's instructions. The total RNA isolated was suspended in 50 μl formamide and frozen at −70° C. for further use.

Reverse transcription of total RNA into cDNA was performed as follows. To isolate the cDNA sequence, primers were designed according to the sequence data available and used in a two step RT-PCR reaction as follows. First strand cDNA synthesis was performed on total RNA using Expand Reverse Transcriptase (Roche Applied Science) in combination with primer Rm cDNA-2R at 42° C. for 1 hour followed by heat inactivation for 2 minutes at 95° C. The newly synthesized cDNA was amplified using primers Rm cDNA-2F and Rm cDNA-1R (initial denaturation for 2 minutes at 94° C.; followed by 30 cycles of 94° C. for 30 sec; 67° C. for 30 sec; 72° C. for 2 min and a final elongation of 72° C. for 7 min).

Forward and reverse primers (Table 15) were designed to introduce the required restriction sites during PCR to allow for subcloning of the EH encoding coding sequences into the multi-coy vector pYL-HmA. All YEIH encoding coding sequences were PCR amplified using Expand High Fidelity Plus PCR System (Roche Applied Sciences). Thermal cycling entailed initial denaturation of 2 min at 94° C. followed by 30 cycles of 94° C. for 30 sec, Tₘ−5° C. for 30 sec (Tₘ was calculated using the modified nearest neighbor calculation obtained from Integrated DNA Technologies, Coralville, Iowa, U.S.A.; www.idtdna.com) and 72° C. for 2 min. A 10 min at 72° C. final elongation step was included to allow complete synthesis of amplified DNA. PCR products were gel purified and cloned into pGEM®-T Easy.

Vectors containing the YEIH encoding coding sequences of interest were transformed into XL-10 Gold® E. coli for plasmid amplification and sequencing. The YEIH encoding coding sequences were subjected to restriction and sequence analysis (sequencing performed by Inqaba Biotechnical Industries) before transfer of the coding sequences from the cloning vectors to the expression vectors.

The cloning vectors containing the YEIH encoding coding sequences were treated with the restriction enzyme pairs indicated in Table 15, to liberate the YEIH encoding coding sequences. The liberated fragments were ligated into BamHI and AvrII linearized pYLHmA expression vectors.

### Transformation and Selection of Multiple Copy Transformants (YL-HmA Transformants)

Y. lipolytica Po1 h cells were transformed with NotI linearized pYL-HmA vector containing the YEIH encoding coding sequences (according to the method described by Xuan et al., 1988) and plated onto YNB_{casa} plates [YNB without amino acids and ammonium sulfate (1.7 g/l), ammonium chloride (4 g/l), glucose (20 g/l), casamino acids (2 g/l) and agar (15 g/l)].

Transformants were subjected to genomic DNA isolation, followed by PCR screening to confirm presence of the integrated NotI-expression cassette. This entailed amplification of a ˜1.6 kb fragment using primers pINA-1 and pINA-2 in a standard PCR (annealing at 50° C.).

Po1 h transformants that tested positive for activity were inoculated into 200 ml YPD and incubated while shaking at 28° C. for 48 hours (stationary phase). Cells were harvested by centrifugation (6 000×g for 5 min), washed with and resuspended in 50 mM phosphate buffer (pH 7.5) containing 20% glycerol (v/v) to a final concentration of 50% (w/v) and stored at −20° C. for future experiments.

### Example III

### Production of Optically Active trans-2,3-disubstituted Epoxides and Vicinal Diols

This example illustrates the use of a wild type yeast strain selected from Trichosporon genus and recombinant host strains transformed with YEIH coding sequences to produce optically active epoxides and vicinal diols from trans-1-phenylpropene oxide.

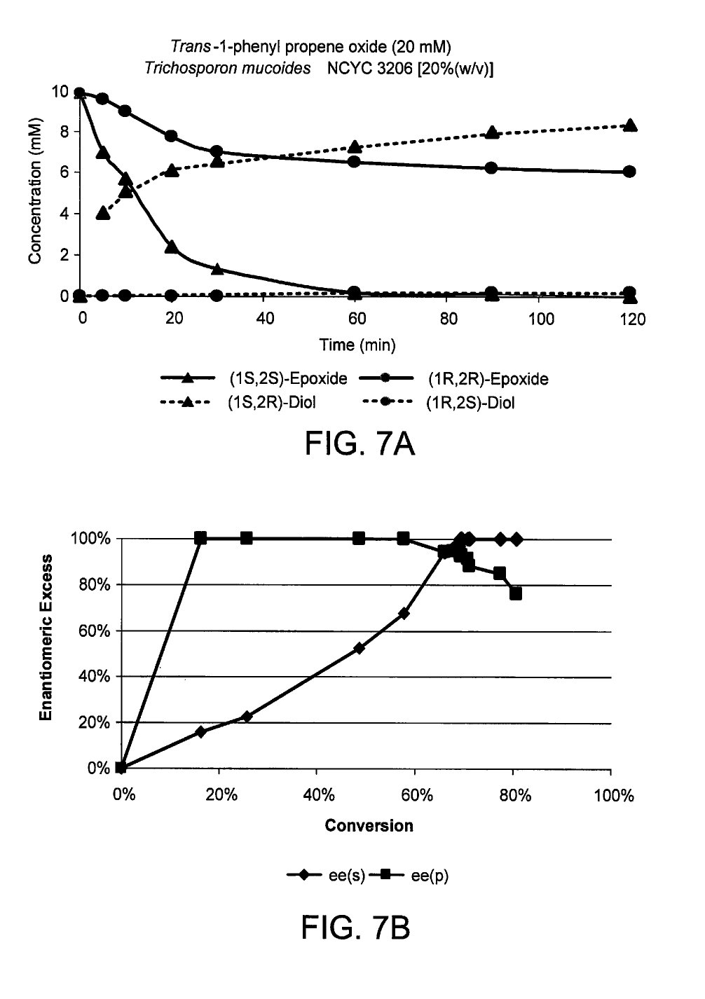

FIG. 7A shows the change in concentrations of the epoxide and diol enantiomers with time during the hydrolysis of trans-1-phenylpropene oxide by Trichosporon mucoides NCYC 3206. FIG. 7B shows the enantiomeric excess of the epoxide and diol at different conversions. The yield of the optically active epoxide or diol that can be obtained at the required enantiomeric purity can be seen from these graphs.

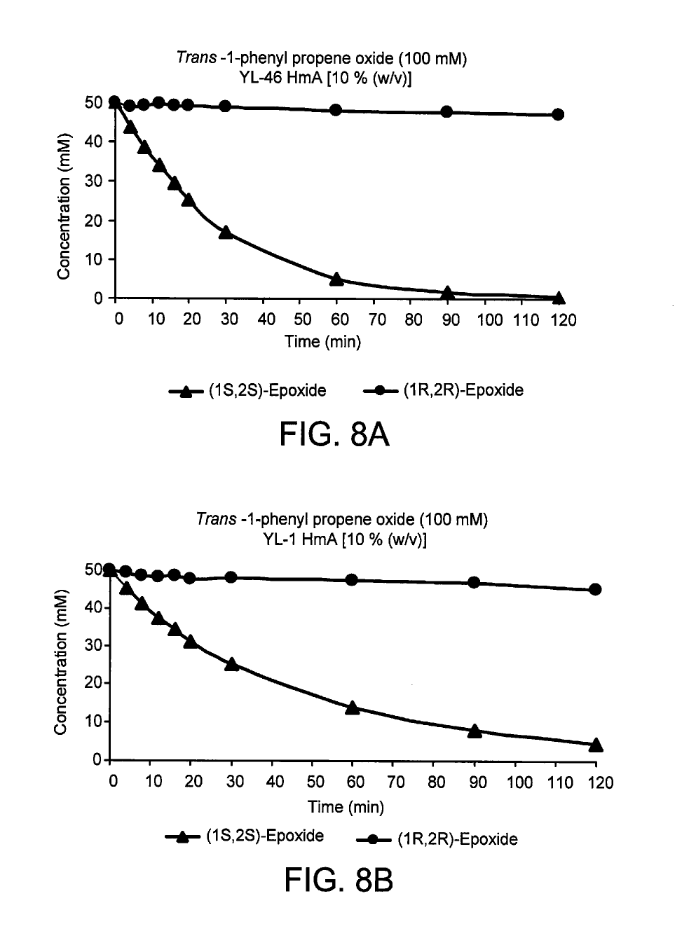

FIG. 8 shows two samples of the hydrolysis of trans-1-phenyl propene oxide by recombinant host strains YL-46 HmA and YL-1 HmA respectively, expressing the YEIH from Rhodosporidium toruloides strains (#46=UOFS Y-0471, FIG. 8A) and #1=NCYC 3181, FIG. 8B).

During the screening of different wild type strains for the hydrolysis of trans-1-phenyl propene oxide enantiomers, Rhodosporidium toruloides strains displayed such low activity compared to Trichosporon mucoides (Table 11) that they were not selected as examples for Table 11. However, when these YEIH were expressed in the host, the hydrolysis of racemic trans-1-phenyl propene oxide proceeded with excellent activity and selectivity. Expression of the YEIH in the host strain thus caused a marked improvement in the hydrolysis rate, while the excellent enantioselectivity displayed by the wild type strains was maintained.

### Example IV

### Production of optically active (R,R)-2,3-buanediol from cis-2,3-epoxybutane

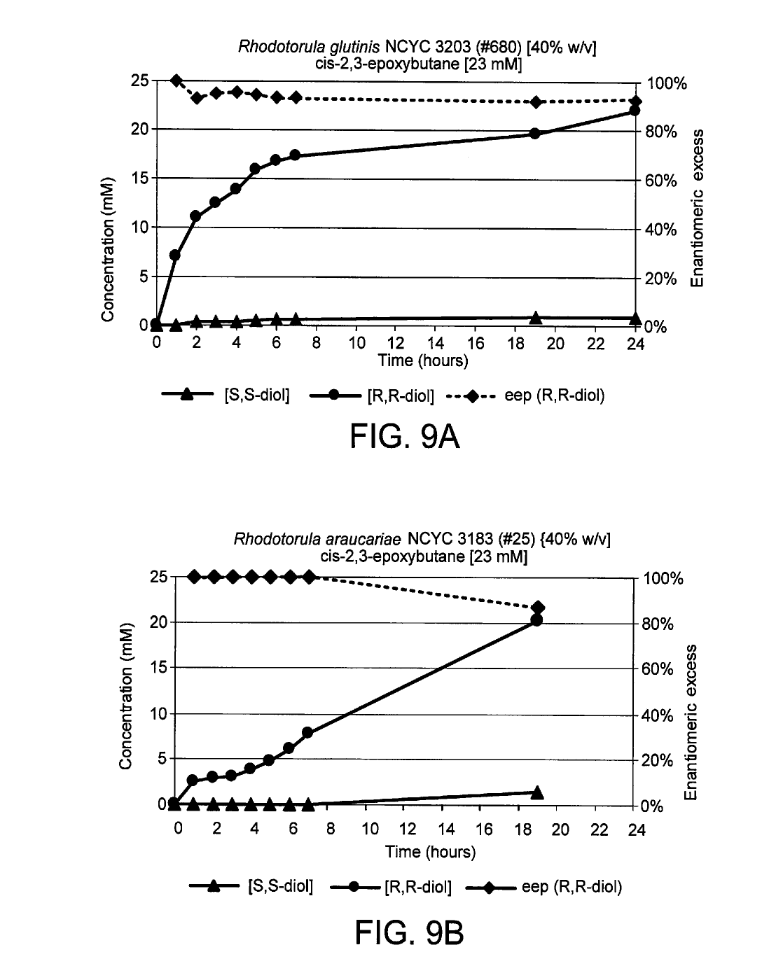

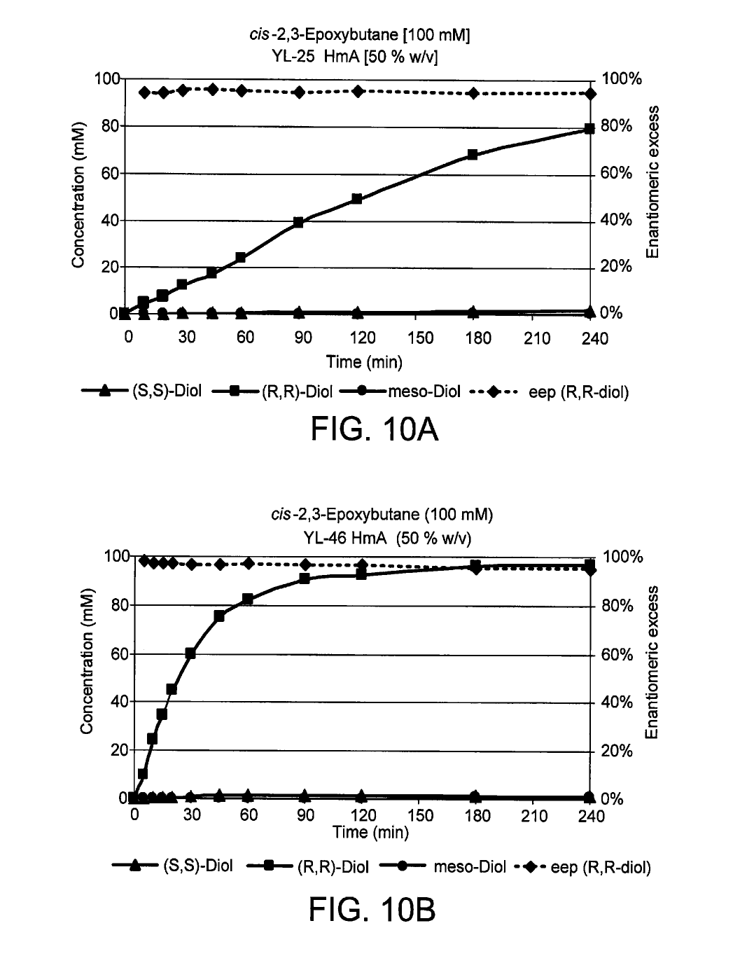

Strains were selected from Table 7 based on the ee's observed during screening and used for biotransformation reactions that were monitored over time. Selected reactions of the hydrolysis of cis-2,3-epoxybutane by wild type yeast strains are shown in FIG. 9A and FIG. 9B. The ee of the formed diols were high (>80% in most cases). Significant enhancement of reaction rates and higher enantiomeric excesses (>95%) were achieved using recombinant host strains overexpressing the YEIH, examples of which are shown in FIGS. 10A and 10B.

### Example V

### Production of Optically Active (1R,2S)-Indene Oxide and (1R,2R) Indanediol from Racemic Indene Oxide by Wild Type Yeast Strains

Over 300 wild type yeasts were grown up and screened as described in Example 1. The yeasts were isolated from the UOFS culture collection (University of Free State, Bloemfontein, Republic of South Africa) and were selected based on the ability to metabolise terpene type substrates. Approximately 95% of the yeasts screened showed hydrolytic activity on racemic indene oxide Budged by diol formation on TLC). Approximately 20% of the active yeast strains screened showed enantioselective hydrolysis activity and all the activities observed resulted in accumulation of the (1R,2S) epoxide enantiomer, opposite to the selectivity observed by Chartraih (U.S. Pat. No. 5,849,568). Table 12 illustrates the yeasts exhibiting highest enantiomeric selectivity on 50 mM indene oxide.

The wild type yeasts were grown in shake flask culture and harvested as described above. The protocol followed for the screen was as follows:
To 500 ul of wild type cell suspension [cell pellet from flask culture suspended to 50% m/v (wet mass) in 50 mM phosphate buffer, pH 7.5, 20% w/v glycerol] in 1.5 ml Eppendorf tubes, 5 ul from a 5.5 M substrate stock solution (in EtOH) was added to give a final indene oxide concentration of 50 mM.
The tubes were incubated at 25° C. for 1 hour on an Eppendorf shaker.
The reactions were stopped by the addition of 300 ul of hexane, vortexed for 1 minute, centrifuged at 13 000 rpm f or 5 min.
The hexane layer was transferred into HPLC vials for chiral analysis.

Some reactions were repeated over longer or shorter times and with more dilute cell suspensions (10% w/v) in order to analyse the reactions at suitable conversions. Activity for indene oxide was present in most of the 260 yeast strains tested. Selectivity of the wild type strains was only moderate under the reaction conditions used. Of the yeast genera screened, Rhodosporidium, Rhodotorula, Cryptococcus, Sporidiobolus and Trichosporon showed the greatest potential as catalysts for the resolution of racemic indene oxide although several other yeast genera such as Bretanomyces and Dekkera also showed some enantioselectivity.

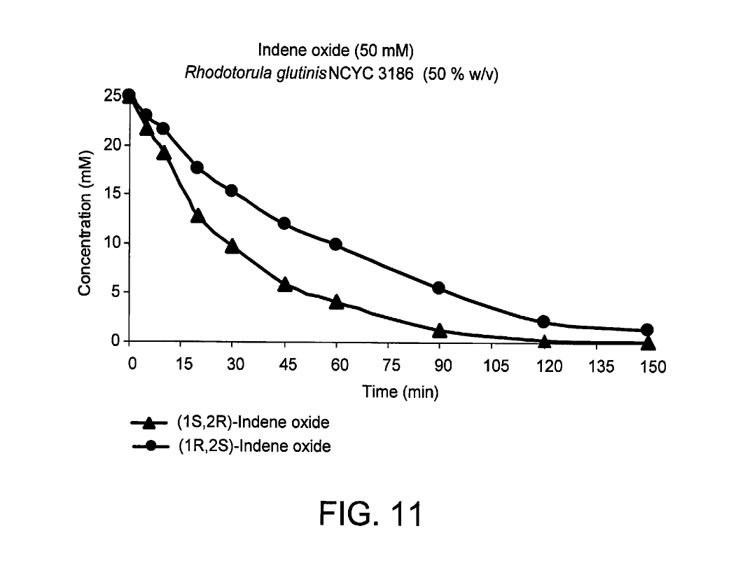

A representative example of the hydrolysis of indene oxide by Rhodotorula glutinis and Rhodoporidium toruloides strains is given in FIG. 11 that shows the hydrolysis of indene oxide by Rhodotorula glutinis NCYC 3186.

Rhodotorula and Rhodosporidium species were chosen as examples for further evaluation in recombinant overexpression hosts. Rhodosporidium paludigenum NCYC 3179 was specifically included as it displayed the worst performance of the yeasts selected (Table 12).

### Example VI

### Small Scale Biotransformations at 100 mM (12 g/l) Racemic Indene Oxide Substrate Loading Using Whole Cell Biocatalyst Comprising Recombinant Yarrowia lipolytica Strains Expressing the YEIH Coding Sequences Isolated from Rhodosporidium and Rhodotorula Strains under Different Promoters

For the purposes of the studies described herein, useful polypeptides exhibiting epoxide hydrolase activity on indene oxide have been over-expressed in Yarrowia lipolytica yeast host and examples of biocatalyst production and biotransformation resolutions of indene oxide using recombinant Yarrowia lipolytica are provided.

Recombinant YL-HmA and YL-TsA strains were produced by integrative insertion of a polypeptide exhibiting epoxide hydrolase activity isolated from wild type strains and cloned into Yarrowia lipolytica under two different promoter systems (TEF and hp4d promoters) as described in Example 3.

YL-HmA and YL-TsA transformants were cultivated at 25° C. in 250 ml shake flasks containing 50 ml sterilised liquid rich media, the latter comprising yeast extract (5 g·L⁻¹), malt extract (20 g·L⁻¹), peptone (10 g·L⁻¹) and glucose (15 g·L⁻¹). After 48 hours, biomass was harvested by centrifugation (3000 rpm, 15 min, 5° C.) and resuspended to 10% (w/v) in phosphate buffer [50 mM, pH 7.5, containing 20% (v/v) glycerol] and frozen until required.

Of each YL-TsA transformant, 5 ml of a 10% (w/v) whole cell suspension and of each YL-HmA transformant, 5 ml of a 5% (W/v) whole cell suspension (prepared as described above) was dispensed into a glass reaction vial fitted with a screw cap and a rubber septum, and stabilised at 20° C. for 30 min. A 2 M stock solution of indene oxide (synthesised in-house Example 1) in absolute ethanol was prepared. Reactions were started by addition of 250 μl of the substrate stock, yielding a final concentration of 12 g/l. The reactions were incubated at 20° C. on a magnetic stirrer for 60 min, stirring at 500 rpm. Samples (300 μl) were taken regularly, extracted with 500 μl hexane, vortexed for 30 sec and centrifuged at 13000 rpm for 3 min, after which the organic layers were removed, dried over anhydrous MgSO₄ and analysed by chiral high performance liquid chromatography (HPLC). Determination of IE concentration(s) and enantiomeric excess was achieved using a Chiralcel OB-H column, using hexane:ethanol (90:10) mobile phase, at constant flow rate of 1 ml·min⁻¹. The maximum yield achievable is 50% based on a racemate starting substrate.

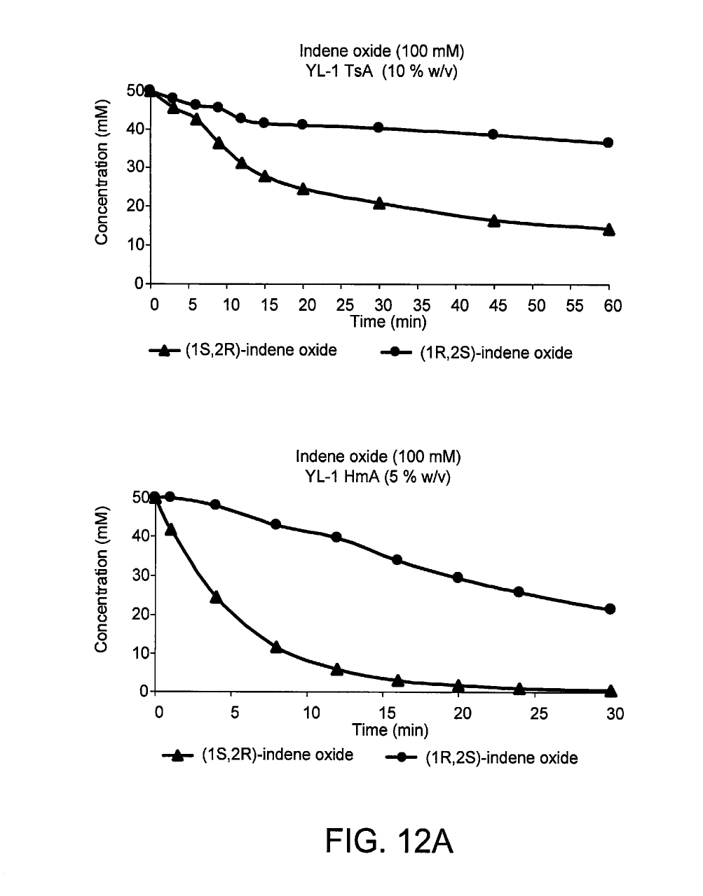

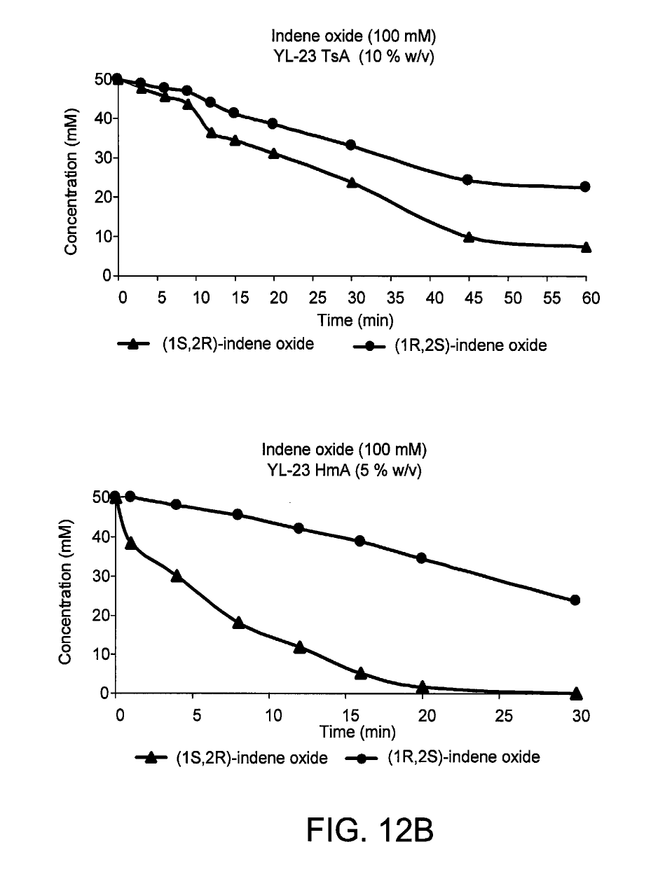

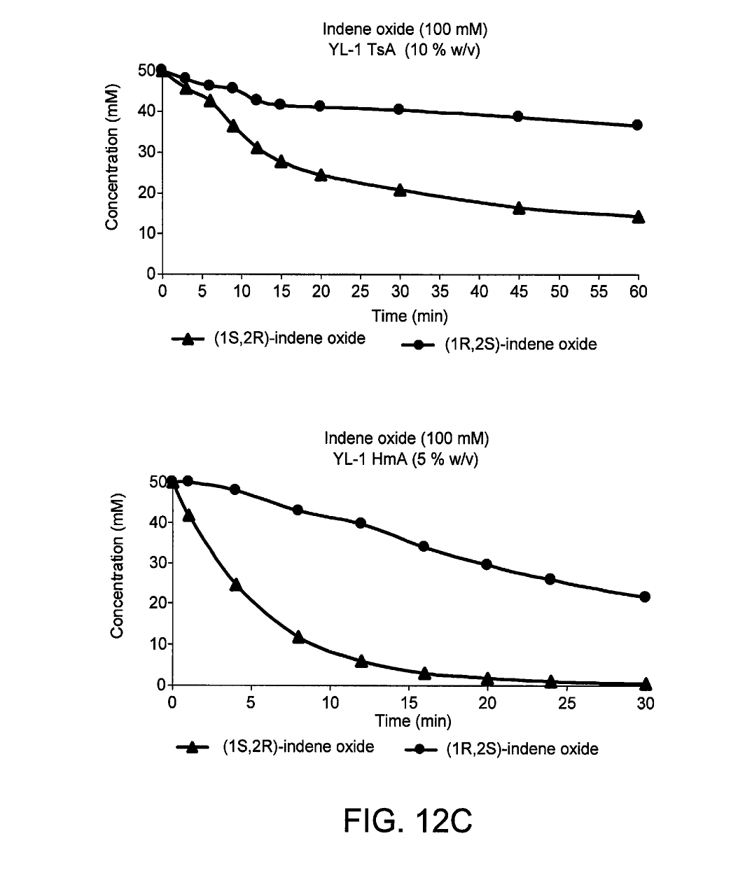

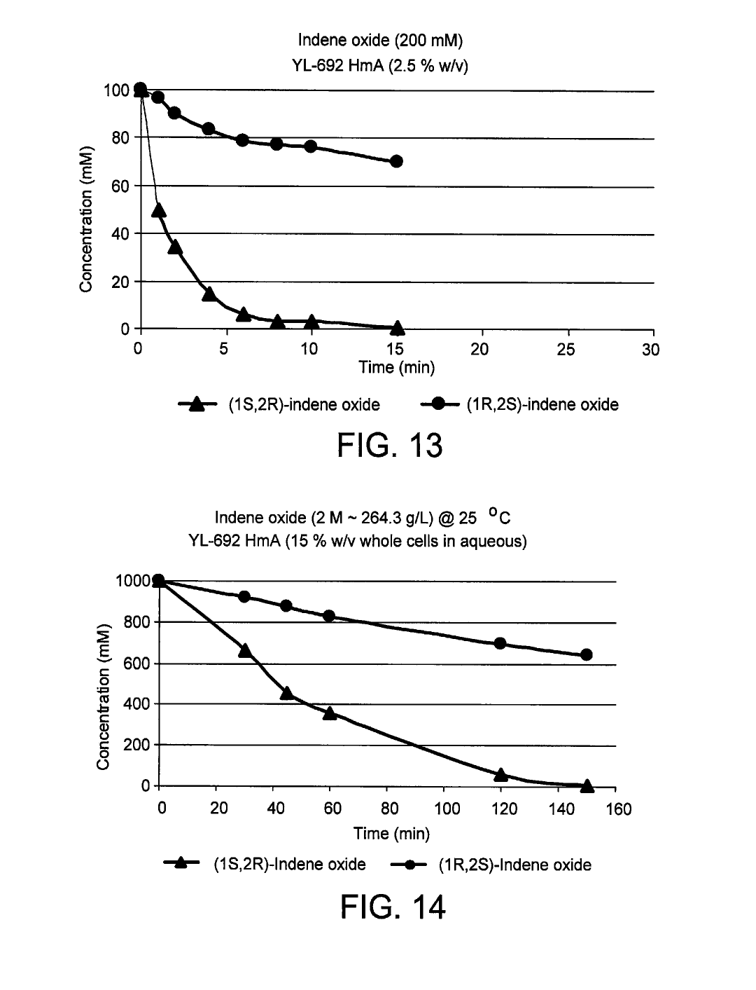

The hydrolysis profiles of indene oxide by the different YL-TsA and YL-HmA transformants that displayed the best activity and selectivity are given in FIG. 12 A (YL-1 TsA & YL-1 HmA), 12B (YL-23 TsA and YL-23 HmA) and 12C(YL-692 TsA and YL-692 HmA). In all cases, the YL-HmA transformants displayed higher activities than the YL-TsA transformants (Table 13) and complete resolution of racemic indene oxide under the reaction conditions used to produce (1R,2S)-indene oxide enantiomerically pure (ee>98%) was only achieved by the multi-copy YL-HmA transformants, except for Rhodopordium paludigenum NCYC 3179 where resolution was also achieved with the YL-TsA transformant. Surprisingly, YL-transformants expressing the YEIH coding sequence from Rhodopordium paludigenum NCYC 3179 displayed the highest activity and selectivity, in contrast to the wild type screening results where this strain displayed the least interesting activity and selectivity of the strains tested. For YL-692 HmA, the hydrolysis reaction conducted at 100 mM indene oxide and 5% cell loading resulted in a hydrolysis rate that was too fast to follow the time-course reaction reliably (data not shown). The reaction was thus repeated at double the substrate concentration (200 mM), and was still completed in 5 minutes. The reaction was thus repeated at 2.5% mass/volume cell loading (equivalent to <0.5% dry weight catalyst) (FIG. 13) in order to obtain an accurate initial rate of hydrolysis (Table 13).

TABLE 13
Biotransformation of 12 g/l indene oxide by Yarrowia lipolytica
transformants expressing YEIH coding sequences of
Rhodosporidium sp. and Rhodotorula sp.

| | | | Yield of (1R,2S)- |
|---|---|---|---|
| | Activity | | indene oxide |
| YL | (nmol · min⁻¹ · | Final ee | @ >98% ee |
| Transformant | mg wet wt⁻¹)^{a} | (%)^{b} | (%) |
| YL-1 TsA | 20 | 44.1% | n.d. |
| YL-1 HmA | 100 | 98.0% | 25 |
| YL-23 TsA | 11 | 50.5% | n.d. |
| YL-23 HmA | 83 | 98.0% | 30 |
| YL-25 TsA | 10 | 55.7% | n.d. |
| YL-25 HmA | 112 | 98.0% | 18 |
| YL-46 TsA | 15 | 57.9% | n.d. |
| YL-46 HmA | 66 | 64.4% | n.d. |
| YL-692 TsA | 25 | 98.0% | 38 |
| YL-692 HmA^{c} | 1470 | 98.0% | 35 |

^{a}Initial rate of indene oxide hydrolysis
^{b}Percentage after 60 minutes reaction duration with 10% wet mass/volume cell suspension for YL-TsA transformants and 5% wet mass/volume cell suspension for YL-HmA transformants at pH 7.5, 20° C. at 12 g/l indene oxide initial substrate loading.
^{c}2.5% wet mass/volume cell suspension and 24 g/l substrate loading.
n.d., not determined

As evident from Table 13 and FIG. 12, both YL-TsA and YL-HmA transformants expressing the YEIH from Rhodoporidium paludigenum (#692) resulted residual (1R,2S) indene oxide at >98% ee with yields >35% out of a possible maximum theoretical yield of 50% at the point of ee>98%.

The YL-692 HmA transformant was thus used for preparative scale biotransformations at 2 M indene oxide (264 g/L) in the subsequent example.

### Example VII

### Preparative Scale Biotransformations at 264 g/l (2 M) Substrate Loading Using Whole Cell Biocatalyst Comprising Recombinant Yarrowia lipolytica Strain YL-692HmA Expressing the Epoxide Hydrolase Coding Sequence Isolated from Rhodosporidium paludigenum NCYC 3179

YL-692 HmA transformant was cultivated at 25° C. in 250 ml shake flasks containing 50 ml sterilised liquid rich media, the latter comprising yeast extract (5 g·L⁻¹), malt extract (20 g·L⁻¹), peptone (10 g. L¹) and glucose (15 g. L¹). After 48 hours, biomass was harvested by centrifugation (3000 rpm, 15 min, 5° C.) and resuspended to 10% (w/v) in phosphate buffer [50 mM, pH 7.5, containing 20% (v/v) glycerol] and frozen until required.

13.5 gram wet weight whole cell suspension of YL-692HmA, (prepared as described above) was dispensed into 80 ml buffer in a glass reaction vial fitted with a screw cap and a rubber septum, and stabilised at 25° C. for 30 min. Crystalline indene oxide powder (synthesised in-house as described in Example 1) was added to a final concentration of 2 M (264 gram epoxide per litre) by addition of 26.4 g substrate to the 80 ml cell suspension and adjustment of the final reaction volume to 100 ml. The reaction was incubated on a temperature controlled magnetic stirrer at 25° C. for 300 minutes, stirring at 500 rpm. Samples (500 ul) were taken at regular intervals, extracted with 9 ml hexane, vortexed for 30 sec and centrifuged at 13000 rpm for 3 min, after which the organic layers were removed, dried over anhydrous MgSO₄ and analysed by chiral high performance liquid chromatography (HPLC).

Determination of IE concentration(s) and enantiomeric excess was achieved using a Chiralcel OB-H column, using hexane:ethanol (90:10) as the mobile phase, at a constant flow rate of 1 ml·min⁻¹. The maximum yield achievable is 50% based on a racemate starting substrate.

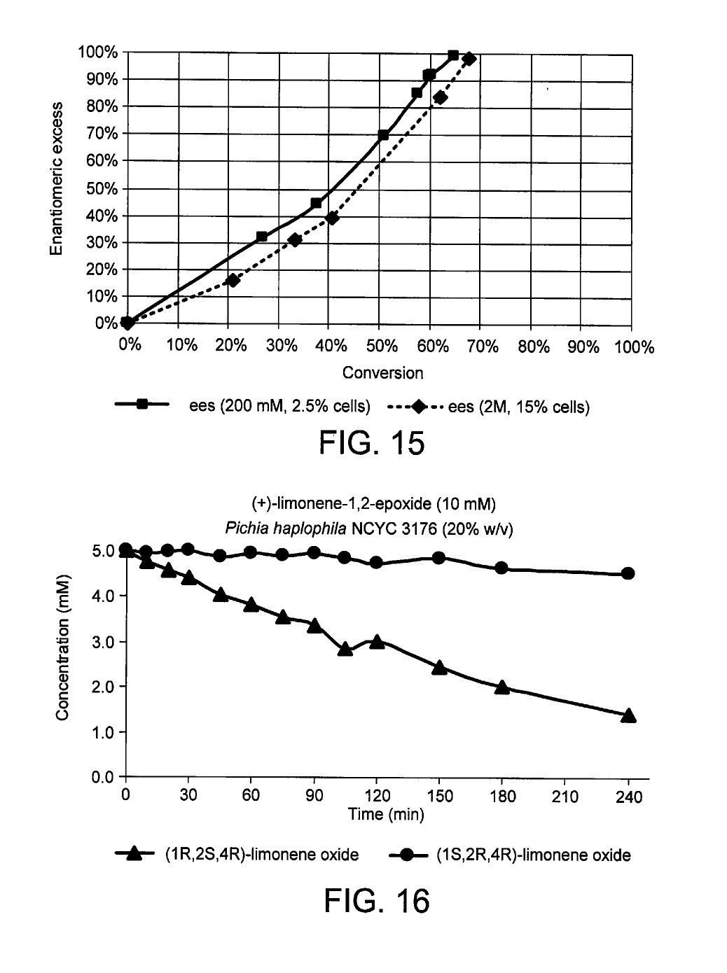

FIG. 14 shows that the reaction was completed in 150 minutes with 32% yield of 1R,2S indene oxide at >98% ee on a substrate loading of 2 mol/litre, significantly higher than all previous reports. Good correlation was achieved between the enantiomeric excesses at different conversions between the small scale (24 g/l) and larger scale (264 g/L) reactions (FIG. 15) showing that the reaction can be scaled up without loss of selectivity. While the yield of homochiral epoxide was only moderate in this example (32%) compared to a theoretical maximum of 50%, those skilled in the art know of well-established methods for increasing the enantioselectivity of an enzyme. The homochiral indene oxide can be recovered by standard downstream methods and the stereochemistry of the isolated epoxide inverted by standard chemical methods in order to form (1S,2R) indene oxide for use as an API in synthesis of Indinavir. Given that the choice of the YEIH coding sequence from wild type isolate NCYC 3179 is not the optimum based on the wild type screen results described in Table 12, it is expected that more suitable YEIH can be cloned from other yeast strains such as Rhodotorula glutinis NCYC 3215.

### Example VIII

### Production of Optically Active Tri-Substituted Epoxides and ID

### (a) (+)-limonene-1,2-epoxide

Pichia haplophila NCYC 3176 (20% w/v) was incubated with 10 mM (+)-limonene-1,2-epoxide (4R, mixture of cis and trans) and the progress of the reaction was monitored over time as described in Example 1 (FIG. 16). The hydrolysis proceeded with excellent selectivity and activity. Epoxide hydrolases from Pichia haplophila strains are thus very useful catalysts for the resolution of tri-substituted epoxides as exemplified by (+)-limonene-1,2-epoxide.

### (b) 6,7-epoxygeranyl-1-ol

YL-HmA transformants (10% w/v) were incubated for 1 hour with the free alcohol of 6,7-epoxygeranyl-1-ol (100 mM) as well as with the corresponding esters containing various protective groups of the alcohol, such as acetate and N-phenyl carbamate. The IE was hydrolysed to the corresponding ID with excellent selectivity by Yarrowia lipolytica transformants expressing epoxide hydrolases from Rhodosporidium toruloides strains #1 (NCYC 3181) and #46 (UOFS Y-0471), as well as from Rhodotorula strains #25 and #692 (R. araucariae NCYC 3183 and R. paludigenum NCYC 3179). All reactions were stopped at 50% conversion, and yielded an enantiomerically pure 6,7-epoxygeranyl-1-ol (or acetate). The enantio-identifications of the residual IE and the ID product were not elucidated.

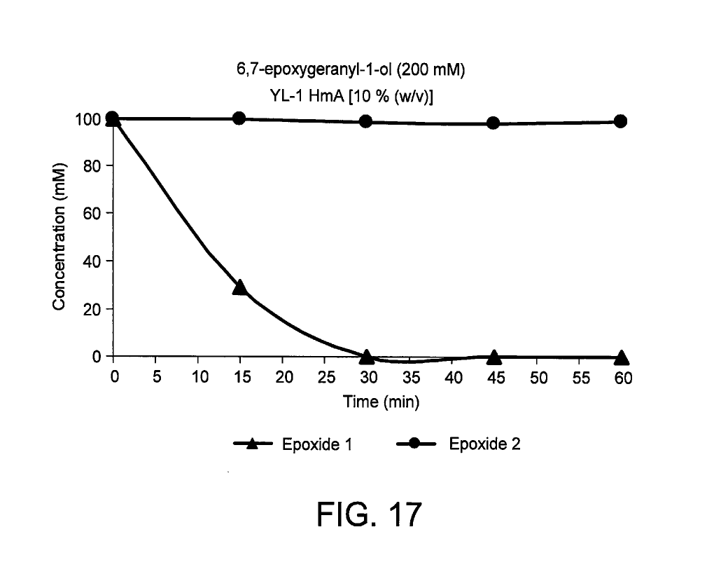

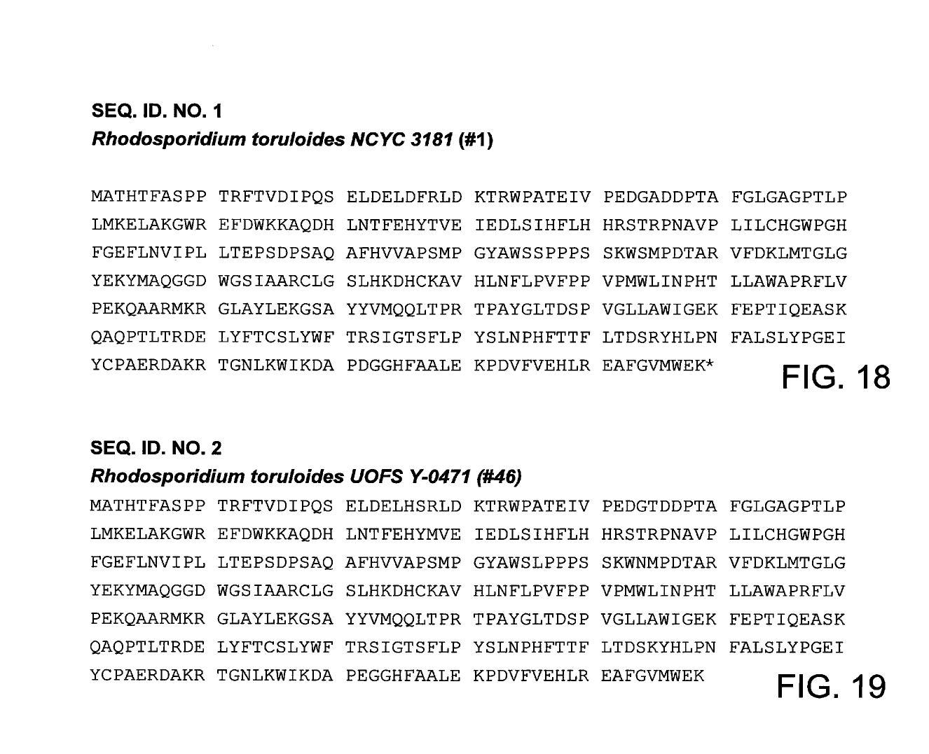

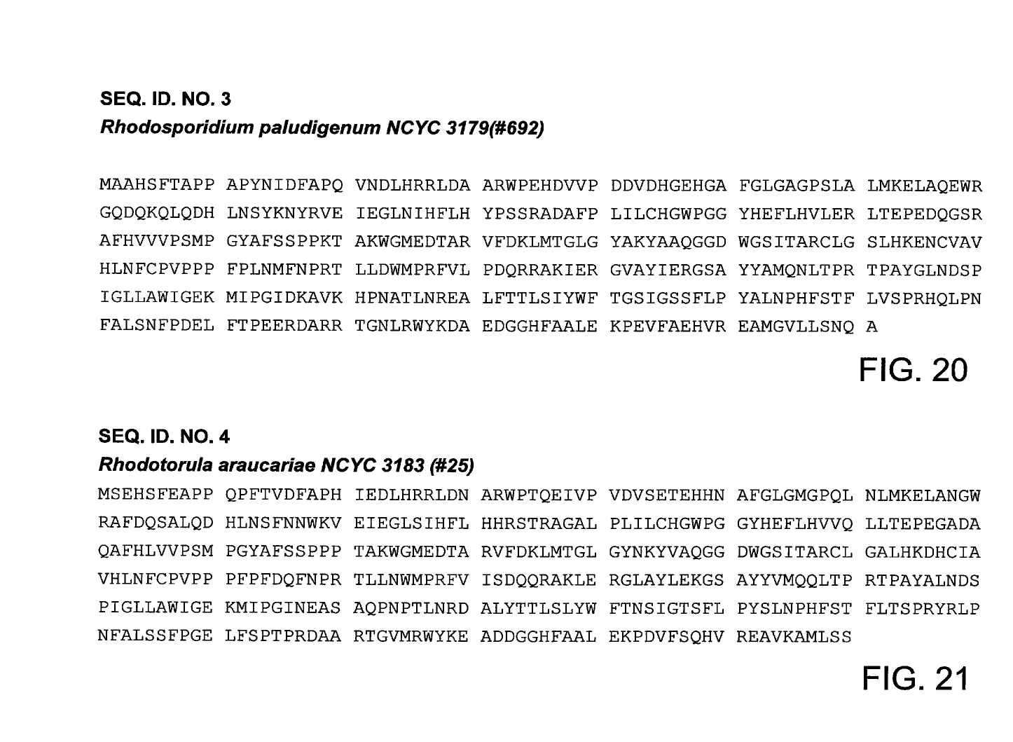

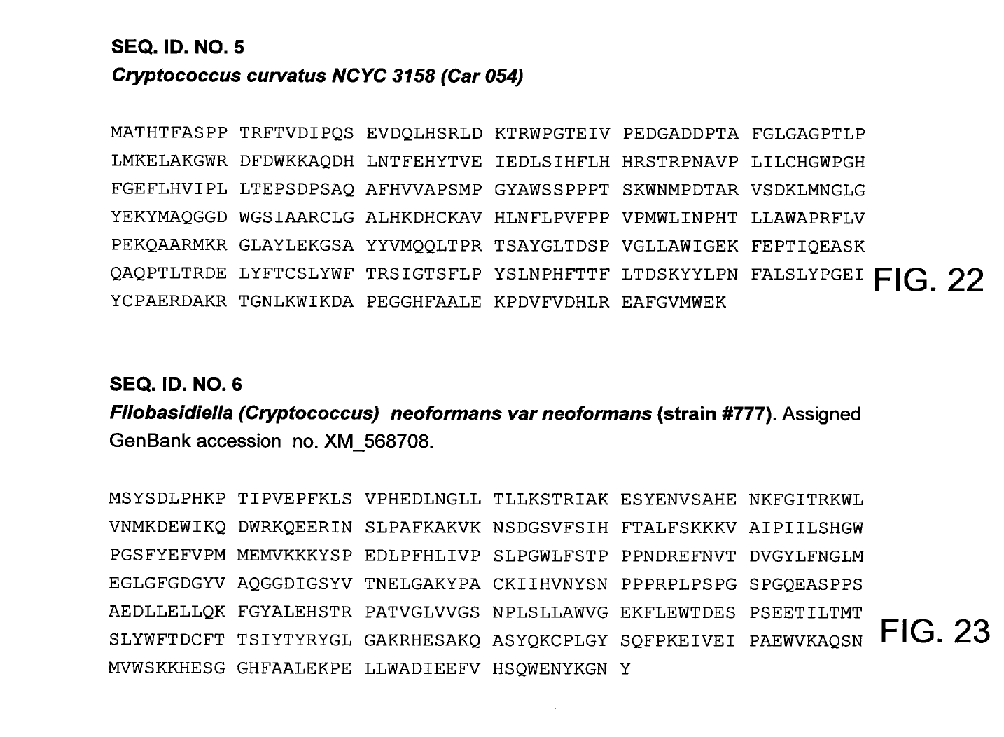

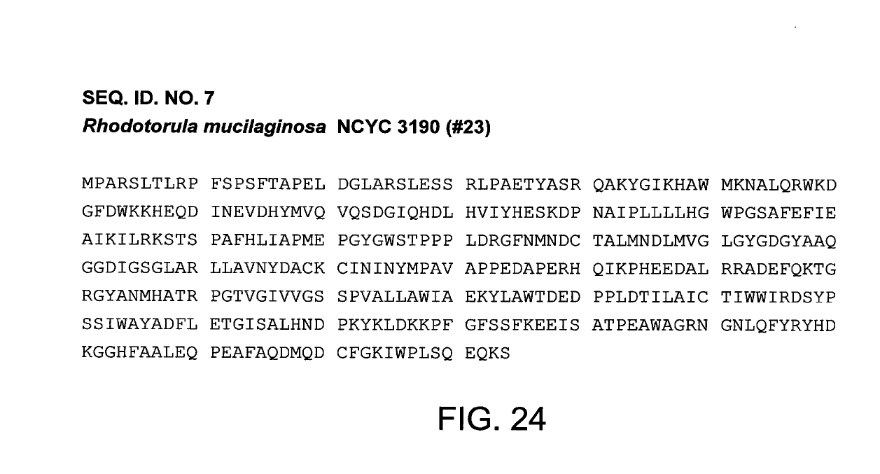

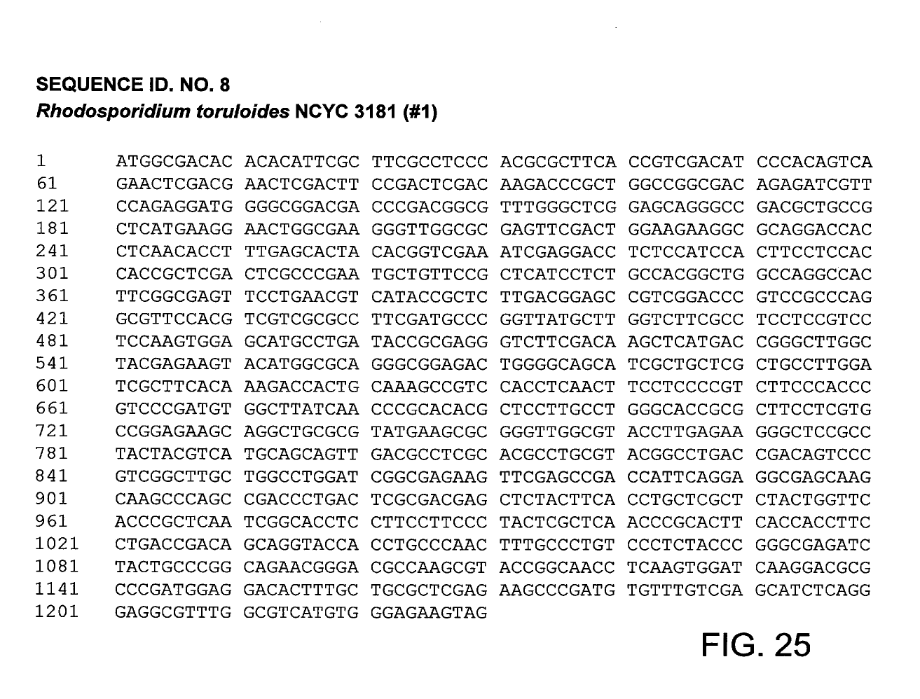

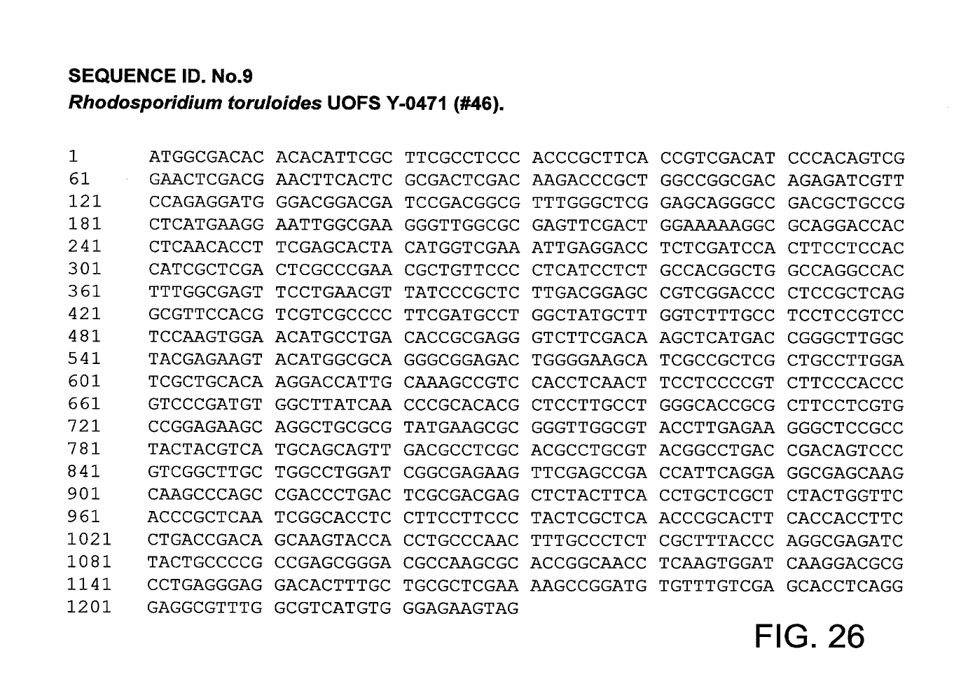

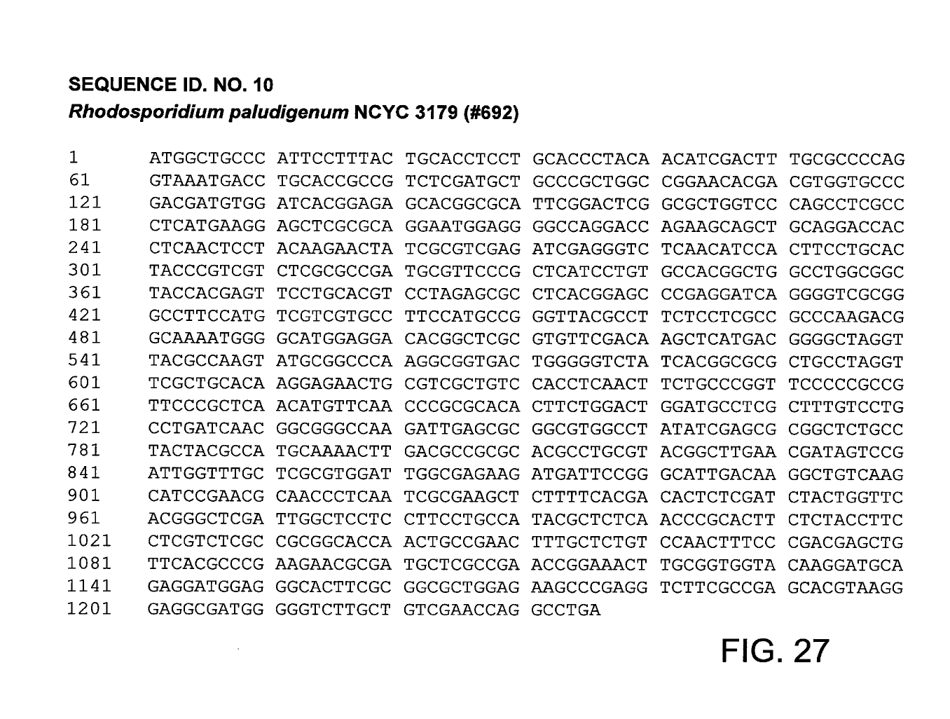

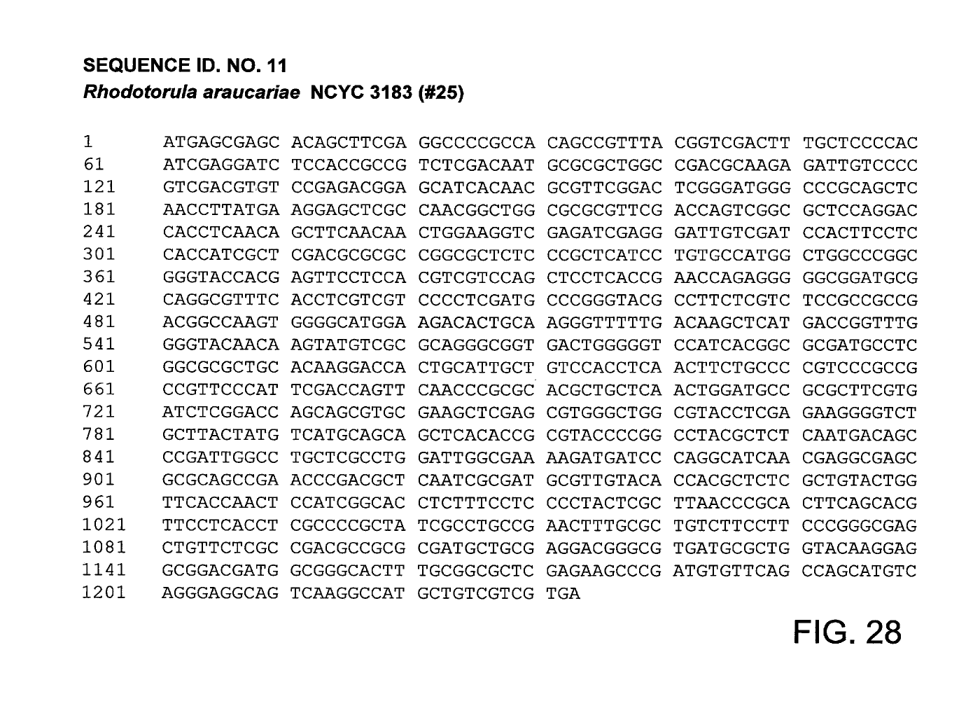

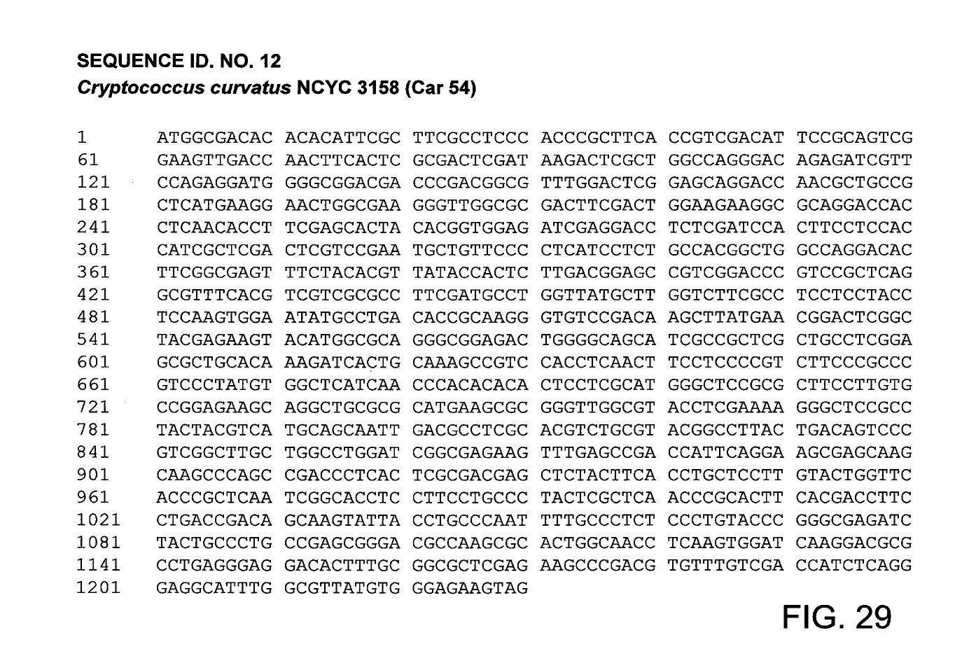

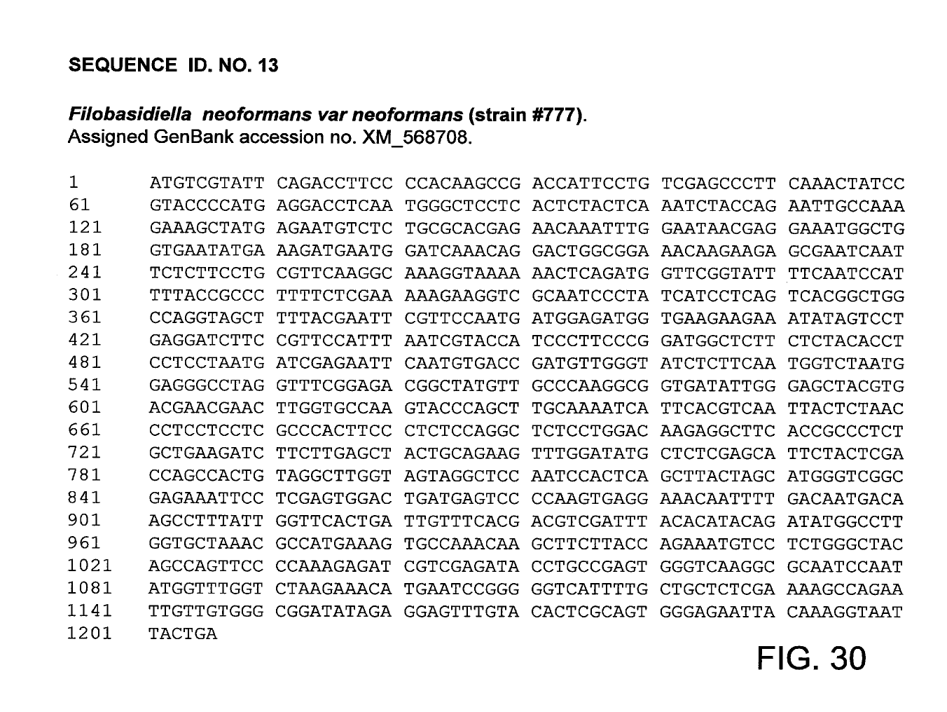

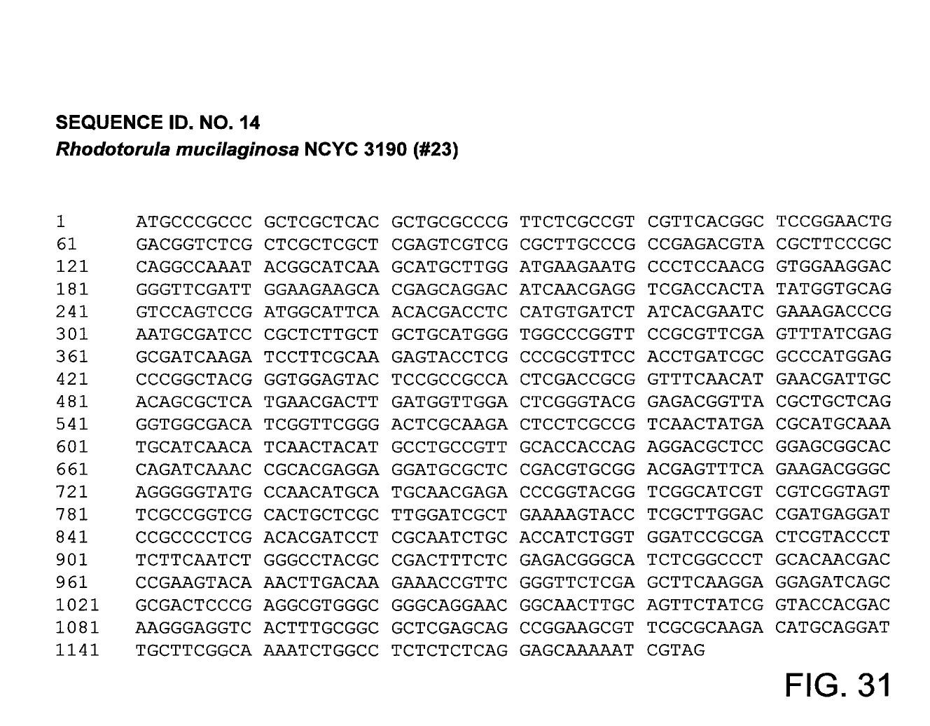

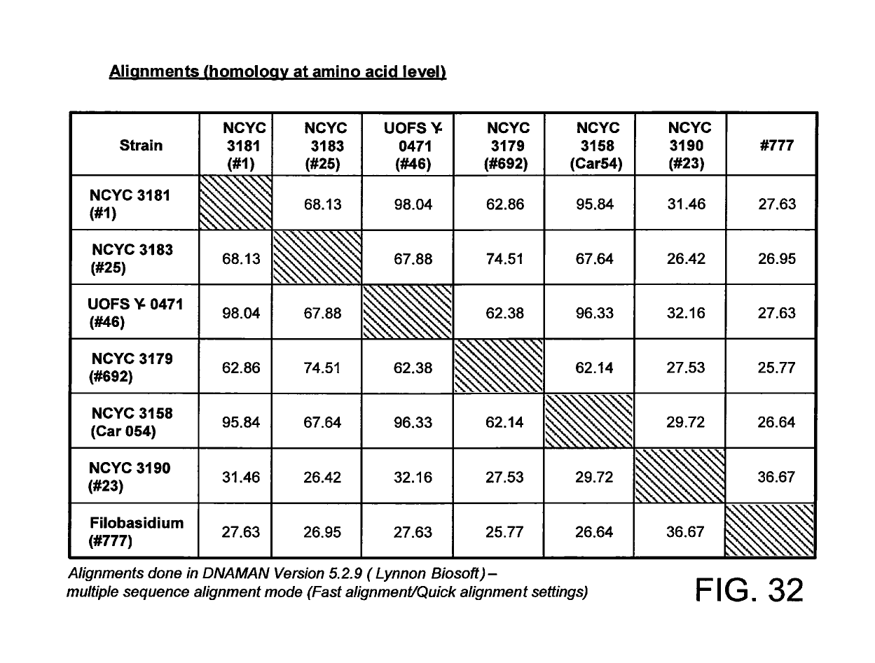

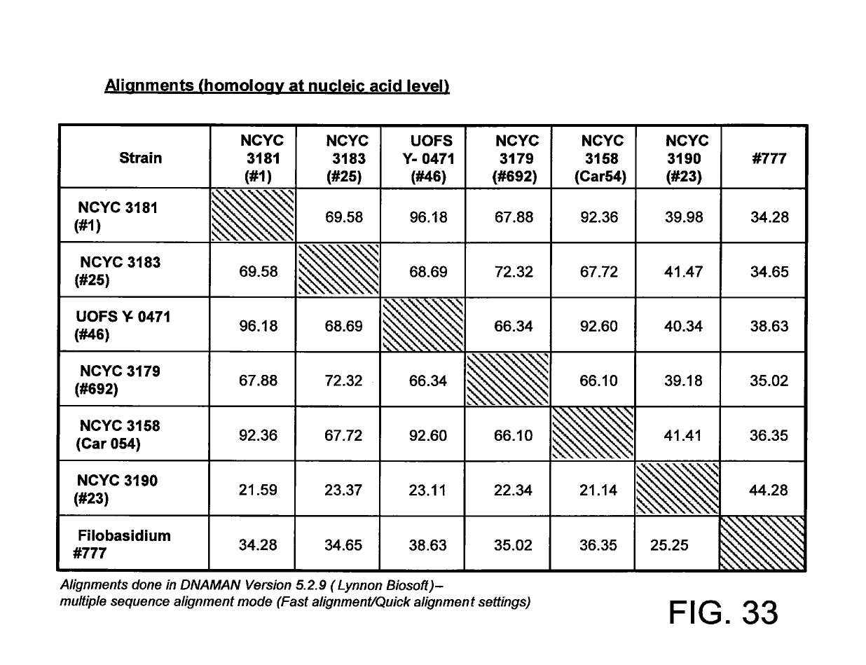

FIG. 17 shows the typical hydrolysis profile obtained for the hydrolysis of 6,7-epoxygeranyl-1-ol by these strains. Under the reaction conditions used (200 mM epoxide substrate, 10% cells), the resolution is typically completed after 15-30 minutes for all strains tested.

### Example IX

### Production of Optically Active Methylene Interrupted Bis-Epoxides and Diols

Yeast cells (20% w/v) were incubated with linoleic acid bis-epoxide (50 mM) for 1 hour and the reactions analysed by tlc for diol formation. Table 10 lists the yeast strains that are able to hydrolyse linoleic acid bisepoxide with the formation of the THF diol and tetraol (FIG. 3). Reaction mixtures of those strains that displayed activity, were derivatised (methylated with diazomethane followed by silylation) and subjected to non-chiral GC/MS analysis to identify and quantify the products. Enantiomer analysis was not done on the THF diols, but the diastereoisomers and positional isomers were identified as shown in FIGS. 2, 3 and 4 for Rhodotorula glutinis UOFS Y-0123. Similar profiles were obtained for the other yeasts listed in Table 10.

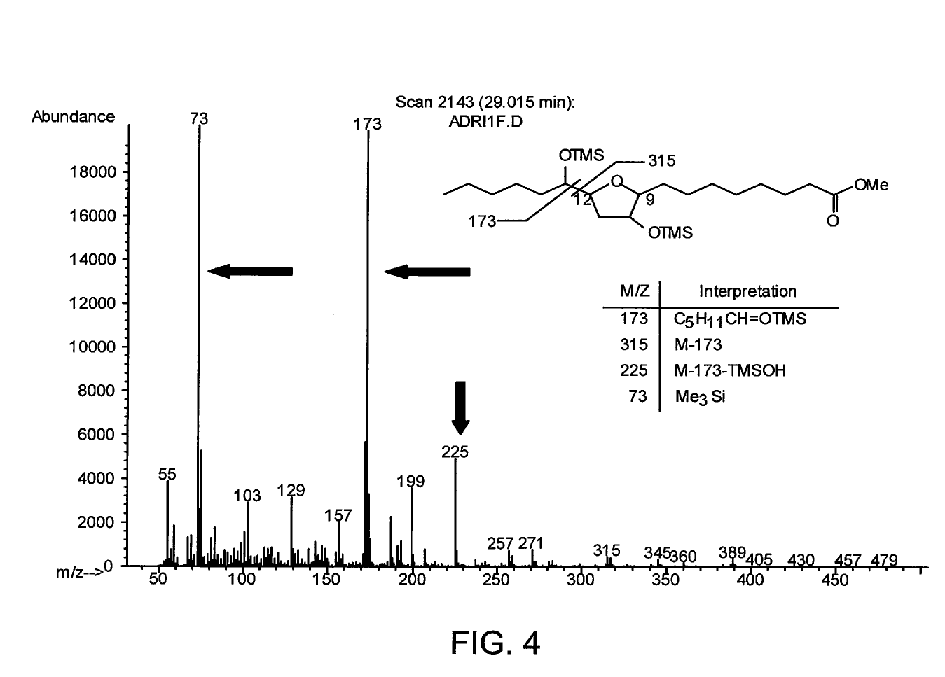

FIG. 2 shows typical GC/MS profiles of the reaction mixture of the hydrolysis of linoleic acid bisepoxide by yeasts listed in Table 10 after methylation and silylation (Example: Rhodotorula glutinis UOFS Y-0123). The numbers of the peaks correspond to the products assigned in FIG. 3.
FIG. 3 shows typical products formed during the hydrolysis of linoleic acid bisepeoxide by the yeasts listed in Table 10. (Example: Rhodotorula glutinis UOFS Y-0123);
FIG. 4 shows MS data of major peak formed during hydrolysis of linoleic acid bisepeoxide: 10,13-dihydroxy-9 (12)-oxyoctadecanoate.

### REFERENCES

Orru, R. V. A., Archelas, A., Furstoss, R. and Faber, K. (1999). Epoxide hydrolases and their synthetic applications. Advances in Biochemical Engineering/Biotechnology, 63, 145-167.
Madzak, C., Tréton, B. and Blanchin-Roland, S. (2000). Strong hybrid promoters and integrative expression/secretion vectors for quasi-constitutive expression of heterologous proteins in the yeast Yarrowia lipolytica. Journal of Molecular Microbiology and Biotechnology 2, 207-216.
Müller, S., Sandal, T., Kamp-Hansen, P., and Dalbøge, H. (1998). Comparison of expression systems in the yeasts Saccharomyces cerevisiae, Hansenula polymorpha, Klyveromyces lactis, Schizosaccharomyces pombe and Yarrowia lipolytica. Cloning of two novel promoters from Yarrowia lipolytica. Yeast 14, 1267-1283.
Nicaud J.-M., Madzak C., Van den Broek P., Gysler C., Duboc P., Niederberger P., Gaillardin C. (2002). Protein expression and secretion in the yeast Yarrowia lipolytica. FEMS Yeast Research 2, 371-279
Watson, J. D., Hopkins, N. H., Roberts, J. W., Steitz, J. A. and Weiner, A. M. (1987). Molecular Biology of the Gene, 4^{th} ed. Benjamin Cummings, Menlo Park, Calif.
Sambrook, J. and Russel. (2001). Molecular cloning. A laboratory manual vol. 1 (3^{rd} ed.). Cold Spring Harbor Laboratory Press. Cold Spring Harbor, N.Y.
Le Dall, M-T., Nicaud, J-M. and Gaillardin, C. (1994). Multi-copy integration in the yeasts Yarrowia lipolytica. Current Genetics 26, 38-44.
Capon, R. J. and Barrow, R. A. (1998). Acid mediated conversion of methylene-interrupted bisepoxides to tetrahydrofurans: A biomimetic transformation. J. Org. Chem., 6 3: 75-83.

A number of embodiments of the invention have been described. Nevertheless, it will be understood that various modifications can be made without departing from the spirit and scope of the invention. Accordingly, other embodiments are within the scope of the following claims.

1. A process for obtaining an optically active internal epoxide (IE) or an optically active internal diol (ID), which process includes the steps of:
providing an enantiomeric mixture of an IE;
creating a reaction mixture by adding to the enantiomeric mixture a polypeptide, or a functional fragment thereof, having enantioselective IE hydrolase activity, the polypeptide being a polypeptide encoded by a gene of a yeast cell;
incubating the reaction mixture; and
recovering from the reaction mixture: (a) an enantiopure, or a substantially enantiopure, ID; (b) an enantiopure, or a substantially enantiopure, IE; or (c) an enantiopure, or a substantially enantiopure, ID and an enantiopure, or a substantially enantiopure, IE.

2. A process for obtaining an optically active internal epoxide (IE) or an optically active internal diol (ID), which process includes the steps of:
providing an enantiomeric mixture of an IE;
creating a reaction mixture by adding to the enantiomeric mixture a cell comprising a nucleic acid encoding, and capable of expressing, a polypeptide having enantioselective IE hydrolase activity;
incubating the reaction mixture; and
recovering from the reaction mixture: (a) an enantiopure, or a substantially enantiopure, ID; (b) an enantiopure, or a substantially enantiopure, IE; or (c) an enantiopure, or a substantially enantiopure, ID and an enantiopure, or a substantially enantiopure, IE.

3. The process of claim 1, wherein the IE is a cis-2,3-disubstituted epoxide.

4. The process of claim 1, wherein the IE is a trans-2,3-disubstituted epoxide.

5. The process of claim 1, wherein the IE is a trisubstituted epoxide.

6. The process of claim 1, wherein the IE is a methylene interrupted bis-epoxide.

7. The process of claim 2, wherein the cell is a yeast cell.

8. The process of any of claim 1, wherein the polypeptide is encoded by an endogenous gene of the cell.

9. The process of claim 2, wherein the cell is a recombinant cell and the polypeptide is encoded by a nucleic acid sequence with which the cell is transformed.

10. The process of claim 9, wherein the nucleic acid sequence is a heterologous nucleic acid sequence.

11. The process of claim 10, wherein the nucleic acid sequence is a homologous nucleic acid sequence.

12. The process of claim 1, wherein the polypeptide is a full-length yeast enantioselective internal epoxide hydrolase (YEIH).

13. The process of claim 1, wherein the polypeptide is a functional fragment of a YEIH.

14. The process of claim 1, wherein the process is carried out at a pH from 5 to 10.

15. The process of claim 1, wherein the process is carried out at a temperature of 0° C. to 70° C.

16. The process of claim 1, wherein the concentration of the IE in the reaction matrix is at least equal to the soluble concentration of the IE in water.

17. The process of claim 1, wherein the IE of the enantiomeric mixture and the obtained optically active epoxide is a compound of the general formula (A) and the ID produced by the process is a compound of the general formula (B), 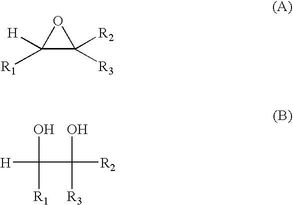
wherein,
R₁, R₂ and R₃ are, independently of each other, selected from the group consisting of a variably substituted straight-chain or branched alkyl group, a variably substituted straight-chain or branched alkenyl group, a variably substituted straight-chain or branched alkynyl group, a variably substituted cycloalkyl group as well as cycloalkenyl groups, a variably substituted aryl group, a variably substituted aryl-alkyl group, a variably substituted heterocyclic group, a variably substituted straight-chain or branched alkoxy group, a variably substituted straight-chain or branched alkenyloxy group, a variably substituted aryloxy group, a variably substituted aryl-alkyloxy group, a variably substituted alkylthio group, a variably substituted alkoxycarbonyl group, a variably substituted straight chain or branched alkylamino or alkenyl amino group, a variably substituted arylamino or arylalkylamino group, a variably substituted carbamoyl group, a variably substituted acyl group, and a functional group

18. The process of claim 17, wherein the alkyl group is a straight chain or branched alkyl group with 1 to 12 carbon atoms.

19. The process of claim 17, wherein the alkenyl group is a straight chain or branched alkenyl group having 2-12 carbon atoms.

20. The process of claim 17, wherein the alkynyl group is a straight chain or branched alkynyl group having 2-12 carbon atoms

21. The process of claim 17, wherein the cycloalkyl group is a cycloalkyl group with 3 to 10 carbon atoms.

22. The process of claim 17, wherein the cycloalkenyl group is a cycloalkenyl group with 3 to 10 carbon atoms.

23. The process of claim 17, wherein the aryl group is a phenyl, biphenyl, naphtyl, or anthracenyl group.

24. The process of claim 17, wherein the aryl-alkyl group is an aryl alkyl group with 7 to 18 carbons.

25. The process of claim 17, wherein the heterocyclic group is a 5 to 7-membered heterocyclic group containing nitrogen, oxygen or sulphur and which can be fused with a cyclic or aromatic ring having 3 to 7 carbon atoms.

26. The process of claim 17, wherein the alkoxy group is a straight chain or branched alkoxy group having 2-12 carbon atoms such as methoxy; ethoxy; propyloxy; isopropyloxy; butyloxy; isobutyloxy; tert-butyloxy; pentyloxy; hexyloxy; heptyloxy; or octyloxy.

27. The process of claim 17, wherein the alkenyloxy group is a straight chain or branched alkenyloxy group having 2-12 carbon atoms. Preferably, the alkenyloxy group is a straight chain or branched alkenyloxy groups with 2 to 8 carbon atoms.

28. The process of claim 17, wherein the aryloxy group is an aryloxy group, such as a phenoxy or naphtyloxy group. The group can optionally be substituted with an alkyl or alkenyl or alkoxy group having 1 to 4 carbon atoms or halogens.

29. The process of claim 17, wherein the arylalkyloxy group is benzyloxy or 2-phenylethyloxy.

30. The process of claim 17, wherein the alkylamino group is a straight chain or branched alkylamino group having 2-12 carbon atoms.

31. The process of claim 17, wherein the arylamino group is an arylamino group which can be substituted with an alkyl, alkenyl or alkoxy group having 1 to 4 carbon atoms, or which can be substituted with a halogen.

32. The process of claim 17, wherein the alkylamino group is benzylamino or 2-phenylethylamino.

33. The process of claim 17, wherein the alkylthio group is an alkylthio group having 1 to 8 carbon atoms.

34. The process of claim 17, wherein the alkenylthio group is a straight chain or branched alkenylthio group having 1 to 8 carbon atoms.

35. The process of claim 17, wherein the arylthio group is an arylthio group having 1 to 8 carbon atoms which can be substituted with an alkyl or alkenyl or alkoxy group having 1 to 4 carbon atoms, or which can be substituted with a halogen.

36. The process of claim 17, wherein the arylalkylthio group is an arylalkylthio group having 1 to 8 carbon atoms.

37. The process of claim 17, wherein the substituted or unsubstituted carbamoyl group is selected from the group consisting of carbamoyl, methylcarbamoyl, dimethylcarbamoyl, diethylcarbamoyl, and dipropylcarbamoyl.

38. The process of claim 17, wherein the acyl group is an acyl group with 1 to 8 carbon atoms.

39. The process of claim 17, wherein the groups are substituted, the number of substituents being one.

40. The process of claim 17, wherein the groups are substituted with more than one substituent.

41. The process of claim 17, wherein the substituents are the same.

42. The process of claim 17, wherein the substituents are different.

43. The process of claim 1, wherein the enantiomeric mixture is a racemic mixture or a mixture of any ratio of concentrations of the enantiomers.

44. The process of claim 1, which process includes adding to the reaction mixture water and at least one water-immiscible solvent.

45. The process of claim 1, which process includes adding to the reaction mixture water and at least one water-miscible organic solvent.

46. The process of claim 1, which process includes stopping the reaction when one enantiomer of the IE and/or the ID is in excess compared to the other enantiomer of the IE and/or the ID.

47. The process of claim 1, which process includes recovering continuously during the reaction an optically active IE and/or the optically active ID produced by the reaction directly from the reaction mixture.

48. The process of claim 1, wherein the yeast cell is of a yeast genus selected from the group consisting of Arxula, Brettanomyces, Bullera, Bulleromyces, Candida, Cryptococcus, Debaryomyces, Dekkera, Exophiala, Geotrichum, Hormonema, Issatchenkia, Kluyveromyces, Lipomyces, Mastigomyces, Myxozyma, Pichia, Rhodosporidium, Rhodotorula, Sporidiobolus, Sporobolomyces, Trichosporon, Wingea, and Yarrowia.

49. The process of claim 1, wherein the yeast cell is of a yeast species selected from the group consisting of Arxula adeninivorans, Arxula terrestris, Brettanomyces bruxellensis, Brettanomyces naardenensis, Brettanomyces anomalus, Brettanomyces species (e.g. Unidentified species NCYC 3151), Bullera dendrophila, Bulleromyces albus, Candida albicans, Candida fabianii, Candida glabrata, Candida haemulonii, Candida intermedia, Candida magnoliae, Candida parapsilosis, Candida rugosa, Candida tenuis, Candida tropicalis, Candida famata, Candida kruisei, Candida sp. (new) related to C. sorbophila, Cryptococcus albidus, Cryptococcus amylolentus, Cryptococcus bhutanensis, Cryptococcus curvatus, Cryptococcus gastricus, Cryptococcus humicola, Cryptococcus hungaricus, Cryptococcus laurentii, Cryptococcus luteolus, Cryptococcus macerans, Cryptococcus podzolicus, Cryptococcus terreus, Debaryomyces hansenii, Dekkera anomala, Exophiala dermatitidis, Geotrichum spp. (e.g. Unidentified species UOFS Y-0111), Hormonema spp. (e.g. Unidentified species NCYC 3171), Issatchenkia occidentalis, Kluyveromyces marxianus, Lipomyces spp. (e.g. Unidentified species UOFS Y-2159), Lipomyces tetrasporus, Mastigomyces philipporii, Myxozyma melibiosi, Pichia anomala, Pichia finlandica, Pichia guillermondii, Pichia haplophila, Rhodosporidium lusitaniae, Rhodosporidium paludigenum, Rhodosporidium sphaerocarpum, Rhodosporidium toruloides, Rhodosporidium paludigenum, Rhodotorula araucariae, Rhodotorula glutinis, Rhodotorula minuta, Rhodotorula minuta var. minuta, Rhodotorula mucilaginosa, Rhodotorula philyla, Rhodotorula rubra, Rhodotorula spp. (e.g. Unidentified species NCYC 3193, UOFS Y-2042, UOFS Y-0448, UOFS Y-0139, UOFS Y-0560), Rhodotorula aurantiaca, Rhodotorula spp. (e.g. Unidentified species NCYC 3224), Rhodotorula sp. “mucilaginosa”, Sporidiobolus salmonicolor, Sporobolomyces holsaticus, Sporobolomyces roseus, Sporobolomyces tsugae, Trichosporon beigelii, Trichosporon cutaneum var. cutaneum, Trichosporon delbrueckii, Trichosporon jirovecii, Trichosporon mucoides, Trichosporon ovoides, Trichosporon pullulans, Trichosporon spp. (e.g. Unidentified species NCYC 3210, NCYC 3212, NCYC 3211, UOFS Y-0861, UOFS Y-1615, UOFS Y-0451, UOFS Y-0449, UOFS Y-2113), Trichosporon moniliiforme, Trichosporon montevideense, Wingea robertsiae, and Yarrowia lipolytica.

50. A method for producing a polypeptide, which process includes the steps of:
providing a cell comprising a nucleic acid encoding and capable of expressing a polypeptide that has enantioselective IE hydrolase activity; and
culturing the cell.

51. The method of claim 50, further comprising recovering the polypeptide from the culture.

52. The method of claim 50, wherein the cell is a yeast cell.

53. The method of claim 50, wherein the polypeptide is a full-length YEIH.

54. The method of claim 50, wherein the polypeptide is a functional fragment of a YEIH.

55. The method of claim 50, wherein the polypeptide is encoded by an endogenous gene of the cell.

56. The method of claim 50, wherein the cell is a recombinant cell and the polypeptide is encoded by a nucleic acid sequence with which the cell is transformed.

57. The method of claim 56, wherein the nucleic acid sequence is a heterologous nucleic acid sequence.

58. The method of claim 56, wherein the nucleic acid sequence is a homologous nucleic acid sequence.

59. A crude or pure enzyme preparation which comprises an YEIH.

60. A substantially pure culture of cells, a substantial number of which comprise a nucleic acid encoding, and are capable of expressing, a polypeptide having enantioselective IE hydrolase activity.

61. An isolated cell, the cell comprising a nucleic acid encoding a polypeptide having enantioselective EE hydrolase activity, the cell being capable of expressing the polypeptide.

62. An isolated DNA comprising:
(a) a nucleic acid sequence that encodes a polypeptide that has enantioselective IE hydrolase activity and that hybridizes under highly stringent conditions to the complement of a sequence selected from the group consisting of SEQ. ID. NOs: 8, 9, 10, 11, 12, 13, and 14, or
(b) the complement of the nucleic acid sequence.

63. The DNA of claim 62, wherein the nucleic acid sequence encodes a polypeptide comprising an amino acid sequence selected from the group consisting of SEQ. ID. NOs: 1, 2, 3, 4, 5, 6, and 7.

64. The DNA of claim 62, wherein the nucleic acid sequence is selected from the group consisting of SEQ ID NOs: 8, 9, 10, 11, 12, 13, and 14.

65. An isolated DNA comprising:
(a) a nucleic acid sequence that is at least 55% identical to a sequence selected from the group consisting of SEQ ID NOs: 8, 9, 10, 11, 12, 13, and 14; or
(b) the complement of the nucleic acid sequence,
wherein the nucleic acid sequence encodes a polypeptide that has enantioselective IE hydrolase activity.

66. An isolated DNA comprising:
(a) a nucleic acid sequence that encodes a polypeptide consisting of an amino acid sequence that is at least 55% identical to a sequence selected from the group consisting of SEQ ID NOs: 1, 2, 3, 4, 5, 6, and 7 or
(b) the complement of the nucleic acid sequence,
wherein the polypeptide has enantioselective IE hydrolase activity.

67. An isolated polypeptide encoded by the DNA of claim 62.

68. An isolated polypeptide comprising an amino acid sequence that is at least 55% identical to SEQ. ID. NOs: 1, 2, 3, 4, 5, 6, or 7, the polypeptide having enantioselective IE hydrolase activity.

69. The polypeptide of claim 67, comprising:
(a) an amino acid sequence selected from the group consisting of SEQ. ID. NOs; 1, 2, 3, 4, 5, 6 and 7 or a functional fragment of the sequence; or
(b) the sequence of (a), but with no more than five conservative substitutions,
wherein the polypeptide has enantioselective internal epoxide hydrolase activity.

70. An isolated antibody that binds to the polypeptide of claim 67.

71. The antibody of claim 70, wherein the antibody is a polyclonal antibody.

72. The antibody of claim 70, wherein the antibody is a monoclonal antibody.
